(19) United States
(12) Patent Application Publication
(10) Pub. No.: US 2025/0376699 A1
(43) Pub. Date: Dec. 11, 2025

(54) IMPACT OF ADIPOCYTE-RELEASED ADIPOMES IN CHAGAS CARDIOMYOPATHY ON CARDIAC METABOLIC AND IMMUNE REGULATION AND IN A BREAST CANCER MODEL

(71) Applicant: HACKENSACK MERIDIAN HEALTH, INC., Edison, NJ (US)

(72) Inventors: Jyothi F. Nagajyothi, Wayne, NJ (US); Hariprasad Thangavel, Newark, NJ (US)

(73) Assignee: HACKENSACK MERIDIAN HEALTH, INC., Edison, NJ (US)

(21) Appl. No.: 19/233,485

(22) Filed: Jun. 10, 2025

Related U.S. Application Data
(60) Provisional application No. 63/658,664, filed on Jun. 11, 2024.

(51) Int. Cl. C12N 15/88; C12Q 1/6883; G01N 33/92

(52) U.S. Cl. CPC C12N 15/88; C12Q 1/6883; G01N 33/92; C12Q 2600/158

(57) ABSTRACT

In the body, adipose tissue is comprised of adipocytes as well as various other cell types, including immune cells, that may contribute to the overall extracellular vesicle pool. Adiponectin, a protein hormone produced by fat cells, and fatty acid binding protein 4 (FABP4) were selected as markers to isolate adipocyte-specific EVs/adipomes from the total pool of white adipose tissue-derived EVs based on in vitro data showing that they colocalized with Annexin V, an apoptosis marker, on the surface of budding apoptotic bodies. Intact L-adipomes (large-adipomes) and S-adipomes (small-adipomes) were successfully and selectively enriched from large-EVs and small-EVs, respectively. Immunoblotting analysis confirmed the presence of adiponectin, FABP4, Annexin V, and perilipin in both L- and S-adipomes, providing further evidence of their adipocyte origin.

### CROSS-REFERENCE TO RELATED APPLICATIONS

This application claims the benefit of priority to U.S. Provisional Application 63/658,664, filed Jun. 11, 2024, and entitled ADIPOCYYTE-RELEASED ADIPOMES IN CHAGAS CARDIOMYOPATHY. IMPACT ON CARDIAC METABOLIC AND IMMUNE REGULATIONS, the contents of which are incorporated by reference herein in their entirety.

### BACKGROUND OF THE INVENTION

Chagas disease (CD), caused by Trypanosoma cruzi, affects 8 million people in Latin America and 400,000 in the rest of the world, the latter mainly due to migration and blood transfusion.^{1,2} Approximately 30% of infected individuals develop chronic symptomatic disease after several years or decades of initial infection, including dilated chronic cardiomyopathy (CCM), which can be fatal.³ Furthermore, recent reports suggest that individuals in the acute phase can also progress to a chronic form of acute cardiomyopathy (ACM).4 Acute infection may lead to generalized cardiac enlargement, affecting all four cardiac chambers, and may be accompanied by pericardial effusions.⁵ While myocarditis arising during acute infection generally resolves with increased anti-inflammatory signaling in the heart, which is associated with cardiac re-modeling, deregulated cardiac remodeling post-infection may contribute to the pathogenesis of Chagas cardiomyopathy, characterized by hypertrophied cardiomyopathy, dilated cardiomyopathy, and ultimately, heart failure. Although existing anti-parasitic drugs demonstrate significant efficacy in the early, acute CD stages, treatments available to prevent CCM are ineffective and have more severe side effects.⁶

The development of CCM is influenced by factors such as host metabolic and immunological status.⁷ While pro-inflammatory signaling during acute infection contributes to myocarditis, CCM progression depends on cardiac energy metabolism, immune signaling, and the parasite's presence, regulating cardiac remodeling during the post-acute asymptomatic phase. Our studies of murine CD models have also revealed increased adipogenesis and altered lipid metabolism, as well as mitochondrial dysfunction, oxidative stress, and endoplasmic reticulum (ER) stress in the myocardium.⁸⁻¹¹ It has been shown that cardiac metabolic status influences the functions of heart immune cells and vice versa.¹² Thus, understanding the mechanism(s) promoting adipogenic/lipogenic signaling and mitochondrial dysfunction in the heart during cardiac remodeling is essential for the development of effective therapeutic interventions for CCM.

Adipose tissue (AT), which is mainly comprised of adipocytes, plays an important role in regulating whole-body immunity and metabolic homeostasis.¹³⁻¹⁵ Cardiac AT is a dynamic organ with metabolic activity, actively participating in the maintenance of lipid and energy balance in the heart. Beyond serving as an energy source for the myocardium, it functions as a buffer protecting the heart from lipotoxicity, particularly in the presence of elevated circulating free fatty acids (FFAs).¹⁶ We have shown that T. cruzi invades adipocytes, whereupon AT serves as a parasite reservoir in both patients with CD and murine CD models.^{17,18} T. cruzi relies on adipocytes for cholesterol, as it cannot synthesize it independently.¹⁹ Adipocytes provide nutrients for persistent T. cruzi and facilitate the parasite evasion of host immunity.²⁰ Previously, we showed a strong correlation between loss of body fat and increased ventricular dilation in T. cruzi-infected mice.¹⁷ Our studies also demonstrated that T. cruzi persistence in AT disrupts host lipid metabolism, increasing CCM risk by causing adipocyte cell death.¹¹ Our prior studies of murine CD models have revealed a connection between the loss of body fat resulting from induced adipocyte apoptosis and an escalation of cardiomyopathy.¹¹ This association was observed during both the acute and indeterminate stages of T. cruzi infection, indicating a crucial role for pathological adipocytes in the regulation of cardiomyopathy in CD.¹¹ In this study, our primary objective was to elucidate the immunometabolic consequences of adipocyte apoptosis and its impact on cardiac remodeling, which in turn influences the pathogenesis of cardiomyopathy in CD.

Building upon our previous findings, we developed a hypothesis that T. cruzi infection induces the apoptosis of adipocytes, leading to the release of extracellular vesicles, a.k.a. “adipomes”, and that these adipomes may regulate the immune and metabolic functions of the myocardium, heightening the risk of cardiomyopathy during T. cruzi infection. The current study utilized both in vitro and in vivo models to test this hypothesis by investigating how pathological adipocyte-derived adipomes regulate immune cell activation and cardiomyocyte dysfunction in post-acute T. cruzi infection state. To isolate intact adipomes from the plasma and AT of infected mice, we developed an innovative method where the adipomes could be selectively purified based on their size (large (L) or small (S)) and unique surface markers. This purification method overcomes the limitations in isolating adipocyte-specific adipomes from AT in its microenvironment.^{21,22} We characterized the adipomes using transmission electron microscopy (TEM) and analyzed their lipid contents, showing that they contained active lipid biomolecules whose patterns varied between adipomes derived from healthy versus pathological adipocytes. Additionally, we assessed the functional significance of L-adipomes isolated from the plasma of T. cruzi-infected mice by injecting them into the hearts of wild-type and post-acute Cha-gas mice. We observed that adipome treatment increased inflammatory and adipogenic/lipogenic signaling and markers of ER stress, as well as elevated levels of atrial natriuretic peptide (ANP) and b1-adrenergic receptor (b1-AR) in the mouse hearts. Finally, we found that the ultra-sound-guided intramyocardial injection of plasma-derived infection-associated L-adipomes (P-ILA) significantly altered cardiac morphology, increasing the risk of cardiomyopathy in wild-type mice compared to treatment with the vehicle alone. Together, these findings offer new mechanistic insights into the immune and metabolic alterations triggered by the T. cruzi infection of AT that regulate cardiac remodeling and promote cardiac pathogenesis during CD.

### SUMMARY OF THE INVENTION

According to one aspect, the present disclosure provides a method for early diagnosis of a subject at risk for a pathology comprising alterations in metabolic and inflammatory signaling in phagocytic and nonphagocytic target cells, the method comprising: selectively purifying from a population of various cell types that include adipocytes, a subpopulation of apototic adipocytes or dying adipocytes expressing adipocyte markers including adiponectin, FABP4 or both; isolating from the subpopulation of adipocytes including apoptotic adipocytes a population of adipocyte-specific extracellular vesicles (adipomes) derived from the population of total WAT-derived extracellular vesicles; enriching intact large adipomes (L-adipomes) from large EVs and small adipomes (S-adipomes) from small EVs that express the adipocyte markers adiponectin and FABP4 by positive selection employing adiponectin and FABP4 antibodies; extracting cargo of the L-adipomes and the S-adipomes and determining their lipid profile; comparing mRNA expression levels of the L-adipomes and S-adipomes from the subject to mRNA expression levels of a healthy control including adiponectin receptors AdipoR1 and AdipoR2; and determining a level of expression of one or more genes encoding a panel of proteins that function in lipolytic signaling, lipogenesis, mitochondrial signaling, inflammation or a combination thereof in target cells; wherein early diagnosis of the pathology can lead to improved outcome for the subject.

According to some embodiments of the method, a source of the adipomes population is plasma or white adipose tissue (WAT) comprising an adipocyte population including the subpopulation of apoptotic adipocytes or dying adipocytes and a non-adipocyte population comprising adipocyte precursor cells.

According to some embodiments, the L-adipomes are derived from large EVs comprising apoptotic bodies and macrovesicles and the S-adipomes are derived from small EVs comprising microvesicles and exosomes.

According to some embodiments, the pathology affects lipid metabolism, mitochondrial oxidative phosphorylation, inflammation, or a combination thereof.

According to some embodiments, the L-adipomes and S-adipomes derived from the adipocytes express adiponectin, FABP4, annexin V and perilipin.

According to some embodiments, size of the L-adipomes ranges from 200-400 nm and size of the S-adipomes ranges from 30-80 nm.

According to some embodiments, the panel of genes includes: a gene encoding a protein of lipolytic signaling comprising beta-2-adrenergic receptor (ADRB2) protein; a gene encoding a protein of lipogenesis comprising one or more of lipogenic SREBP1a and SREBP1c proteins; genes encoding a protein of mitochondrial signaling comprising one or more of NADHD, ND1, ND2, SDHC, CYTB, COX1A, COX5A, APT6, ANT1 or PGC-1a protein; and a gene encoding a protein of inflammatory signaling comprising TNFa, IFNg, or both proteins.

According to some embodiments, the pathology comprises polarization of a macrophage population derived from the subject at risk compared to a healthy subject.

According to some embodiments, the pathology places at risk cardiomyocytes, cardiac fibroblasts or both. According to some embodiments, the pathology in an untreated subject progresses to hypertrophied cardiomyopathy, dilated cardiomyopathy and heart failure.

According to some embodiments, the hypertrophied cardiomyopathy comprises cardiac remodeling and cardiomyocyte dysfunction.

According to some embodiments, a source of the pathology is an infection. According to some embodiments, the infection is a parasitic infection. According to some embodiments, the parasitic infection is an infection with Trypanosoma cruzi, a causative agent of Chagas disease.

According to another aspect, the present disclosure provides a method for modulating adipogenic signaling in a target cell population comprising: selectively purifying from a population of adipocytes a subpopulation of apototic adipocytes or dying adipocytes expressing adipocyte markers including adiponectin, FABP4 or both; isolating from the subpopulation of apoptotic and dying adipocytes a population of adipocyte-specific extracellular vesicles (adipomes) derived from the population of total WAT-derived extracellular vesicles, wherein size of the adipomes ranges from 200 nm to 1100 nm; enriching intact large adipomes (L-adipomes) from large EVs and small adipomes (S-adipomes) from small EVs that express the adipocyte markers adiponectin and FABP4 by positive selection employing adiponectin and FABP4 antibodies; extracting cargo of the L-adipomes and the S-adipomes and determining their cargo profile, wherein the cargo of the adipomes includes adipogenic mRNA; treating the population of target cells with the adipomes comprising the adipogenic mRNA cargo; and modulating gene expression of adiponectin and downstream genes regulated by adiponectin.

According to some embodiments of the method, a source of the adipomes population is plasma or white adipose tissue (WAT) comprising an adipocyte population including a subpopulation of apoptotic adipocytes or dying adipocytes and a non-adipocyte population comprising adipocyte precursor cells.

According to some embodiments, the L-adipomes are derived from large EVs comprising apoptotic bodies and macrovesicles and the S-adipomes are derived from small EVs comprising microvesicles and exosomes.

According to some embodiments, the target cell population comprises macrophages, fibroblasts or both.

According to some embodiments, the adipogenic genes comprise mRNA for Adipoq, Fabp4, and Pparg.

According to some embodiments, the downstream genes regulated by adiponectin include Ppara and adiponectin receptor R2 (AdipoR2).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A, FIG. 1B, FIG. 1C, FIG. 1D, FIG. 1E, FIG. 1F, and FIG. 1G show that T. cruzi infection induces adipocyte apoptosis, releasing adipomes that regulate adipogenic signaling. FIG. 1A shows an immunoblot analysis of cleaved Caspase 7 expression in the lysates of 3T3-L1 adipocytes (uninfected, TNFa-treated, and T. cruzi infected) (n=3/group). Bar graph values were derived from densitometry analysis and by normalizing target protein expression to b-Actin. FIG. 1B shows fold change expression of CASP3, RIPK3, and MLKL mRNA transcripts (normalized to HPRT) in cultured human adipocytes (uninfected, TNFa-treated and T. cruzi infected) (n=3). FIG. 1C and FIG. 1D show adipome size distribution and absolute concentration as determined by Spectradyne nCS1 with a C-900 microfluidic cartridge. Both mouse and human adipomes were derived from cultured 3T3-L1 and human adipocyte-conditioned media, respectively. Inset: A (blue)=uninfected, B (green)=TNFa-treated, C (red)=T. cruzi infected. FIG. 1E shows a PCR analysis of adipogenic (Adipoq, Fabp4, and Pparg), apoptotic (Chop) and extracellular vesicle (Tsg101 and Cd13) marker genes in 3T3-L1-derived adipomes. Lanes 1, 2, and 3 represent 3T3-L1-derived adipomes from three independent cultures. Product size: Adipoq, 192 bp; Fabp4, 133 bp; Pparg, 132 bp; Chop, 118 bp; Tsg101, 103 bp; and Cd13, 121 bp. FIG. 1F and FIG. 1G show fold change expression of Adipoq mRNA transcripts (normalized to Hprt) in RAW macrophages (FIG. 1F) and human fibroblasts (FIG. 1G) treated with 3T3-L1- and human adipocyte-derived adipomes, respectively, at 1:1 and 1:50 cell-to-adipome ratio for 48 h (n=3). All treatment groups (Treated-A, -B and -C) were compared to untreated groups. The #symbol indicates comparison between Treated-B and -C. Treated-A=adipomes from uninfected adipocytes; Treated-B=adipomes from TNFa-treated adipocytes; and Treated-C=adipomes from T. cruzi-infected adipocytes. Data are represented as mean G SEM. (*/#p<0.05, **p % 0.01, ***/###p % 0.001 and ****p % 0.0001).

FIG. 2A, FIG. 2B, FIG. 2C, FIG. 2D, FIG. 2E, FIG. 2F and FIG. 2G show that adiponectin enables selective capture of adipomes from murine white adipose tissue (WAT). FIG. 2A shows an immunoblot analysis of phospho-Perilipin, cleaved Caspase 7 and Annexin V expression in the WAT lysates of infected (20 DPI) and uninfected C57BL/6J mice (n=4-8 per group). Bar graph values were derived from densitometry analysis and by normalizing target protein expression to GDI. Error bars indicate the standard error of the mean (*p<0.05). FIG. 2B shows an immunoblot analysis of adiponectin in WAT-derived large-EVs (L-EV) and small-EVs (S-EV). Black arrow points to the 25 kDa marker band on the pre-stained protein ladder. Adiponectin band appears at 26/30 kDa. Lanes: C, control WAT lysate; L Ev, large-EV; and S Ev, small-EV. FIG. 2C shows surface immunofluorescence staining of 3T3-L1 adipocytes show the co-localization of Adiponectin (green) with Annexin V (red) (top panel) and FABP4 (red) with Annexin V (green) (bottom panel) on budding apoptotic bodies. Scale bar, 50 mm. FIG. 2D shows an immunoblot analysis of adiponectin in WAT-derived large-adipomes and small-adipomes. The black arrow points to the 25 kDa marker band on pre-stained protein ladder. Adiponectin band appears at 26/30 kDa. Lanes: C, control WAT lysate; L, large-adipomes; and S, small-adipomes. FIG. 2E shows an immunofluorescence assay (IFA) of bead-bound adipomes stained with Adiponectin-AF488 (top panel) and FABP4-AF488 (bottom panel) imaged on the FITC channel along with their corresponding bright field (BF) images. Scale bar, 5 mm. FIG. 2F shows WAT-derived adipome size distribution and absolute concentration as determined by Spectradyne nCS1 with a C-2000 microfluidic cartridge. Inset: L (blue), large-adipomes; S (green), small-adipomes. Gate: G1, particle concentration at size range of 250-400 nm diameter showing more small-adipomes than large-adipomes; and G2, particle concentration at size range of 700-1100 nm diameter showing mostly large-adipome distribution. FIG. 2G shows transmission Electron Microscopy (TEM) images of small-adipomes (left, scale bar 80 nm) and large-adipomes (right, scale bar 100 nm) with a direct magnification of 20,000×. Black arrow indicates the budding scars on L-adipomes.

FIG. 3A, FIG. 3B, FIG. 3C, FIG. 3D, FIG. 3E, FIG. 3F, FIG. 3G, FIG. 3H and FIG. 3I show that the lipidome of L-adipomes is distinct from that of S-adipomes. FIG. 3A and FIG. 3G shows a principal component analysis (PCA) score plot of the lipid profiles from CLA and ILA (FIG. 3A), and ILA and ISA (FIG. 3G) showing clusters of two groups. FIG. 3B and FIG. 3H show a heatmap representation of the clustering analysis of top 25 differentially abundant lipid molecular species (with lowest FDR-adjusted p-values) based on z-scores for the normalized, transformed, and scaled data between CLA and ILA (FIG. 3B), and ILA and ISA (FIG. 3H). For class names, red represents CLA or ILA and green represents ILA or ISA. For the abundance of each lipid, red represents high, and blue represents low. FIG. 3C and FIG. 3I show volcano plots, which represent significantly altered lipid metabolites in ILA compared to CLA (FIG. 3C), and ISA compared to ILA (FIG. 3I). Red dots indicate upregulated lipids and blue dots indicate downregulated lipids. FIG. 3D shows KEGG pathway analysis, which revealed seven dysregulated metabolic pathways enriched in ILA compared to CLA. All matched pathways were plotted according to p-value and pathway impact score from pathway enrichment analysis and pathway topology analysis, respectively. Color gradient: yellow, higher p-value; and red, lower p-value. Circle size: large, higher impact score; and small, lower impact score. FIG. 3E and FIG. 3F are pathway representations of Sphingolipid metabolism (FIG. 3E) and Glycerophospholipid metabolism (FIG. 3F) with lipid metabolites (highlighted in red) enriched in ILA. Data represents the mean (n=3 per group). Abbreviations: CLA, control large-adipomes; ILA, infected large-adipomes; and ISA, infected small-adipomes.

FIG. 4A, FIG. 4B, FIG. 4C, FIG. 4D, FIG. 4E, and FIG. 4F show that infected L-adipomes alter immunometabolic signaling and macrophage polarization (FIG. 4A, FIG. 4B, FIG. 4C, FIG. 4D) Changes in the expression of genes involved in adipogenesis (FIG. 4A), lipid metabolism (FIG. 4B), and immune and inflammatory signaling (FIG. 4C and FIG. 4D) based on mRNA fold change values (normalized to Hprt) in macrophages treated with CLA and ILA for 48 h as demonstrated by custom RT2 Profiler qPCR array analysis. Fold change was calculated using Qiagen data analysis spreadsheet. (FIG. 4E and FIG. 4F) Fold change expression of Arg1 mRNA transcript (FIG. 4E) and mRNA levels of genes involved in mitochondrial oxidative phosphorylation (FIG. 4F) (normalized to Gapdh) in macrophages treated with CLA and ILA for 48 h as demonstrated by qPCR analysis (n=3). Data are represented as mean G SEM. (*p<0.05, **p % 0.01, and ***p % 0.001).

FIG. 5A, FIG. 5B, and FIG. 5C show that adipomes play a major regulatory role in cardiomyocytes functioning. FIG. 5A, FIG. 5B, and FIG. 5C show fold change in mRNA transcript levels of genes involved in adipogenic and lipogenic signaling (FIG. 5A), mitochondrial oxidative phosphorylation (FIG. 5B), and inflammatory signaling (FIG. 5C) (normalized to Gapdh) in murine primary cardiomyocytes treated with CLA, ILA, CSA, or ISA for 48 h and compared to untreated cardiomyocytes as demonstrated by qPCR analysis (n=3). All adipome-treated groups (CLA, ILA, CSA, and ISA) were compared to untreated groups. Data are represented as mean G SEM. (*p<0.05, **p % 0.01, and ***p % 0.001).

FIG. 6 shows that circulatory adipomes induce ER stress in cardiomyocytes. The left panel shows an immunoblot analysis of b1-AR and CHOP expression in murine primary cardiomyocytes treated with plasma-derived adipomes (P-CLA, P-ILA, P-CSA, and P-ISA) and naive cells (n=3/group). Bar graph values in the middle panel (b1-AR) and the right panel (CHOP) were derived from densitometry analysis of the immunoblot and by normalizing target protein expression to GDI. The “&” symbol denotes significance calculated between different adipome-treated groups. Data are represented as mean G SEM. (*p<0.05, **p % 0.01, and ***p %0.001).

FIG. 7A, FIG. 7B, FIG. 7C, FIG. 7D, FIG. 7E, and FIG. 7F show that P-ILA cause mitochondrial dysfunction in primary cardiomyocytes. FIG. 7A shows a Seahorse Mito Stress assay showing the oxygen consumption rate (OCR) from plasma-adipome treated (P-CLA and P-ILA) and untreated mouse cardiomyocytes (n R 6). Data normalized to nuclear content by staining and fluorescence cell counting. FIG. 7B, FIG. 7C, FIG. 7D, FIG. 7E, and FIG. 7F are representative graphs depicting the different mitochondrial respiration parameters evaluated, such as basal respiration (FIG. 7B), maximal respiration (FIG. 7C), proton leak (FIG. 7D), spare respiratory capacity (FIG. 7E), and ATP production (FIG. 7F). Data are represented as mean G SEM. (*p<0.05, **p % 0.01, ***p % 0.001, and ***p % 0.0001).

FIG. 88A, FIG. 8B, FIG. 8C, FIG. 8D, and FIG. 8E show that P-ILA elevate the risk for cardiomyopathy in murine CD model during post-acute phase. FIG. 8A, FIG. 8B, FIG. 8C, FIG. 8D, and FIG. 8E show immunoblot analysis of inflammation (FIG. 8A: TNFa, IFNg, IL6), immune cell infiltration (FIG. 8A: F4/80), lipogenesis (FIG. 8B: SREBP1), lipid droplet formation (FIG. 8B: Perilipin-1), lipid hydrolysis (FIG. 8B: phospho-Perilipin), lipid oxidation (FIG. 8B: PPARa), mitochondrial oxidative phosphorylation (FIG. 8C: SDHA, Cytochrome c, COX IV, HSP60), ER stress (FIG. 8D: BiP, phospho-eIF2a, PDI), apoptosis (FIG. 8D: CHOP, FIG. 8E: Caspase 7, Cleaved-Caspase 7), and necrosis (FIG. 8E: BNIP3) markers in heart lysates of uninfected and post-acute T. cruzi—infected mice treated with intracardiac injections of either P-ILA or vehicle. Bar graph values were derived from densitometry analysis and by normalizing target protein expression to GDI. Lanes: 1, uninfected vehicle-injected; 2, uninfected P-ILA-injected; 3, T. cruzi-infected vehicle-injected; and 4, T. cruzi-infected P-ILA-injected murine heart tissue lysates. “&” symbol indicates significance calculated between uninfected and T. cruzi-infected P-ILA injections. Data are represented as mean G SEM. (*p<0.05, **p % 0.01, and ***p % 0.001).

FIG. 9A and FIG. 9B show that intracardiac P-ILA treatment increases cardiac adiponectin (APN) and Atrial natriuretic peptide (ANP) in murine CD model. FIG. 9A shows immunoblot analysis of adiponectin and b1-AR expression in heart lysates of uninfected and post-acute T. cruzi-infected mice treated with intracardiac injections of either P-ILA or vehicle. Bar graph values were derived from densitometry analysis and by normalizing target protein expression to GDI. Lanes: 1, uninfected vehicle-injected; 2, uninfected P-ILA-injected; 3, T. cruzi-infected vehicle-injected; and 4, T. cruzi-infected P-ILA-injected murine heart tissue lysate. The “&” symbol indicates significance calculated between uninfected and T. cruzi-infected P-ILA injections. FIG. 9B shows representative immunohistochemistry (IHC) images of vehicle- and P-ILA-injected heart tissue from uninfected and T. cruzi-infected mice showing ANP immunostaining visualized with DAB and counterstained with hematoxylin. Dot plot shows staining intensity in O.D. (mean gray value) obtained by color deconvolution analysis. Data are represented as mean G SEM. (**p % 0.01, and ***p % 0.001).

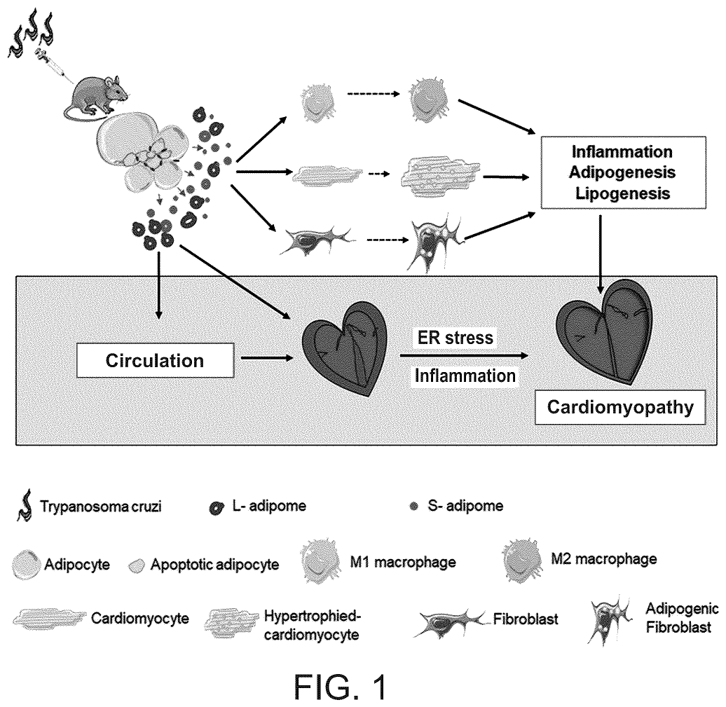

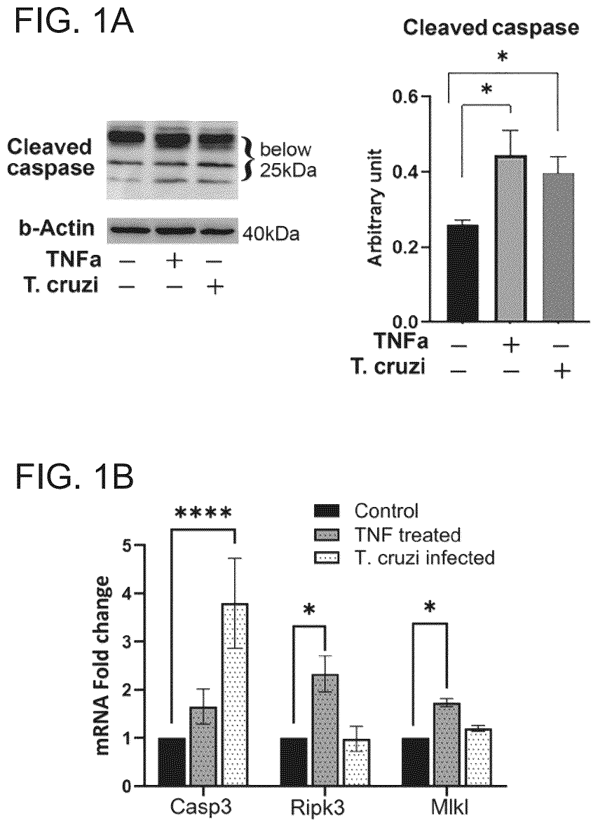

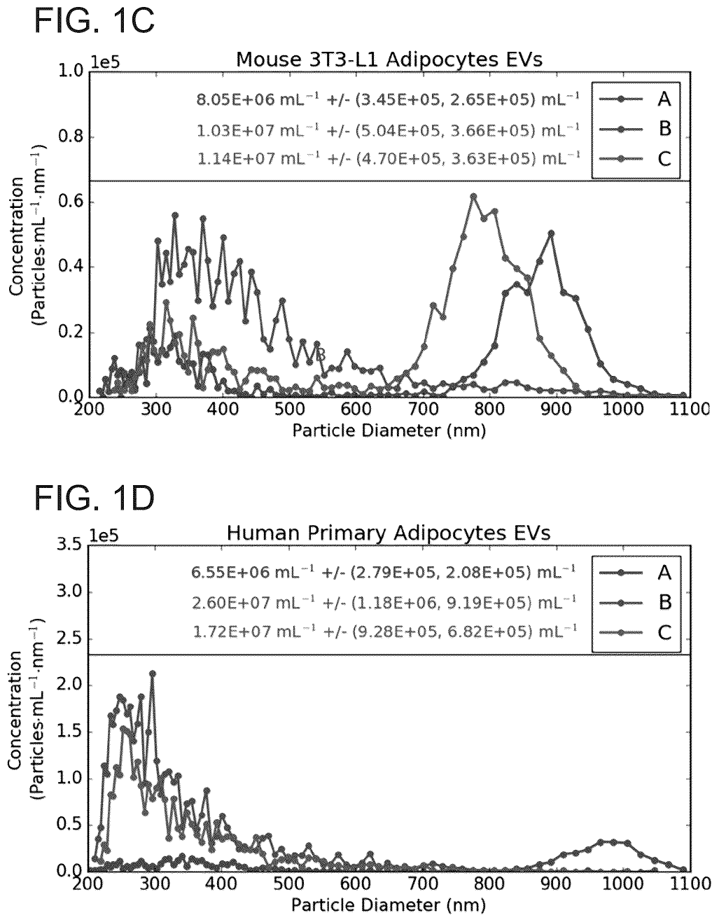

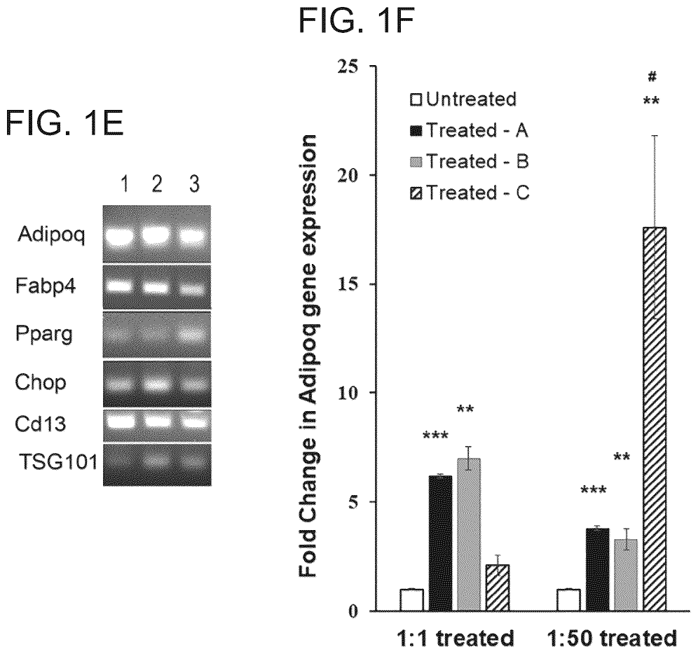

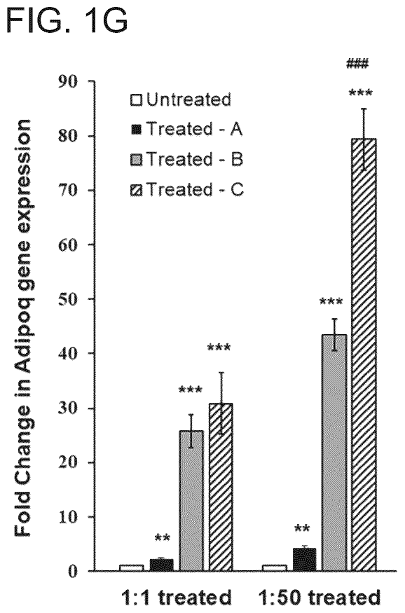

FIG. 10A, FIG. 10B and FIG. 10C show that T. cruzi infection induces adipocyte apoptosis, releasing adipomes that regulate adipogenic signaling. The figure is related to STAR Methods and FIG. 1. FIG. 10A is a schematic representation of adipome isolation from cultured adipocytes, plasma, and murine white adipose tissue. FIG. 10B and FIG. 10C show fold change expression of Ppara, Pparg, Tnfa, Adipor1 and Adipor2 mRNA transcripts (normalized to Hprt) in RAW macrophages (FIG. 10B) and human fibroblast cells (FIG. 10C) treated with 3T3-L1 and human adipocyte derived adipomes, respectively, at 1:1 and 1:50 cell to adipome ratio for 48 hours (n=3). All treated groups (treated-A, treated-B and treated-C) were compared to an untreated control group. The #symbol indicates comparison between Treated-B and Treated C. Treated-A, adipomes from uninfected adipocytes; Treated-B, adipomes from TNFa-treated adipocytes; and Treated-C, adipomes from T. cruzi infected adipocytes. Data are represented as mean±SEM (* p<0.05; **p≤0.01; *** p≤0.001).

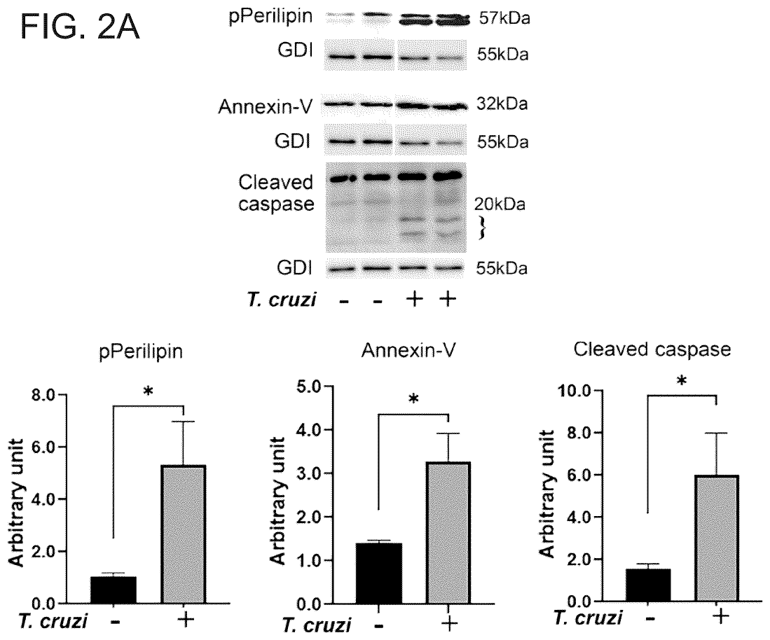

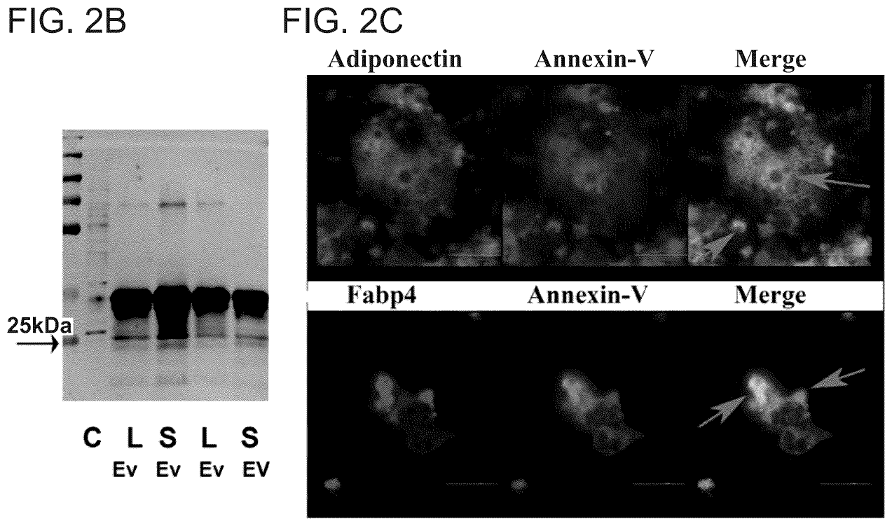

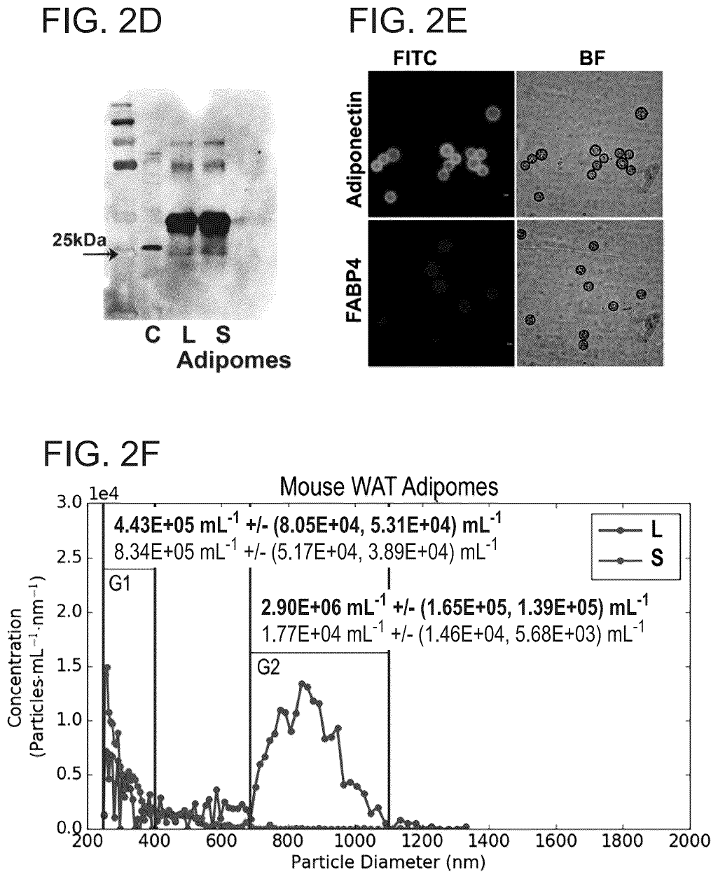

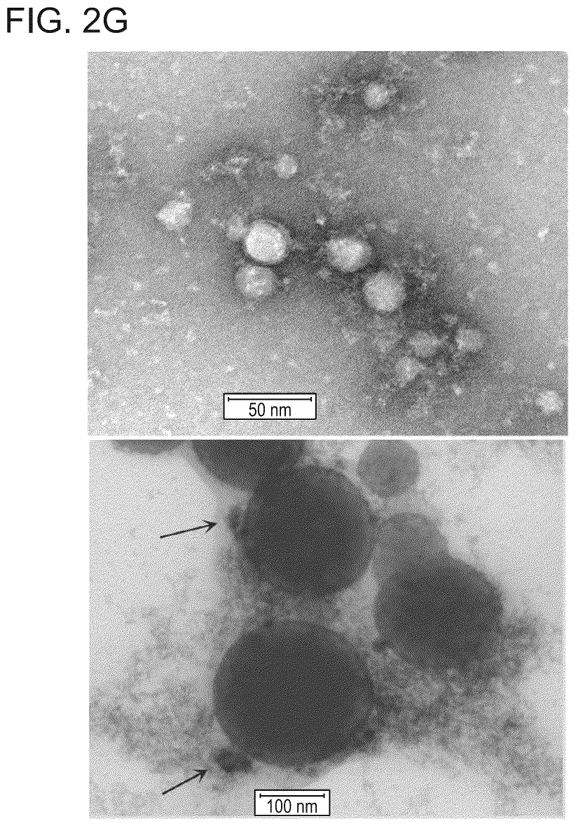

FIG. 11A, FIG. 11B, FIG. 11C, FIG. 11D, FIG. 11E, FIG. 11F, and FIG. 11G show that adiponectin enables selective capture of adipomes from murine white adipose tissue (WAT); the figure is related to FIG. 2. FIG. 11A shows Magnetic Resonance Imaging (MRI) of control (left) and acute T. cruzi infected (right) mouse. White arrow indicates loss of pericardial fat in infected (right) compared to control (left) mouse. FIG. 11B shows adiponectin and FABP4 antibody enriched mouse 3T3-L1 adipome size distribution and absolute concentration as determined by Spectradyne nCS1 with a C-900 microfluidic cartridge. Inset: A (black) untreated; and B(gray) TNFa-treated samples. Gate: particle concentration at size range of 250-500 nm diameter showing ˜4-fold more adipome abundance in TNF-treated sample than untreated. FIG. 11C shows validation of adipome cargo by immunoblotting analysis. Presence of Adiponectin (26/30 kDa), FAB4 (14 kDa), Annexin V (32 kDa), and Perilipin (62 kDa) in adipomes were shown. Black arrow indicates target protein band. Lanes: C, control WAT lysate; L, large-adipomes; and S, small-adipomes. FIG. 11D shows surface IFA of untouched magnetic streptavidin beads stained with Goat anti-mouse and Goat anti-rat IgG Alexa Fluor 488 cocktail imaged on FITC channel along with corresponding bright field (BF) image. Scale bar, 10 m. FIG. 11E, FIG. 11E, FIG. 11G show TEM image of small-adipomes (FIG. 11E), magnetic streptavidin bead-bound large-adoipomes (FIG. 11F), and eluted large-adipomes (FIG. 11G). Scale bar, 80 nm (FIG. 11E) and 100 nm (FIG. 11F, FIG. 11G). Black arrows (FIG. 11F) indicate large-adipomes bound on the magnetic bead surface.

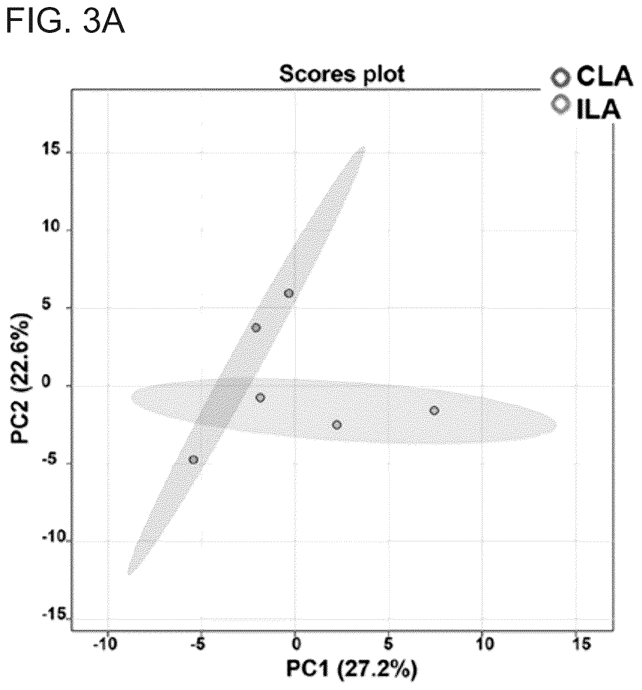

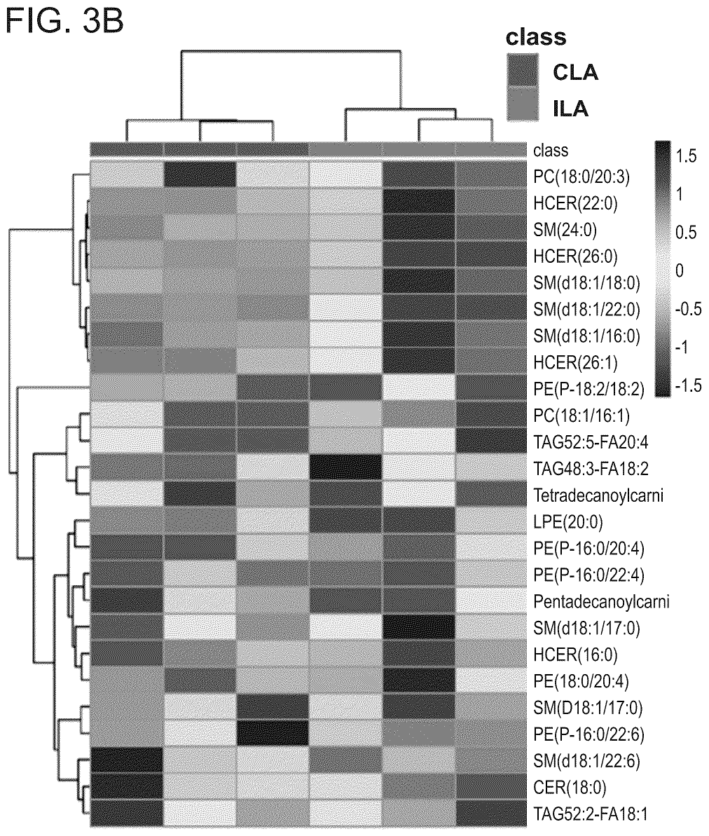

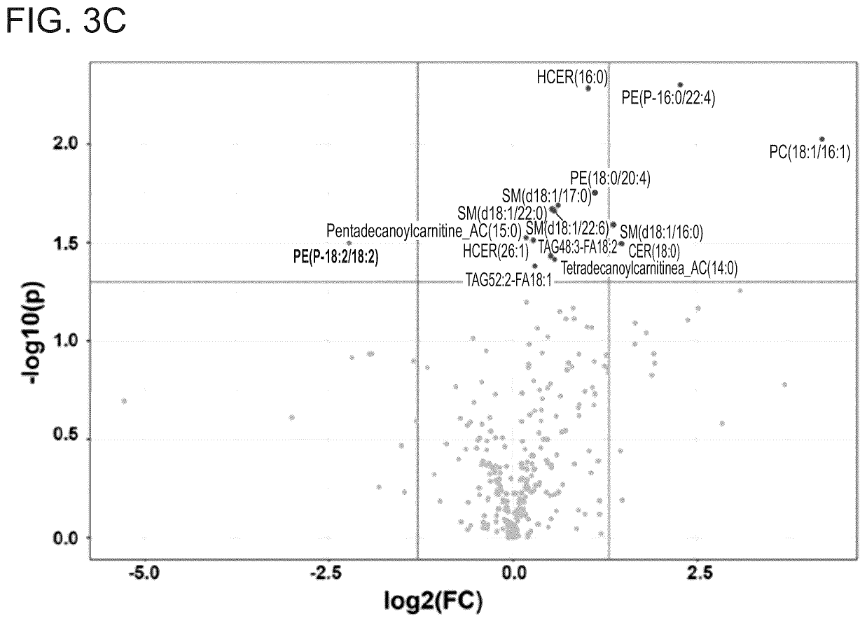

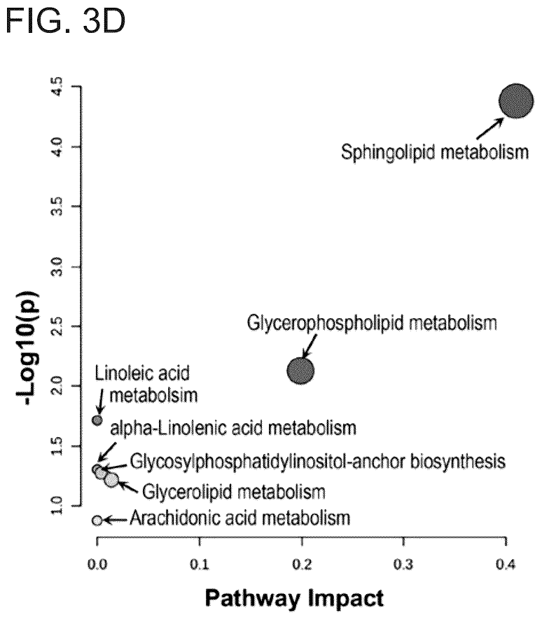

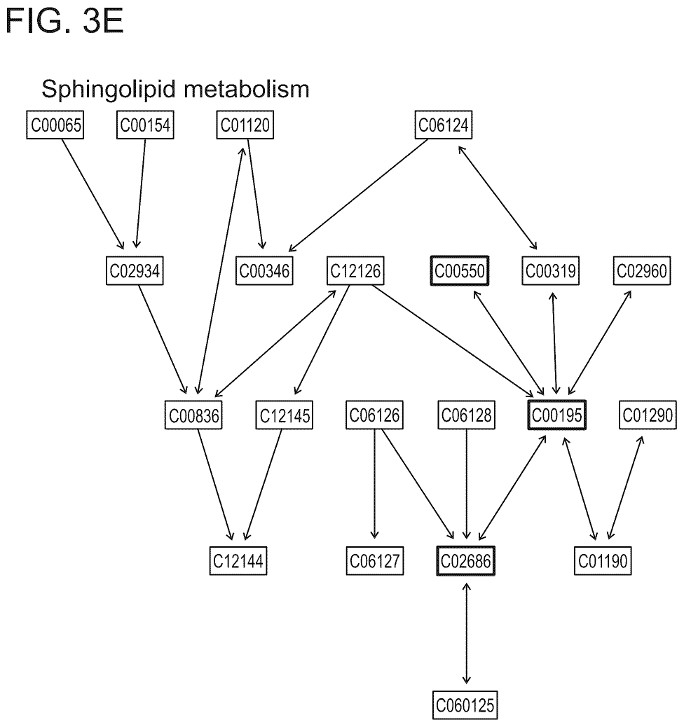

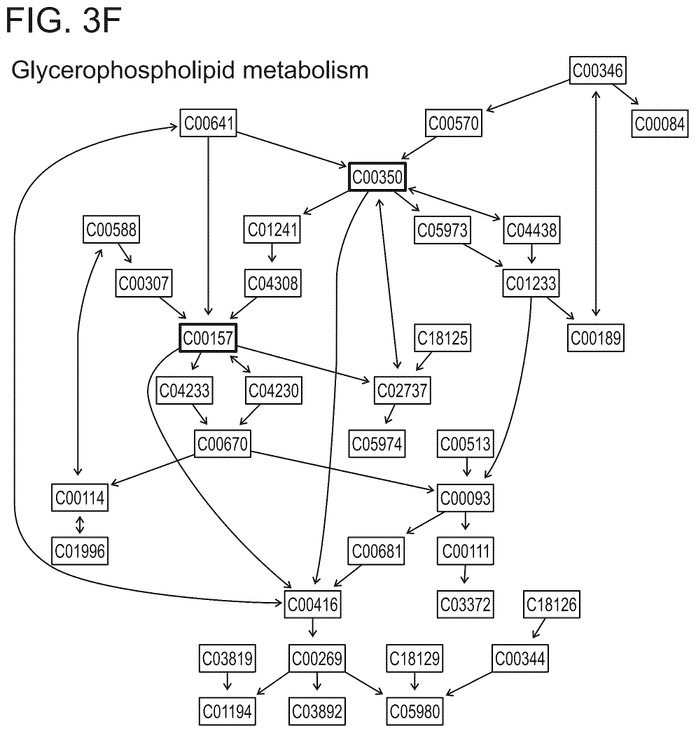

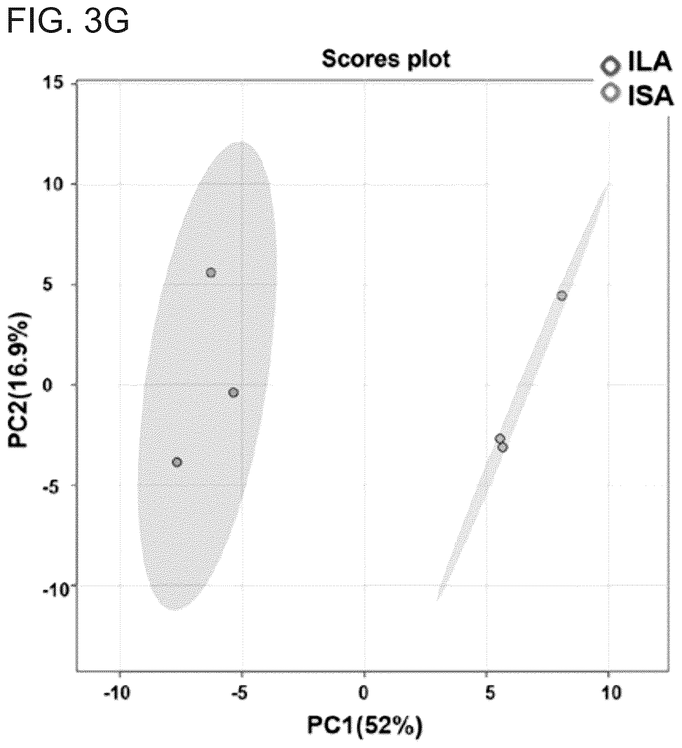

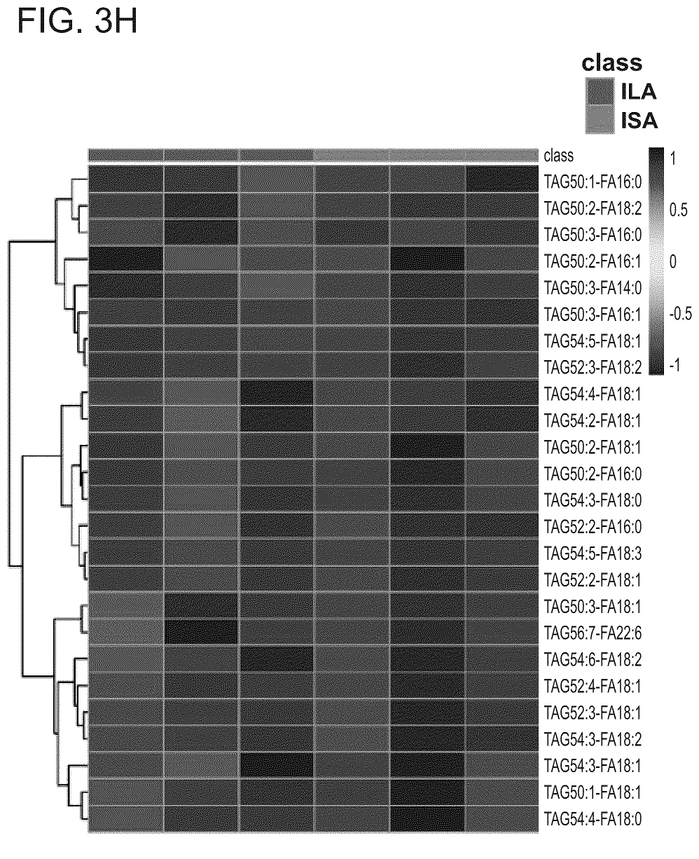

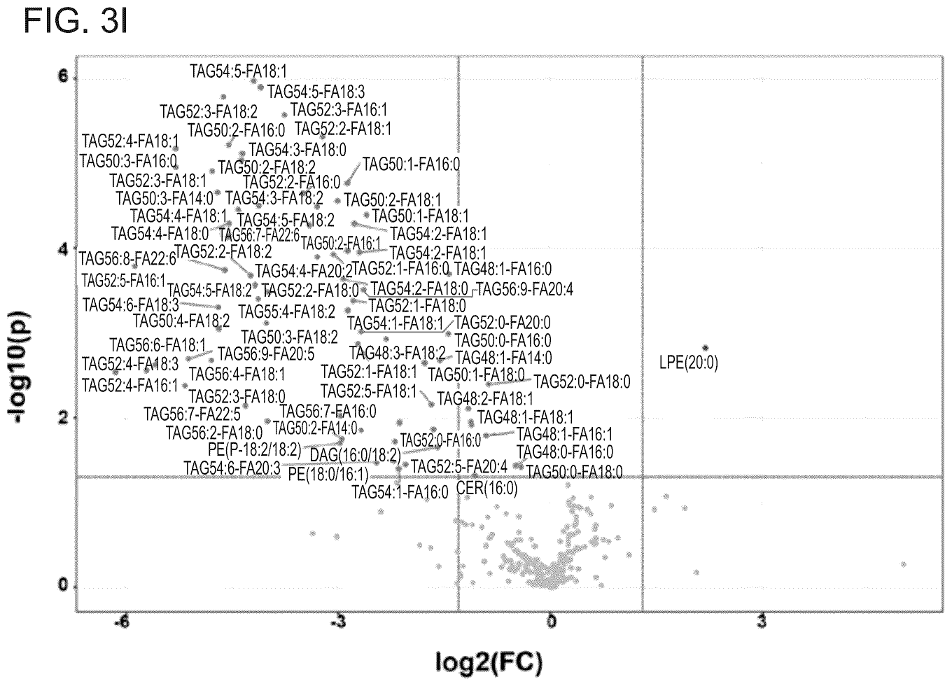

FIG. 12A, FIG. 12B, FIG. 12C, FIG. 12D, and FIG. 12E show that the lipidome of L-adipomes is distinct from that of S-adipomes; the figure is related to FIG. 3. FIG. 12A is a bar graph showing differentially abundant lipid molecule species (in nmol) including cholesterol, total FFA, DAG, TAG, and other lipid biomolecules in adipomes (CLA, CSA, ILA, and ISA) as quantitated by LC-MS/MS. FIG. 12B and FIG. 12D are PCA score plots of the lipid profiles from CSA and ISA (FIG. 12B) and CLA vs. CSA (FIG. 12D) showing clusters of two distinct groups. FIG. 12C and FIG. 12E are heatmaps showing clustering analysis of the top 25 differentially abundant lipid molecule species (with lowest FDR-adjusted p-values) based on z-scores for the normalized, transformed and scaled data between CSA and ISA (FIG. 12C), and CLA and CSA (FIG. 12E). For class name, red represents CLA or ILA and green represents ILA or ISA. For the abundance of each lipid, red represents high and blue represents low. The plotted values represent the mean, n=3 per group. Abbreviations: CLA, control large-adipomes; ILA, infected large-adipomes; CSA, control small-adipomes; ISA, infected small-adipomes; FFA, free fatty acides; DAG, diacylglycerols; TAG, triacylglycerols; CE, cholestyerol esters; CER, ceramides; HCER, hecosylceramide; LPE, lysophosphatidylethanolamine; LPI, lysophosphatidylinositol; MAG, monoacylglycerol; PC, phosphatidylcholine; PE, phosphatidylethanolamine; PE(O), alkenylphosphatidylcholine; PE(P), alkylphosphatidylethanolamine; PG, phosphatidylglycerol; PI, phosphatidylinositol; PS, phosphatidylserine; and SM, sphingomyelins.

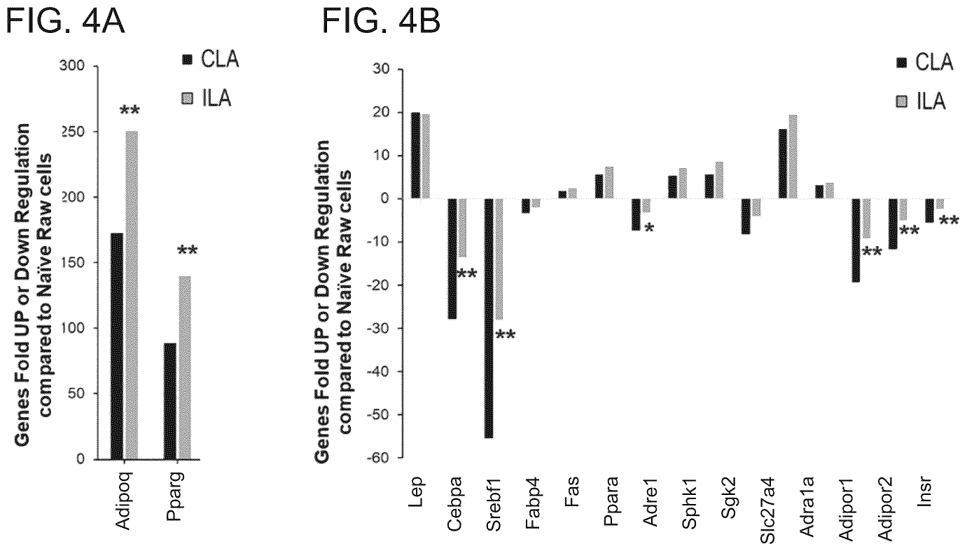

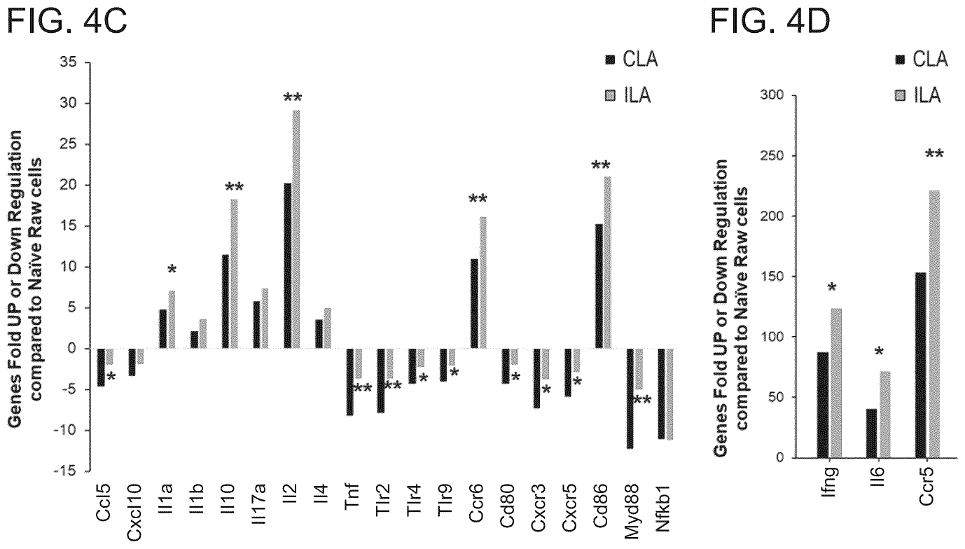

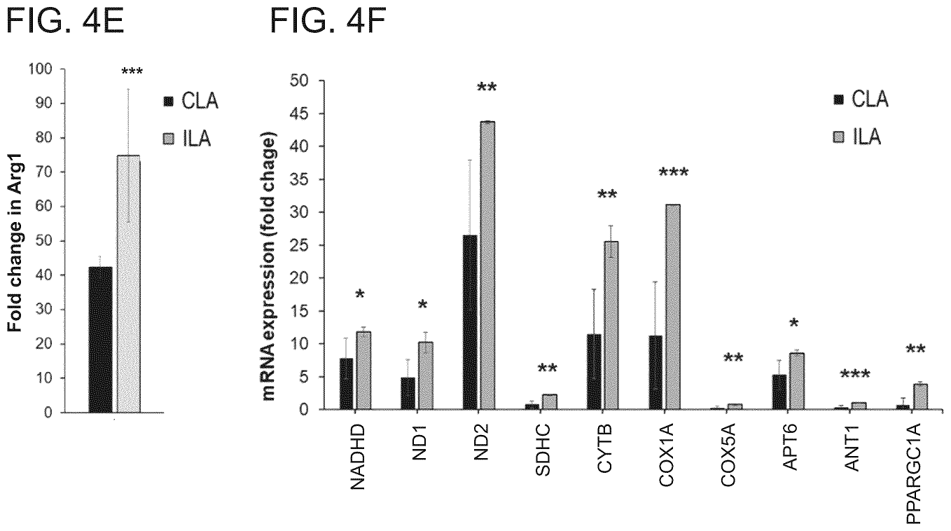

FIG. 13A and FIG. 13B show that adipomes regulate gene expression in cultured RAW macrophages; the figure is related to FIG. 4. FIG. 13A shows a custom RT2 profiler qPCR array plate design with gene names. FIG. 13B shows fold-regulation (up or down) of genes based on mRNA fold change values (normalized to Hprt) in macrophages treated with CLA, ILA, CSA, or ISA for 48 hours and compared to untreated macrophages as demonstrated by custom RT2 Profiler qPCR array analysis. Fold change was calculated using Qiagen data analysis spreadsheet.

FIG. 14A, FIG. 14B, FIG. 14C, FIG. 14D, FIG. 14E, and FIG. 14F show that adipomes regulate immunometabolic signaling and macrophage polarization; the figure is related to FIG. 4. FIG. 14A and FIG. 14B show fold change expression of Arg1 mRNA transcripts (normalized to Gapdh) in macrophages treated with CLA, ILA, CSA, or ISA when compared to untreated macrophages (FIG. 14A) and compared between different adipome treatments (FIG. 14B) as demonstrated by qPCR analysis (n=3). FIG. 14C shows fold change in mRNA transcript levels of genes involved in mitochondrial oxidative phosphorylation (normalized to Gapdh) in macrophages treated with CLA, ILA, CSA or ISA and compared to untreated macrophages as demonstrated by qPCR analysis (n=3). FIG. 14D, FIG. 14E, and FIG. 14F show fold change in mRNA transcript levels of genes involved in mitochondrial oxidative phosphorylation (normalized to Gapdh) in macrophages treated with ILA and ISA (FIG. 14D), CSA and ISA (FIG. 14E) and CLA and CSA (FIG. 14F) as demonstrated by qPCR analysis (n=3). Data are represented as mean±SEM. (*p<0.05; **p≤0.01; and ***p≤0.001).

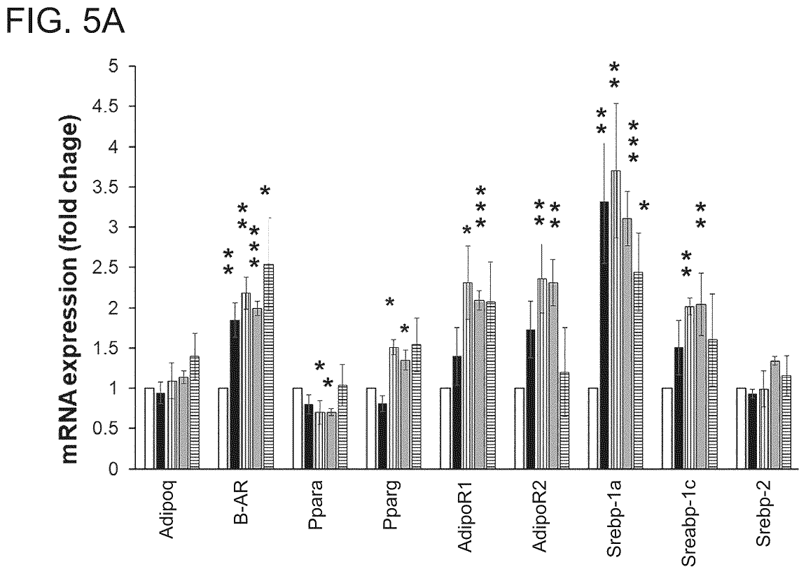

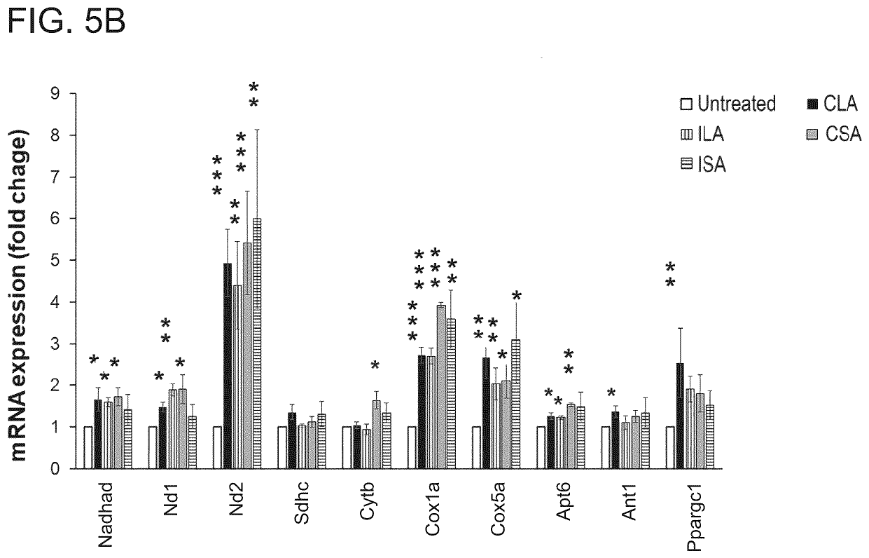

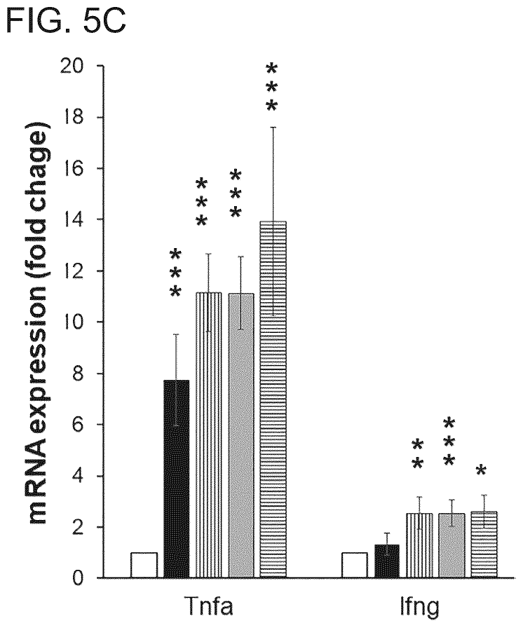

FIG. 15A, FIG. 15B, FIG. 15C, FIG. 15D, FIG. 15E, and FIG. 15F show that adipomes play a major regulatory role in cardiomyocytes functioning. This figure is related to FIG. 5. FIG. 15A, FIG. 15B, FIG. 15C, FIG. 15D, FIG. 15E, and FIG. 15F show fold changes in mRNA transcript levels of genes involved in adipogenic and lipogenic signaling (FIG. 15A and FIG. 15D); mitochondrial oxidative phosphorylation (FIG. 15B and FIG. 15E); and inflammatory signaling (FIG. 15C and FIG. 16F (normalized to Gapdh) in murine primary cardiomyocytes treated with adipomes for 48 hours and compared between control large adipomes (CLA) versus infected large adipomes (ILA)(FIG. 15A, FIG. 15B, FIG. 15C) and control large adipomes (CLA) versus control small adipomes (CSA) (FIG. 15DF, FIG. 15E, FIG. 15F), as demonstrated by qPCR analysis (n=3). Data are represented as mean±SEM (*p<0.05; **p≤0.01; and ***p≤0.001)

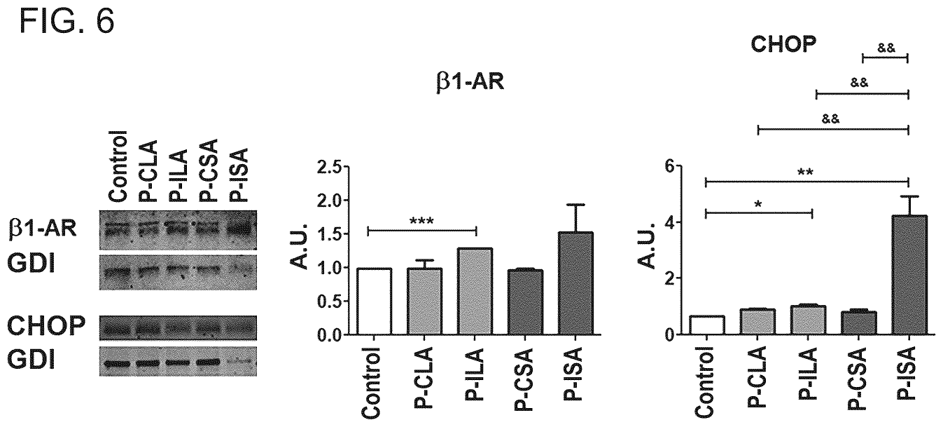

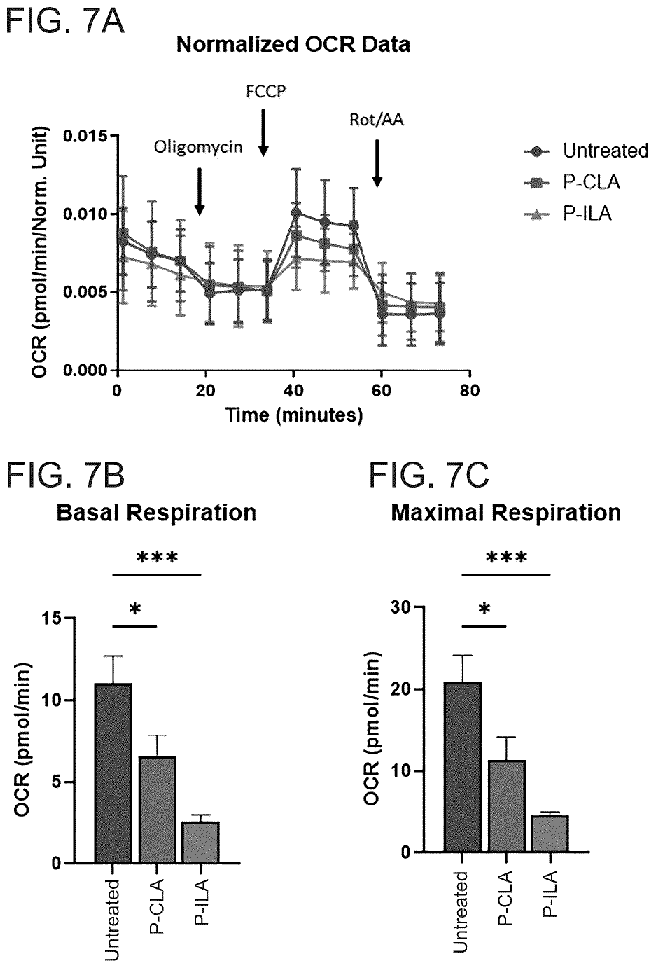

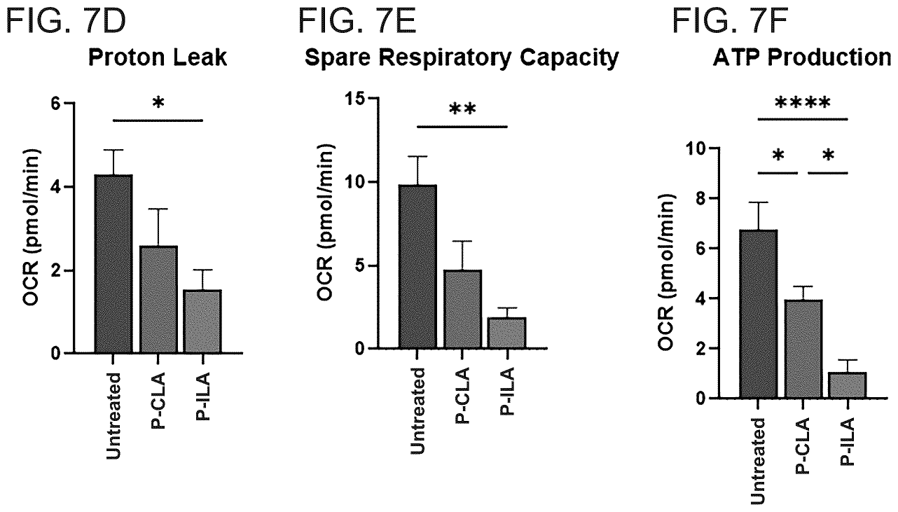

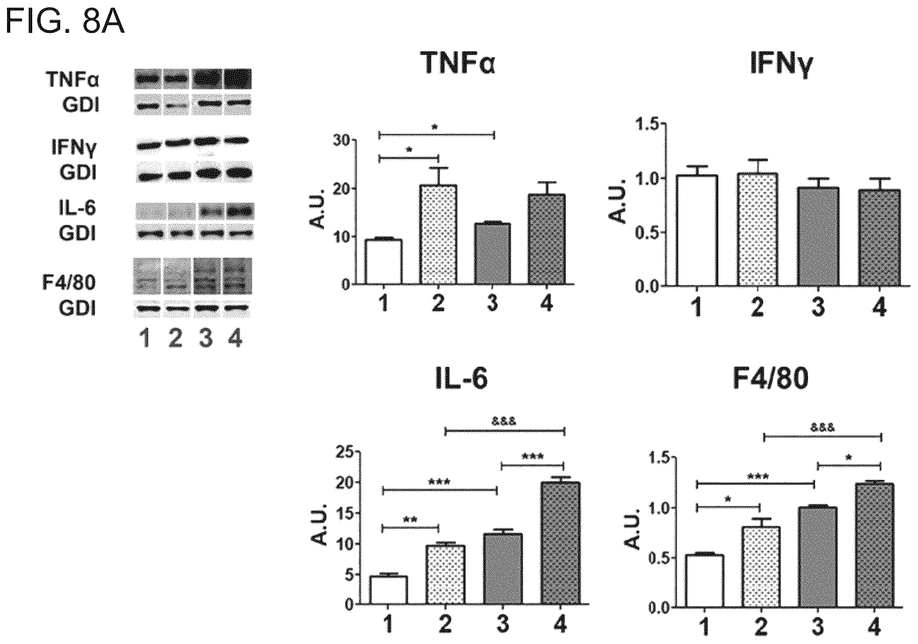

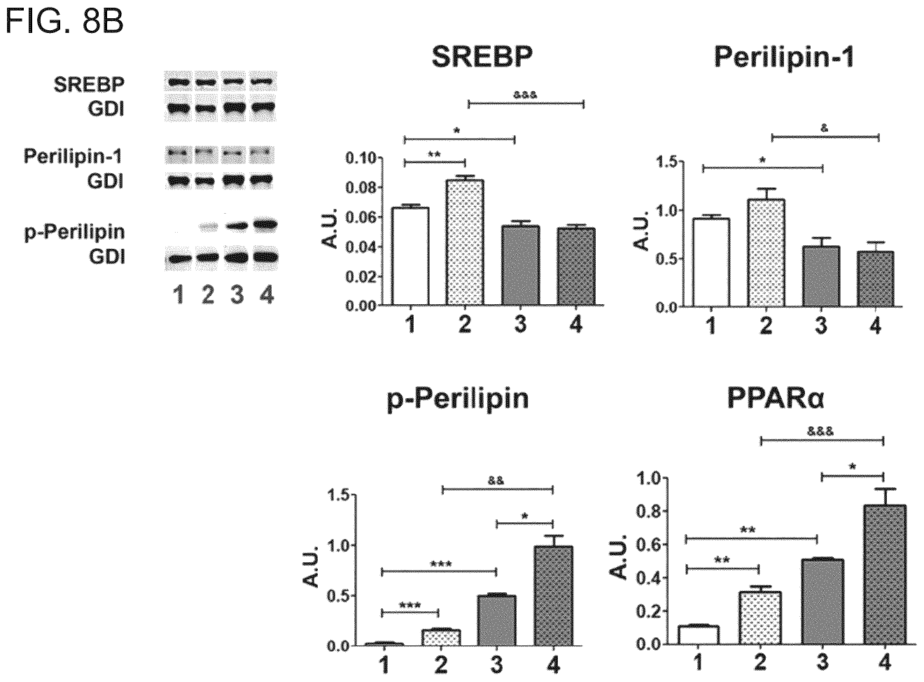

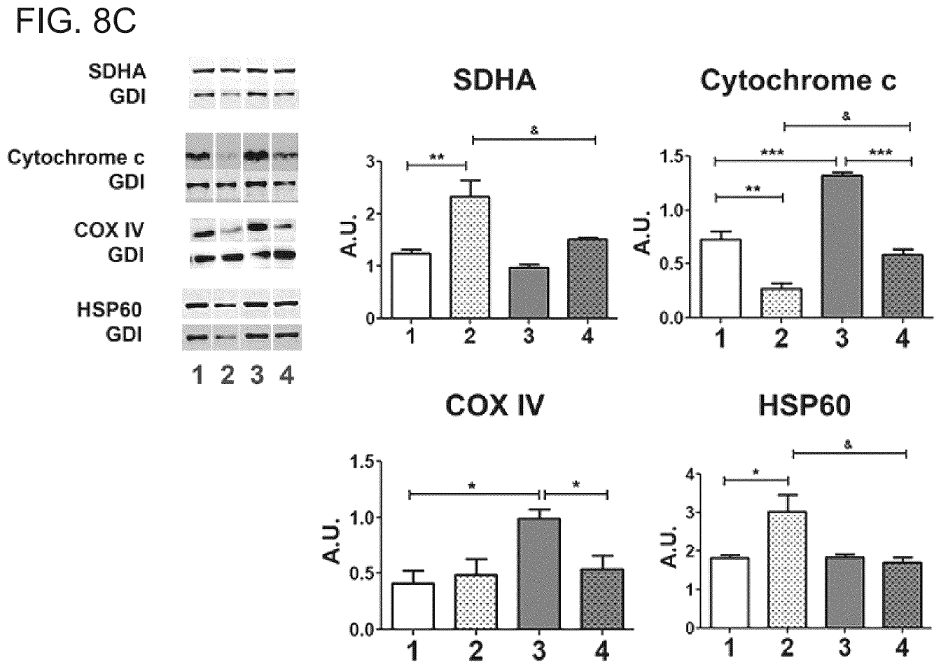

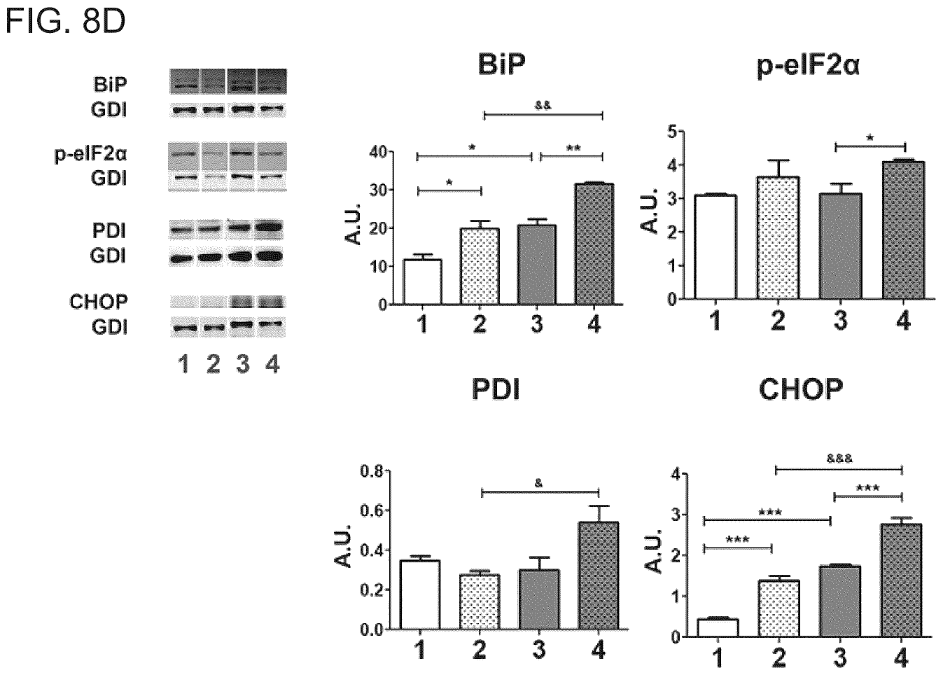

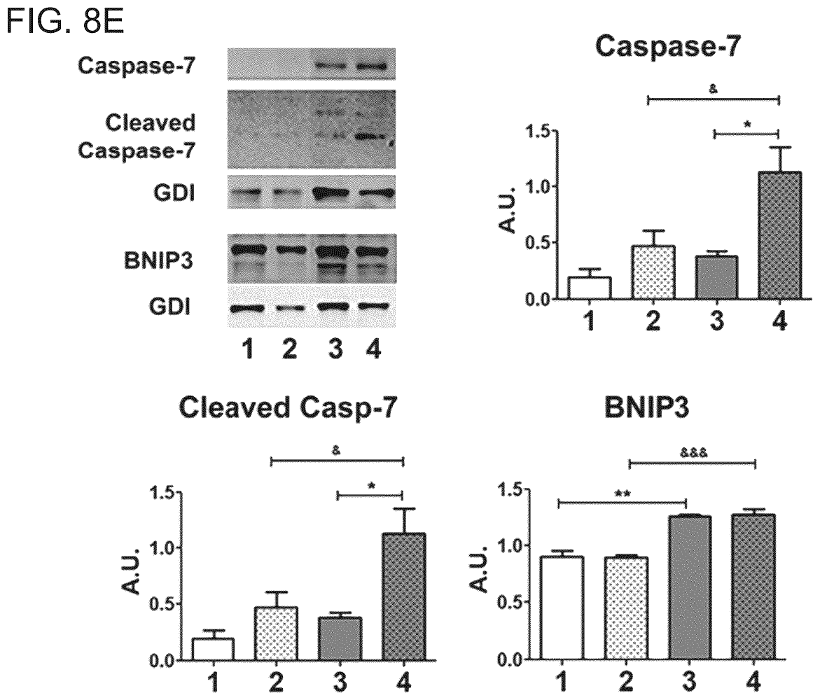

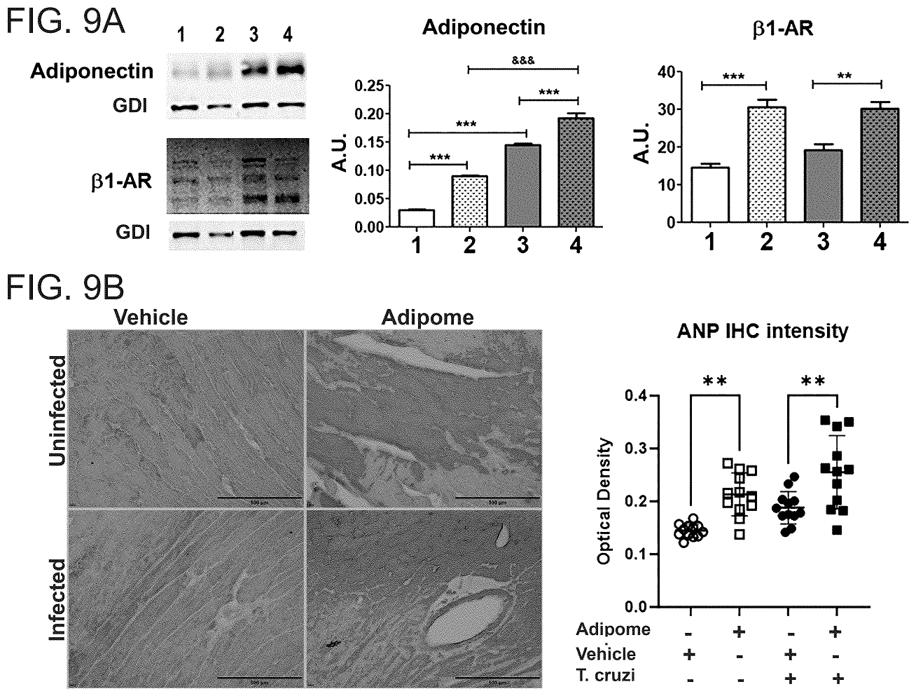

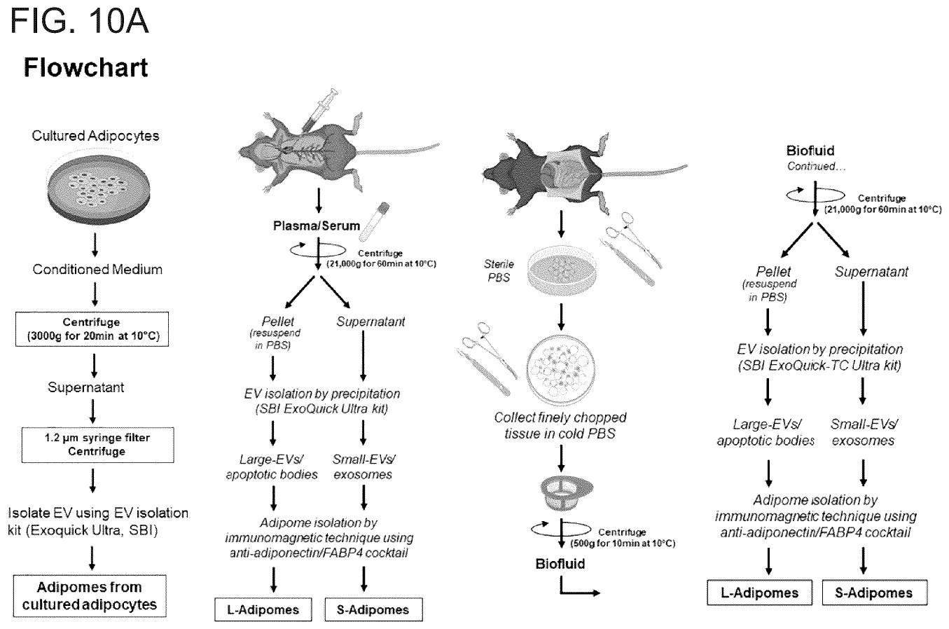

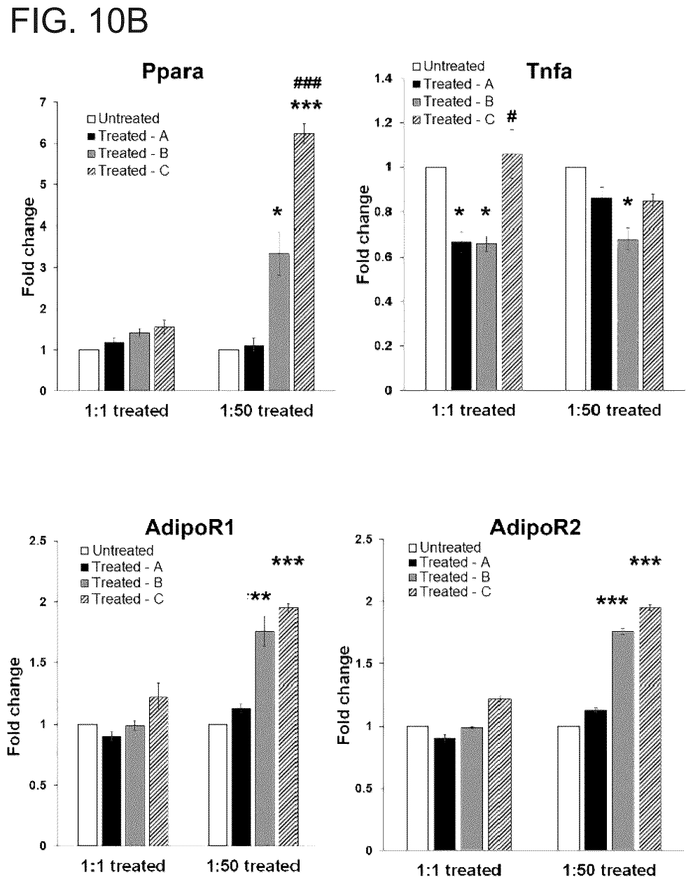

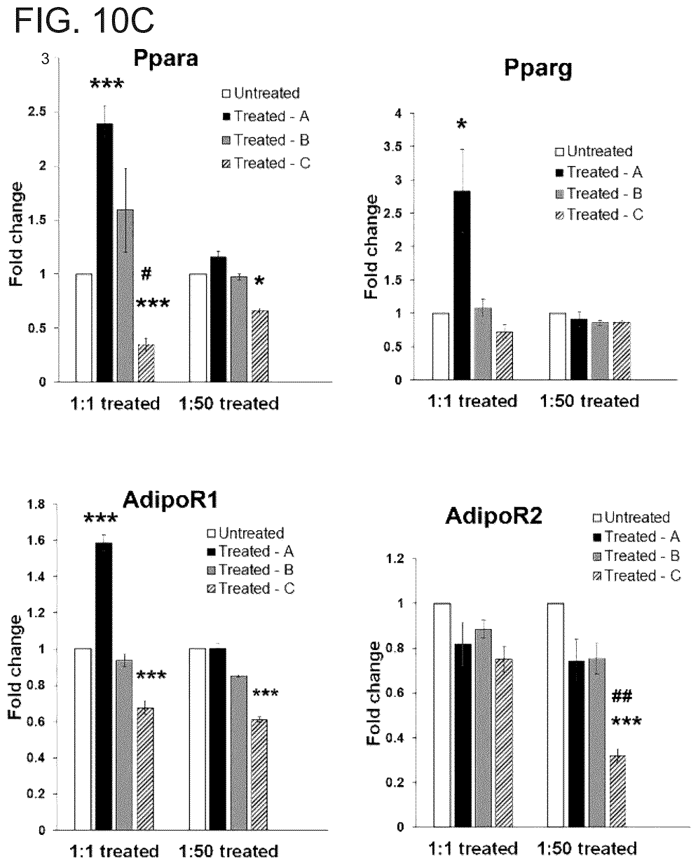

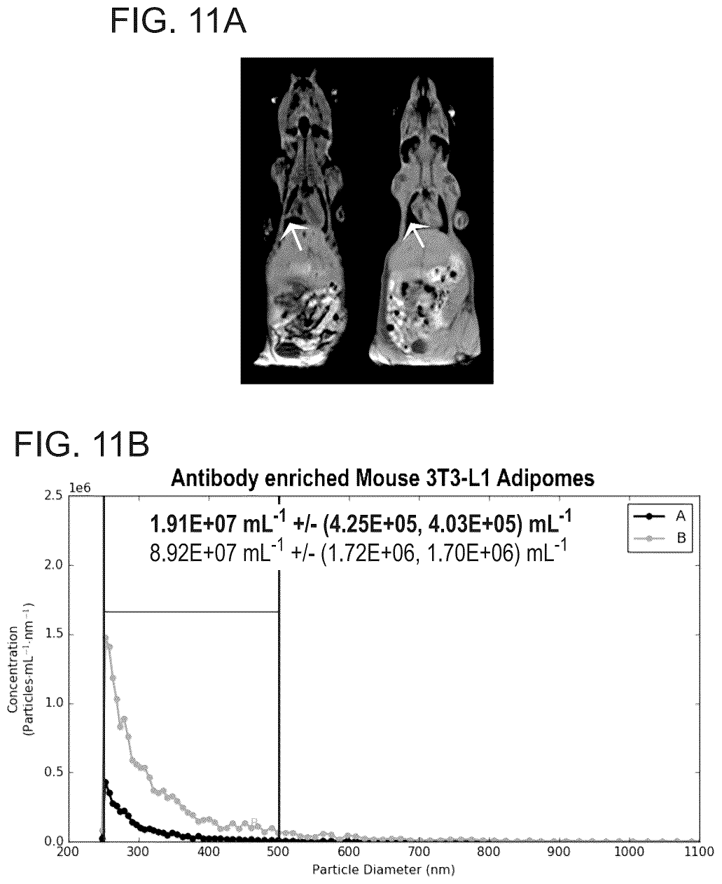

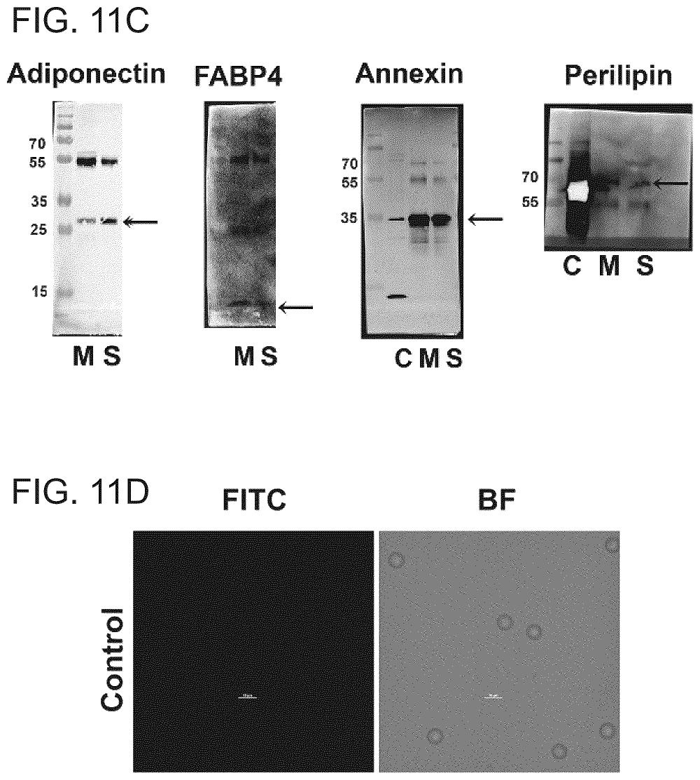

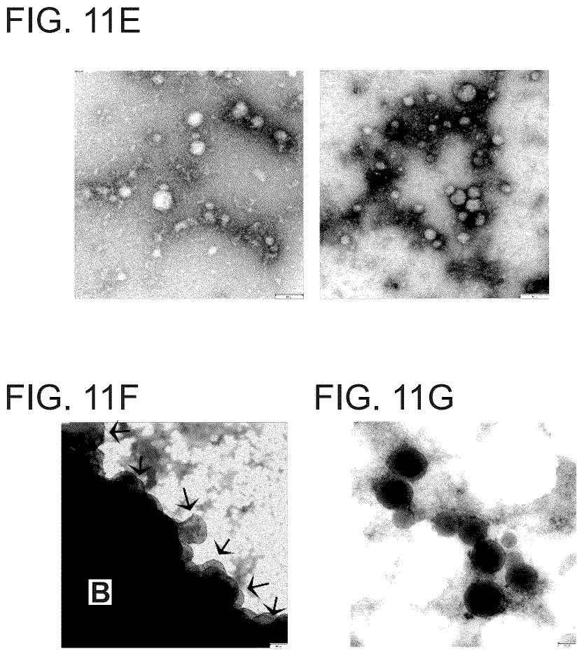

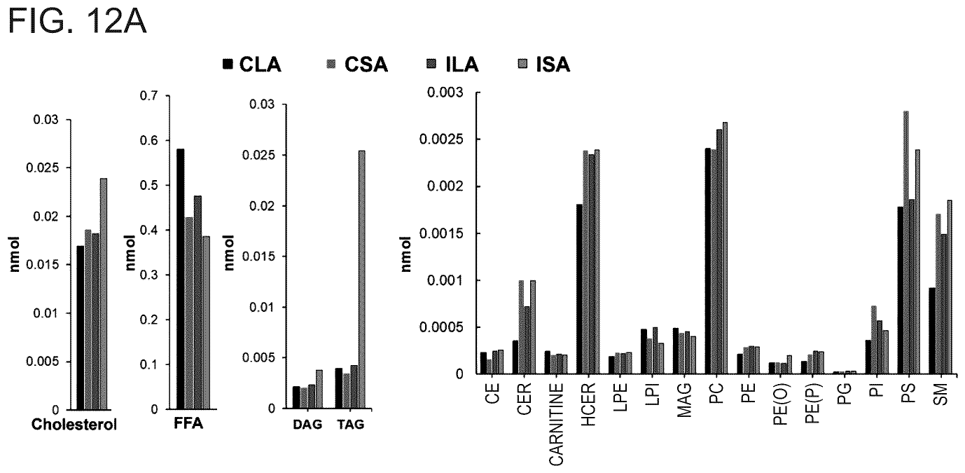

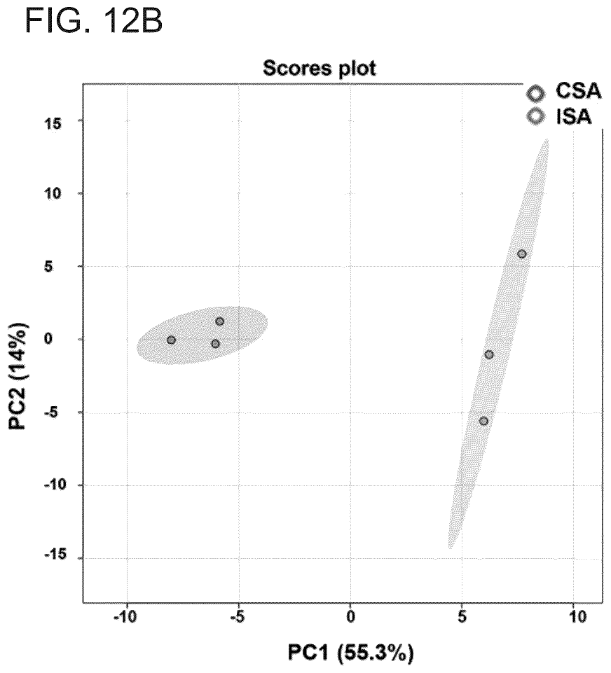

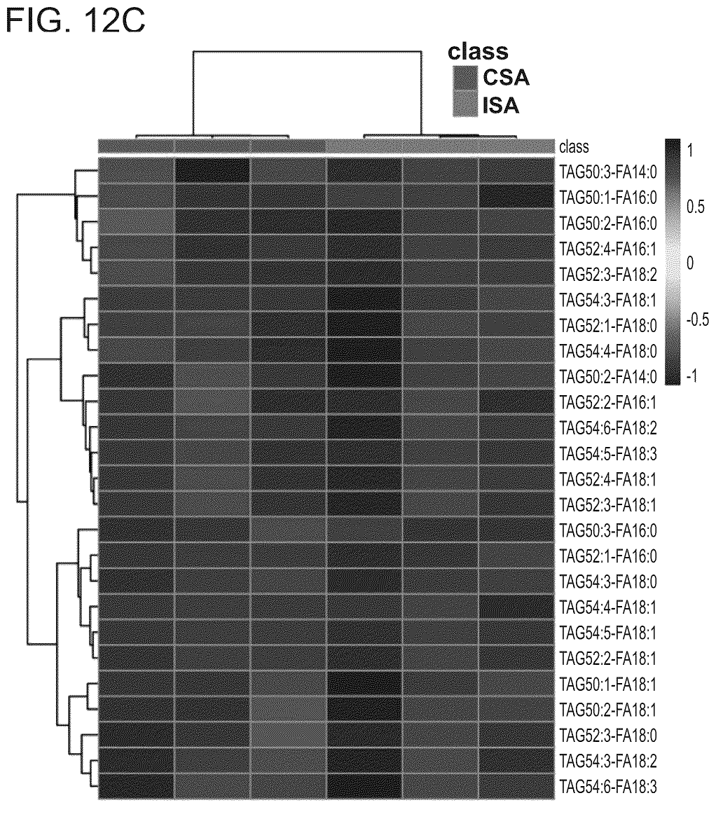

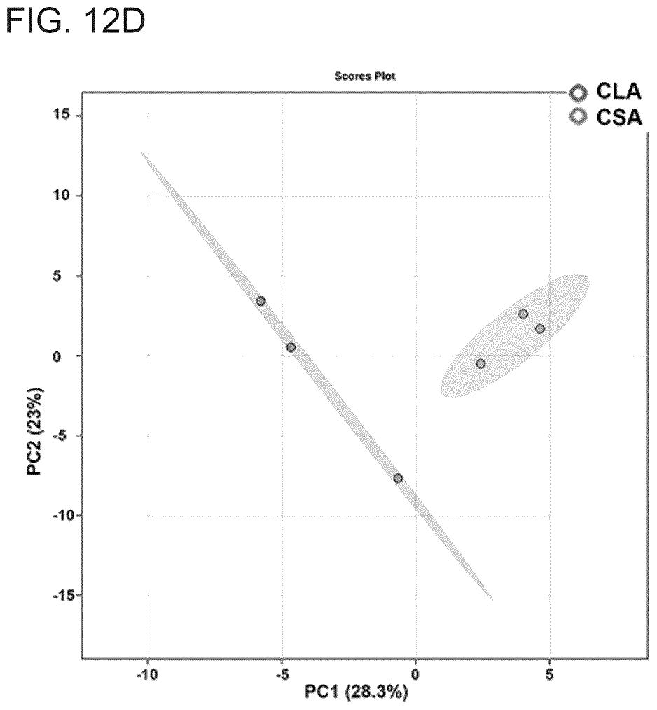

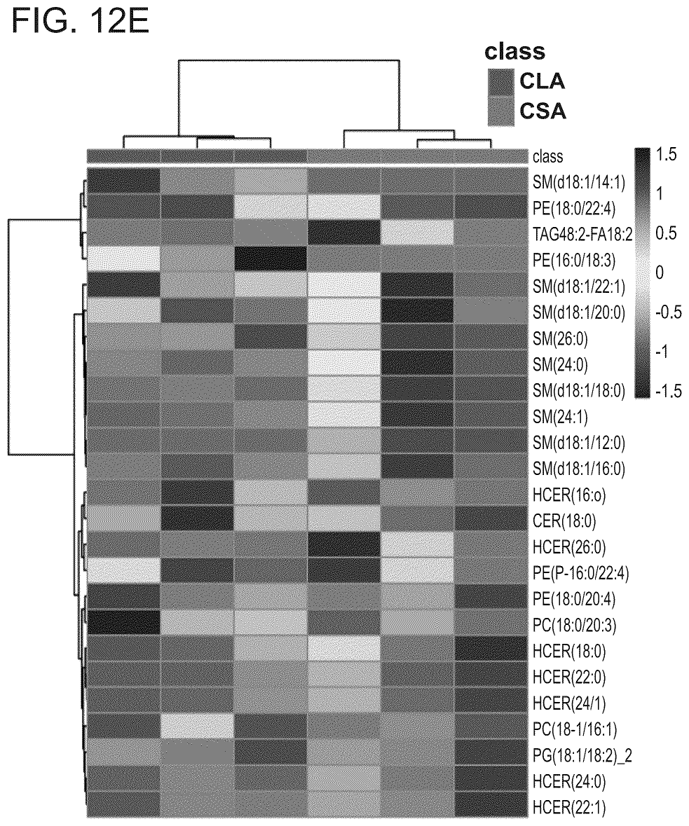

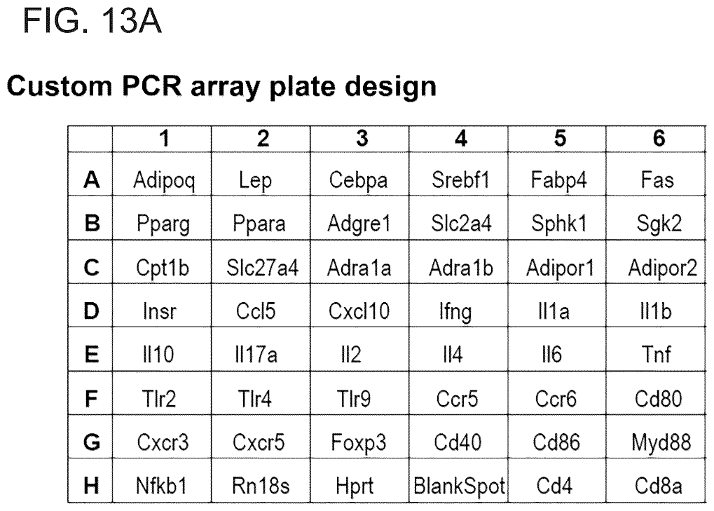

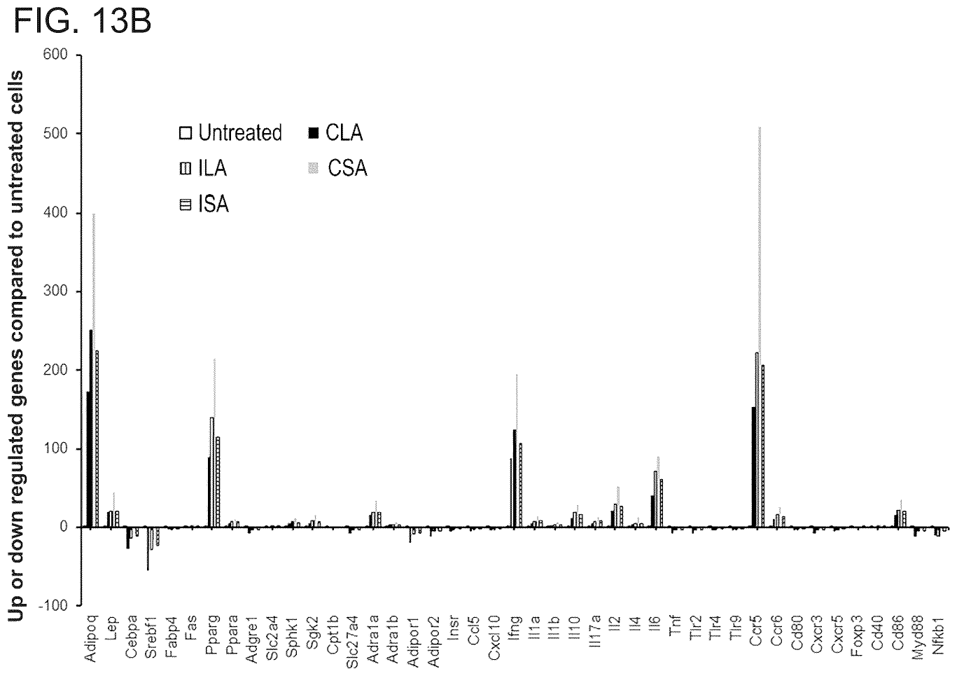

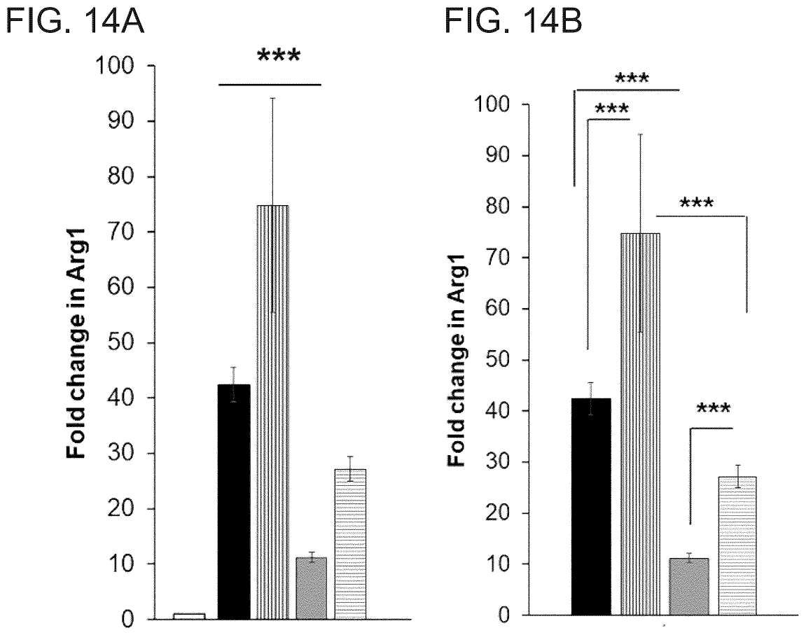

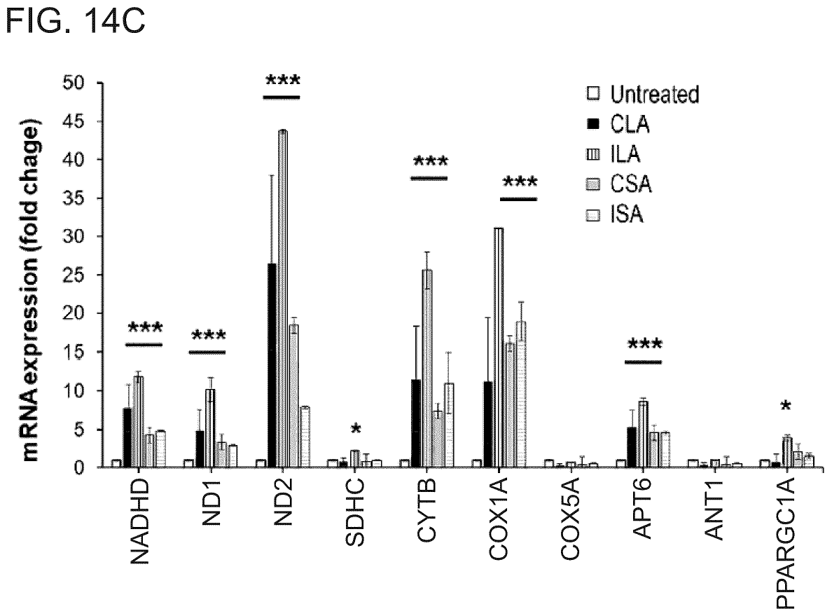

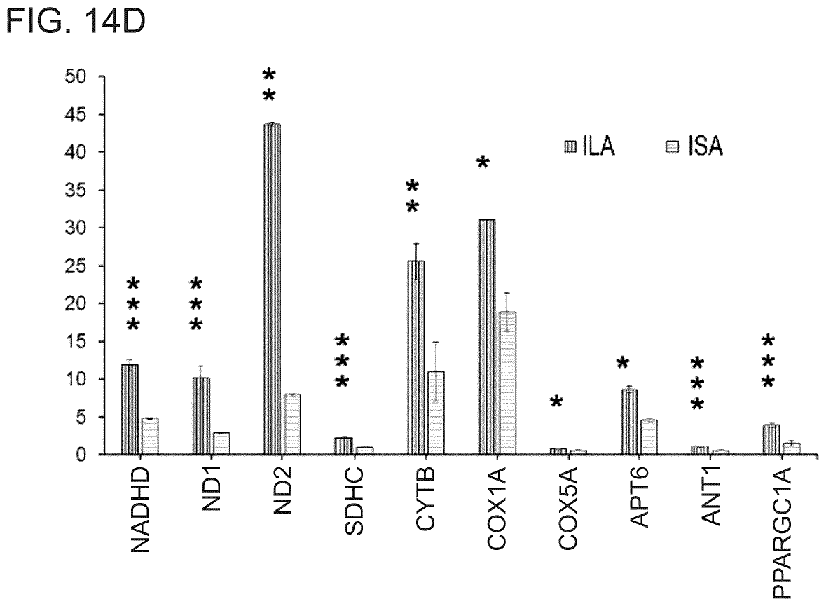

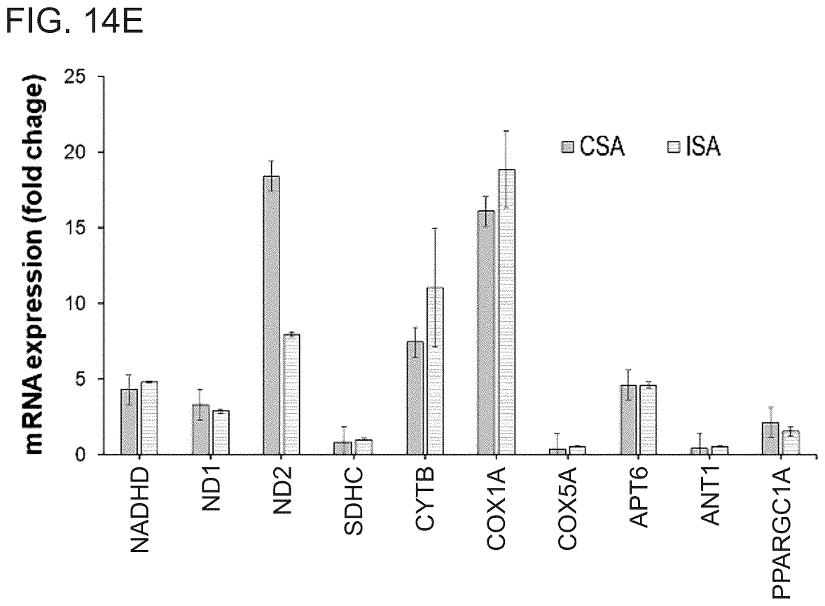

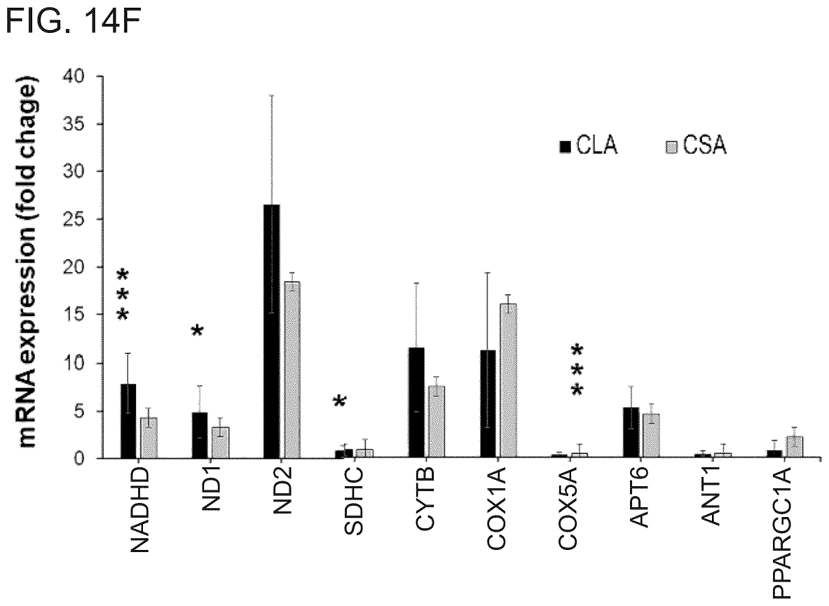

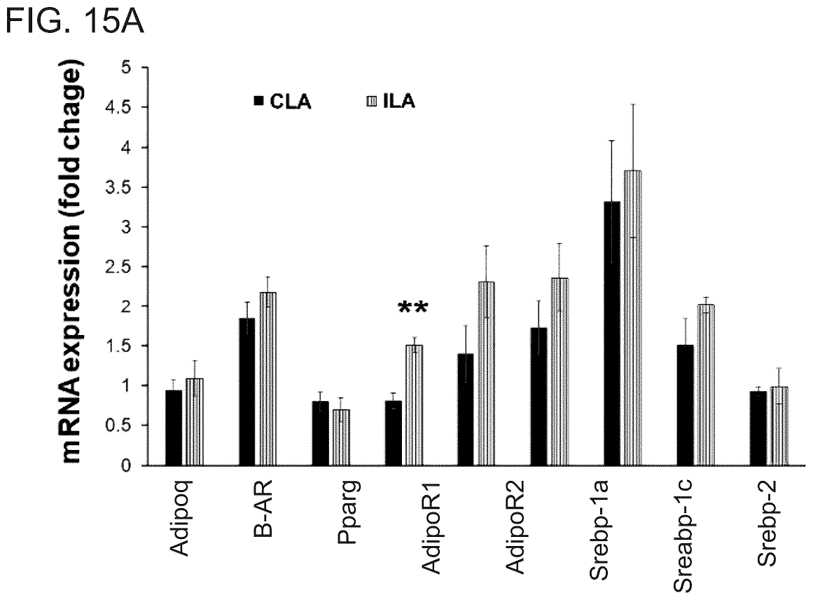

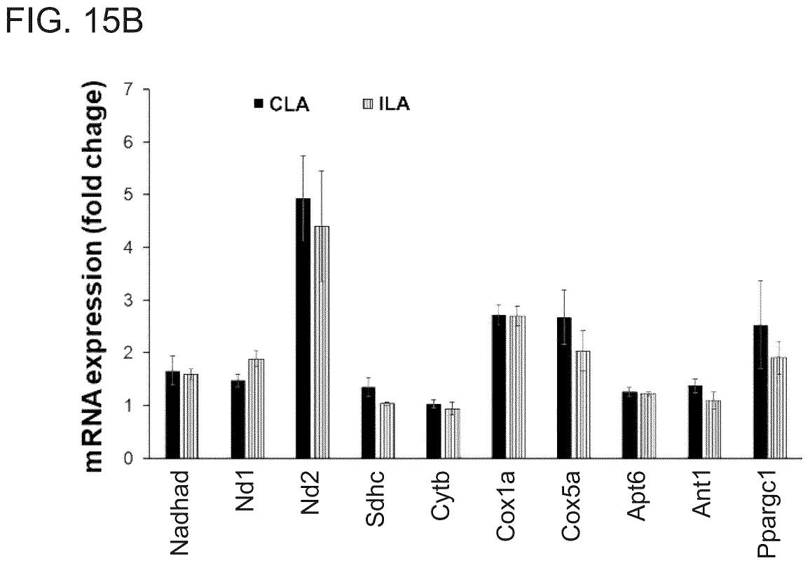

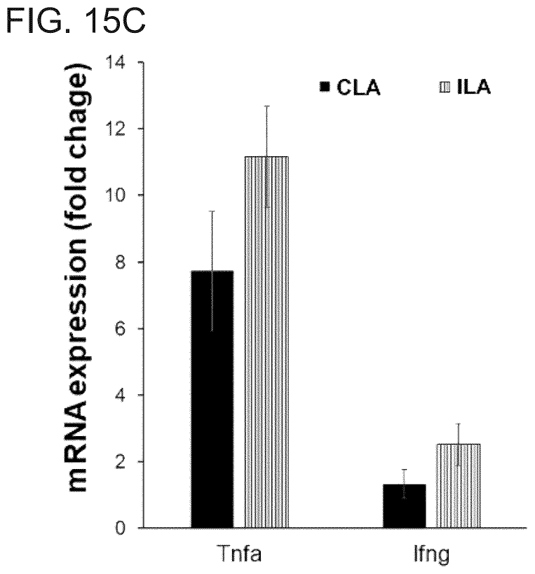

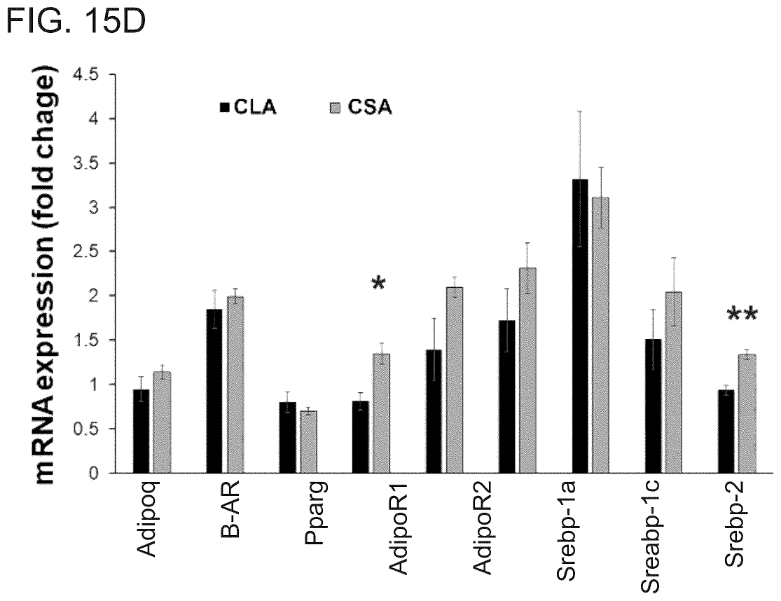

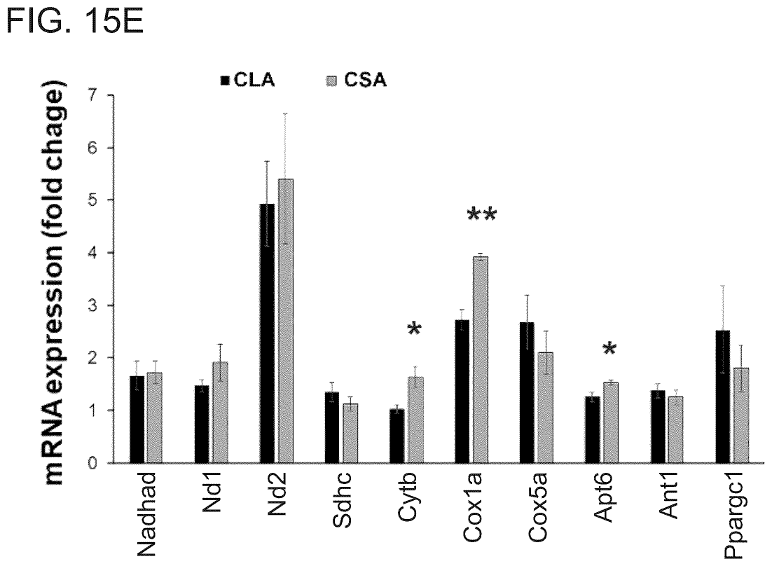

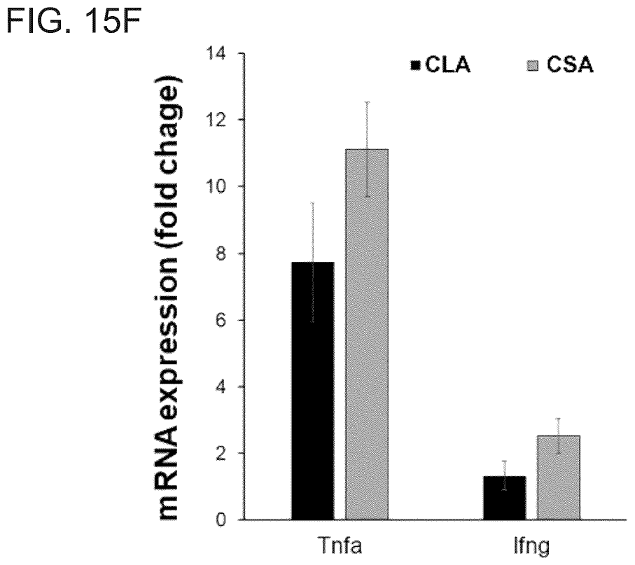

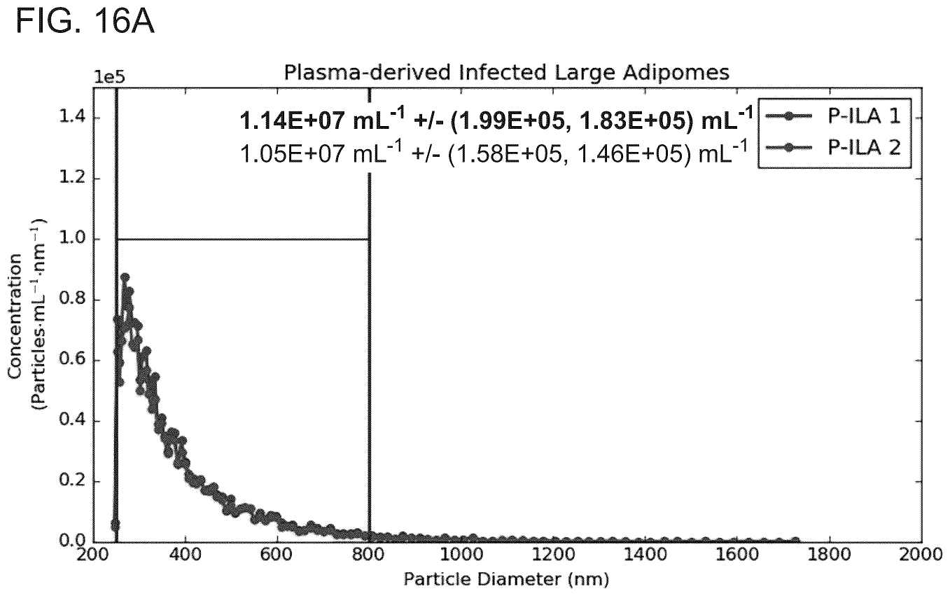

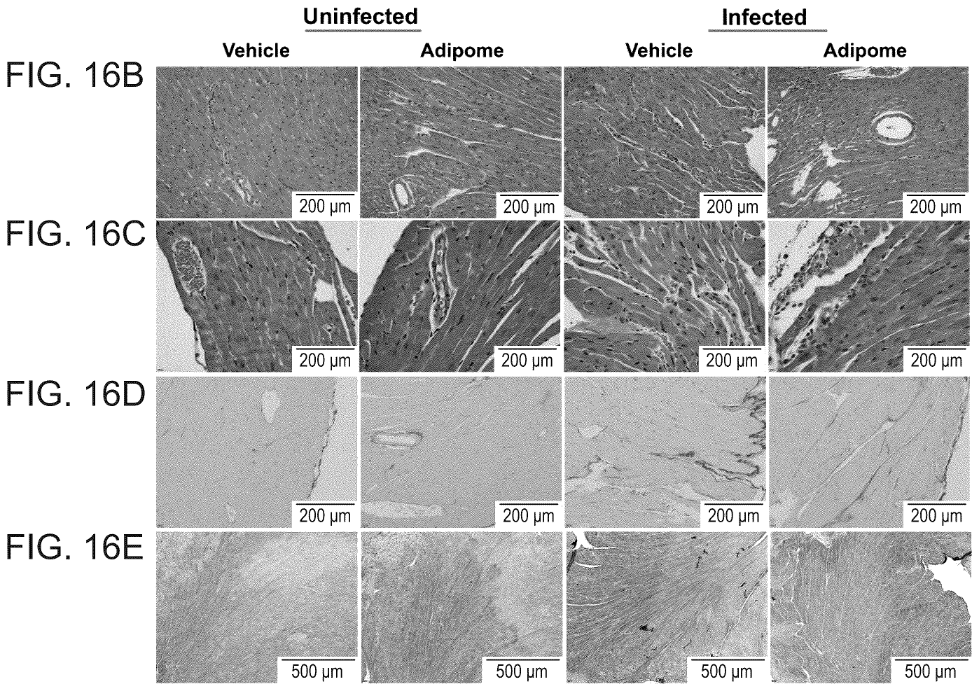

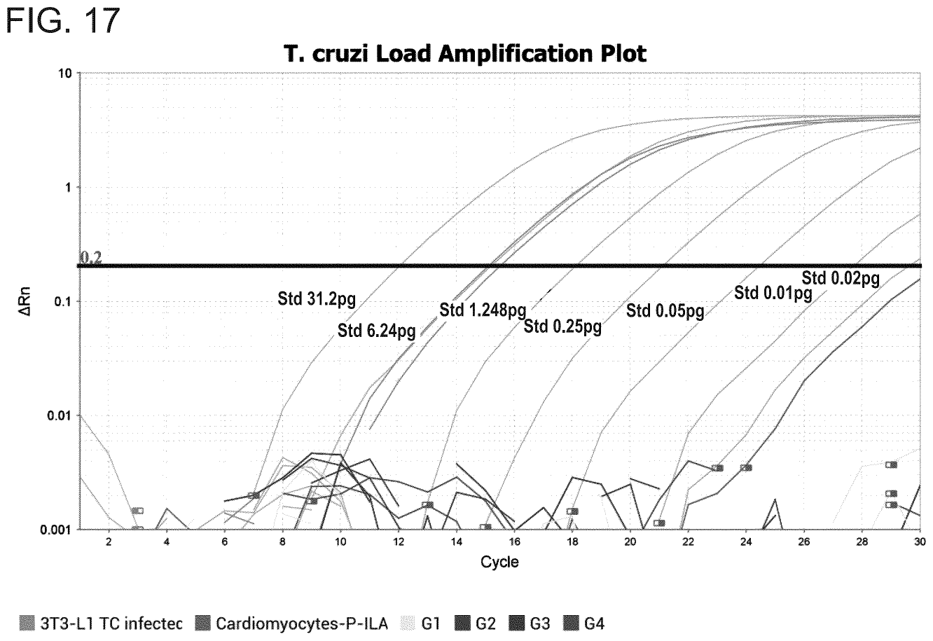

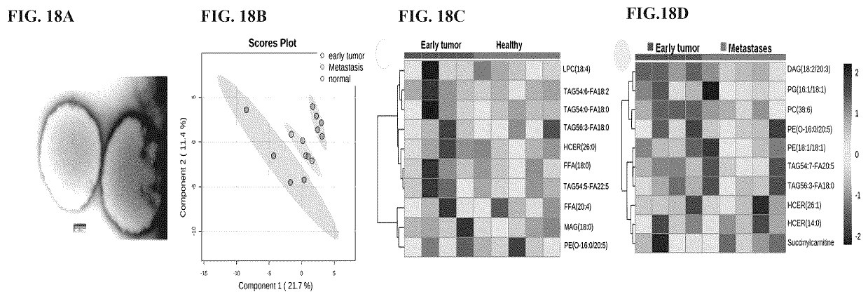

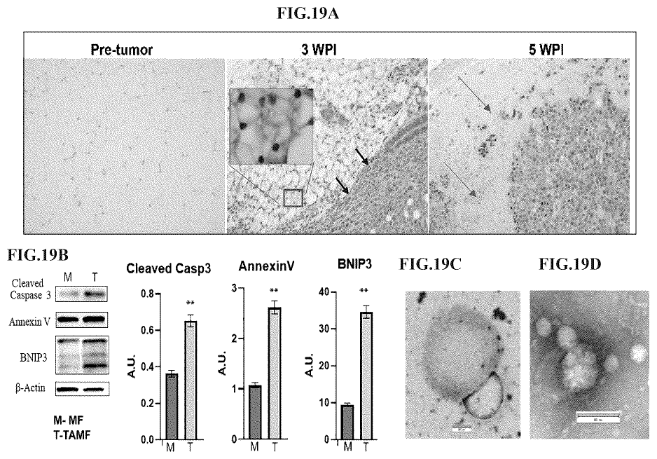

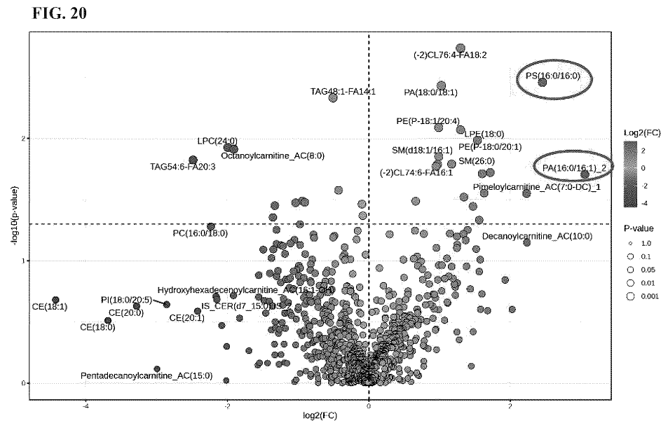

FIG. 16A, FIG. 16B, FIG. 16C, FIG. 16D, and FIG. 16E show that P-ILA increases cardiomyopathy risk in post-acute CD mice. This figure is related to FIG. 6, FIG. 7, and FIG. 8. FIG. 16A shows plasma-derived infection-associated large adipome (P-ILA) size distribution and absolute concentration as determined by Spectodyne nCS1 with a C-2000 microfluidic cartridge. Inset: P-IL-1 (blue) and P-TLA-2 (green) represent plasma-derived large adipomes from two different samples. FIG. 16B and FIG. 16C show H & E stained sections of vehicle- and P-ILA-injected heart tissue from uninfected and T. cruzi-infected mice. Representative images of myocardium (FIG. 16B) at 20× magnification, and right ventricle (RV) (FIG. 16C) at 40× magnification. FIG. 16D shows representative images of vehicle—and P-ILA-injected heart tissue from uninfected and T. cruzi-infected mice showing fibrosis as indicated by Picrosirius Red Polarization (PSRP) staining at 20× magnification. FIG. 16E shows representative immunohistochemistry (IHC) images of vehicle- and P-ILA-injected heart tissue from uninfected and T. cruzi-infected mice stained for TNF-α and imaged at 10× magnification.

FIG. 17 shows that adipomes are not reservoirs for T. cruzi parasites. This figure is related to FIG. 7, FIG. 8, FIG. 9 and Table 2. Standard curve PCR analysis revealed no trace of T. cruzi specific gene amplification I the heart tissue extracts of both P-ILA-treated uninfected mice (G2) and vehicle/P-ILA-treated post-infected (45 DPI) mice (G3 and G4). The qPCR analysis demonstrated the amplification of T. cruzi specific genes in cDNA made from 3T3-L1 adipocytes infected with T. cruzi (72 h, 1:5 MOI; positive control), while no gene amplification was observed in cDNA made from cardiomyocytes treated with P-ILA (72 h, 1:20 cell to adipome ratio). DNA dilutions from T. cruzi trypomastigotes were used as standards. G1, uninfected mice with vehicle treatment; G2, uninfected mice with P-ILA treatment; G3, infected mice with vehicle treatment; and G4, infected mice with P-ILA treatment.

FIG. 18A, FIG. 18B, FIG. 18C, and FIG. 18D show that adipomes isolated from the plasma of women with breast cancer (BC) and their lipid cargoes differ in women with localized, early-stage cancer vs metastatic disease. FIG. 18A is a Transmission Electronic Microscopy (negative) image of adipomes from plasma from a BC patient. Mean adipome size is 500 nm (bar-100 nm). FIG. 18B is a PCA score plot of lipids with samples colored according to their respective groups (pink dots-early breast tumor; green dots-metastases; blue dots-healthy). The principal components showed that the PC1 accounted for 21.7% of the total variance and the PC2 accounted for 11.4%. FIG. 18C is a heatmap showing that lipidomic profiles of the top 10 lipids of adipomes from plasma of early tumor subjects (red bar) differs from that of healthy subjects (green bar). FIG. 18D is a heatmap showing that lipidomic profiles of the top 10 lipids of adipomes from plasma of early tumor subjects (red bar) differs from those patients with metastatic BC (green bar). (n=4-5/group).

FIG. 19A, FIG. 19B, FIG. 19C and FIG. 19D show loss of adipocytes in the tumor microenvironment (TME) and associated release of large adipomes. We utilized a syngeneic E0771/C57BL/6 murine breast cancer model (8 weeks old, n=16), which demonstrated metastatic progression by 3 weeks post-tumor implantation (WPI). FIG. 19A is a histological analysis, which revealed that the mammary fat pad (left panel), initially composed of mature adipocytes, progressively transitioned into fibrotic tissue by 5 WPI (right panel, red arrow), concurrent with a marked loss of adipocytes. At 3 WPI (middle panel), H&E staining of tumor-associated mammary fat (TAMF) showed signs of adipocyte degradation (adipolysis) at the invasive tumor front (black arrow). The inset highlights a magnified view of adipose tissue adjacent to the tumor, illustrating adipocyte breakdown. FIG. 19B is a Western blot analysis, which demonstrated increased expression of apoptotic markers-cleaved caspase-3, annexin V, and Bnip3 (a Bcl2-interacting protein), in TAMF at 3 WPI relative to pre-tumoral mammary fat (MF). Protein levels were normalized to β-actin (n=3; p≤0.01). FIG. 19C and FIG. 19D are Transmission Electron Micrographs of L-adipomes, FIG. 19C) and S-adipomes, FIG. 19D. Transmission electron microscopy (TEM) confirmed the successful isolation of large adipomes and small adipomes from TAMF at 3 WPI. L-adipomes measured approximately 350 nm in diameter, whereas S-adipomes were ˜40 nm. Scale bar=80 nm.

FIG. 20 is a Volcano plot showing lipids of L-adipomes isolated from tumor-associated mammary fat (TAMF) vs. pre-tumoral mammary fat (MF). The Y-axis represents log 10p-value and the x-axis log 2FC. Among the enriched lipids were palmitic acid containing PA and PS (encircled) in TAMF-adipomes.

### SUMMARY OF THE INVENTION

According to one aspect, the present disclosure provides a method for early diagnosis of a subject at risk for a pathology comprising alterations in metabolic and inflammatory signaling in phagocytic and nonphagocytic target cells, the method comprising: selectively purifying from a population of various cell types that include adipocytes, a subpopulation of apototic adipocytes or dying adipocytes expressing adipocyte markers including adiponectin, FABP4 or both; isolating from the subpopulation of adipocytes including apoptotic adipocytes a population of adipocyte-specific extracellular vesicles (adipomes) derived from the population of total WAT-derived extracellular vesicles; enriching intact large adipomes (L-adipomes) from large EVs and small adipomes (S-adipomes) from small EVs that express the adipocyte markers adiponectin and FABP4 by positive selection employing adiponectin and FABP4 antibodies; extracting cargo of the L-adipomes and the S-adipomes and determining their lipid profile; comparing mRNA expression levels of the L-adipomes and S-adipomes from the subject to mRNA expression levels of a healthy control including adiponectin receptors AdipoR1 and AdipoR2; and determining a level of expression of one or more genes encoding a panel of proteins that function in lipolytic signaling, lipogenesis, mitochondrial signaling, inflammation or a combination thereof in target cells; wherein early diagnosis of the pathology can lead to improved outcome for the subject.

According to some embodiments of the method, a source of the adipomes population is plasma or white adipose tissue (WAT) comprising an adipocyte population including the subpopulation of apoptotic adipocytes or dying adipocytes and a non-adipocyte population comprising adipocyte precursor cells.

According to some embodiments, the L-adipomes are derived from large EVs comprising apoptotic bodies and macrovesicles and the S-adipomes are derived from small EVs comprising microvesicles and exosomes.

According to some embodiments, the pathology affects lipid metabolism, mitochondrial oxidative phosphorylation, inflammation, or a combination thereof.

According to some embodiments, the L-adipomes and S-adipomes derived from the adipocytes express adiponectin, FABP4, annexin V and perilipin.

According to some embodiments, size of the L-adipomes ranges from 200-400 nm and size of the S-adipomes ranges from 30-80 nm.

According to some embodiments, the panel of genes includes: a gene encoding a protein of lipolytic signaling comprising beta-2-adrenergic receptor (ADRB2) protein; a gene encoding a protein of lipogenesis comprising one or more of lipogenic SREBP1a and SREBP1c proteins; genes encoding a protein of mitochondrial signaling comprising one or more of NADHD, ND1, ND2, SDHC, CYTB, COX1A, COX5A, APT6, ANT1 or PGC-1a protein; and a gene encoding a protein of inflammatory signaling comprising TNFa, IFNg, or both proteins.

According to some embodiments, the pathology comprises polarization of a macrophage population derived from the subject at risk compared to a healthy subject. According to some embodiments, the pathology places at risk cardiomyocytes, cardiac fibroblasts or both. According to some embodiments, the pathology in an untreated subject progresses to hypertrophied cardiomyopathy, dilated cardiomyopathy and heart failure. According to some embodiments, the hypertrophied cardiomyopathy comprises cardiac remodeling and cardiomyocyte dysfunction.

According to some embodiments, a source of the pathology is an infection. According to some embodiments, the infection is a parasitic infection. According to some embodiments, the parasitic infection is an infection with Trypanosoma cruzi, a causative agent of Chagas disease.

According to another aspect, the present disclosure provides a method for modulating adipogenic signaling in a target cell population comprising: selectively purifying from a population of adipocytes a subpopulation of apototic adipocytes or dying adipocytes expressing adipocyte markers including adiponectin, FABP4 or both; isolating from the subpopulation of apoptotic and dying adipocytes a population of adipocyte-specific extracellular vesicles (adipomes) derived from the population of total WAT-derived extracellular vesicles, wherein size of the adipomes ranges from 200 nm to 1100 nm; enriching intact large adipomes (L-adipomes) from large EVs and small adipomes (S-adipomes) from small EVs that express the adipocyte markers adiponectin and FABP4 by positive selection employing adiponectin and FABP4 antibodies; extracting cargo of the L-adipomes and the S-adipomes and determining their cargo profile, wherein the cargo of the adipomes includes adipogenic mRNA; treating the population of target cells with the adipomes comprising the adipogenic mRNA cargo; and modulating gene expression of adiponectin and downstream genes regulated by adiponectin.

According to some embodiments of the method, a source of the adipomes population is plasma or white adipose tissue (WAT) comprising an adipocyte population including a subpopulation of apoptotic adipocytes or dying adipocytes and a non-adipocyte population comprising adipocyte precursor cells.

According to some embodiments, the L-adipomes are derived from large EVs comprising apoptotic bodies and macrovesicles and the S-adipomes are derived from small EVs comprising microvesicles and exosomes.

According to some embodiments, the target cell population comprises macrophages, fibroblasts or both.

According to some embodiments, the adipogenic genes comprise mRNA for Adipoq, Fabp4, and Pparg.

According to some embodiments, the downstream genes regulated by adiponectin include Ppara and adiponectin receptor R2 (AdipoR2).

### DETAILED DESCRIPTION OF THE INVENTION

### Definitions

As used herein and in the appended claims, the singular forms “a” “an”, and “the” include plural reference unless the context clearly dictates otherwise. Thus, for example, reference to a “peptide” is a reference to one or more peptides and equivalents thereof known to those skilled in the art, and so forth.

As used herein, the term “about” means plus or minus 10% of the numerical value of the number with which it is being used. Therefore, about 50% means in the range of 40%-60%.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer in one embodiment to A without B (optionally including elements other than B); in another embodiment, to B without A (optionally including elements other than A); and, in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps.

The term “adaptor” as used herein refers to nonenzymatic proteins that form physical links between members of a signaling pathway, particularly between a receptor and other signaling proteins. They recruit members of the signaling pathway into functional protein complexes.

The term “adipogenesis” as used herein refers to a cellular process characterized by heightened expression of adipogenic genes, which can subsequently modify the cell phenotype to resemble that of adipocytes. After the activation of specific transcription factors such as SREBP, C/EBPβ/δ, and PPARγ, along with the upregulation of genes such as adiponectin, Foxo1, and Fabp4, non-adipocyte cells, such as cardiomyocytes, may undergo a transformation, adopting adipocyte-like characteristics. Consequently, they can accumulate excessive lipids and may experience a loss of their original functionalities.

The term “adiponectin” as used herein a protein hormone that is produced by fat cells. Its physiological effects include the reduction of inflammation and atherogenesis (the formation of fatty deposits in the arteries) and enhancement of the response of cells to insulin. There are two subtypes of adiponectin receptors, AdipoR1 (375 aa residues, 42.4 kDa) and AdipoR2 (311 aa residues, 35.4 kDa). AdipoR1 signals are predominantly mediated via AMP-activated protein kinase (AMPK) activation. The N-terminal intracelluar of AdipoR1 binds to an adaptor protein containing a pleckstrin homology (pH) domain, a phosphotyrosine binding (PTB) domain, and a leucine zipper motif 1 (APPL1) to form an AdipoR1/APPL1 complex. Furthermore, Rab5 (a small GTPase) is attached to the PH domain of APPL1, and this enhances GLUT4 membrane translocation. On the other hand, AdipoR2 signals are mainly mediated via PPARα activation with an increase in the PPARα ligand and enhancement of the transcription of PPARα. [Hirako, S. Subchapter 48B—Adiponectin, in Handbook of Hormones (2d Ed.), Hironori Ando, Kazuyoshi Ukena, Shinji Nagata, Eds. Academic Press (2021), pp. 577-79].

The term “adipose cell” or “adipocyte” as used herein refers to the main cellular component of white adipose tissue. The main function of the adipocyte is to act as a buffer for the storage of excess fatty acids (FAs) as inert triacylglycerols (TAGs) in organelles termed lipid droplets (LDs) [Yang, A. and Motillo, EP. Biochem. J. (2020) 477 (5): 985-1008, citing Unger, R H and Scherer, PE. Trends in Endocrinology & Metabolism (2010) 21 (6): 345-52]. When energy demand is increased, TAGs are subsequently broken down into their constituent FAs and glycerol through the highly active and dynamic biochemical process of lipolysis. Adipocyte lipolysis is a dynamic regulatory process involving the assembly and disassembly of protein complexes on the surface of LDs. Upon stimulation, patatin-like phospholipase domain containing 2 (PNPLA2)/adipocyte triglyceride lipase (ATGL), the rate limiting enzyme for TAG hydrolysis, is activated by the interaction with its co-activator, alpha/beta hydrolase domain-containing protein 5 (ABHD5), which is normally bound to perilipin 1 (PLIN1). Negative regulators of lipolysis include G0/G1 switch gene 2 (GOS2) and PNPLA3 which interact with PNPLA2 and ABHD5, respectively. Once released, FAs have a variety of fates including oxidation for energy in the form of ATP, re-esterification back into TAGs [Id., citing Edens, N K et al. J. Lipid Res. (1990) 31 (8): 1423-31, and also functioning as signaling molecules [Id., citing Motillo, E P et al., J. Biol. Chem. (2012) 287 (30): 25038-48; Ong, K T et al. Hepatology (2011) 53 (10): 116-26; Zechner, R. et al. Cell Metabollism (2012) 15 (3): 279-91].

Adipose tissue lipolysis can communicate metabolic and nutritional status with other tissues to regulate whole body energy homeostasis. Fatty acids (FAs) released by adipocyte lipolysis can function as peroxisome proliferator-activated receptor (PPARα) ligands in the liver to upregulate a transcriptional program involved in increasing fatty acid oxidation (FAO) and very low density lipoprotein (VLDL) secretion. Furthermore, fatty acid ester of hydroxyl fatty acids (FAHFAs) released by adipocytes can act through a Gal-coupled receptor to suppress hepatic gluconeogenesis. These FAHFAs can also have autocrine effects by upregulating of de novo lipogenesis (DNL) and FA-esterification in adipocytes. FAs released by white adipocytes can activate GRP40 on β-cells in the pancreas to elicit insulin secretion which acts on brown adipose tissue (BAT) to promote greater FA uptake required for FAO. 12,13-diHOME, a lipolysis-derived lipokine secreted from brown adipocytes, can function in an autocrine manner to similarly promote further uptake and utilization of FAs. FAs released by white adipose tissue can be sensed by afferent nerves which feed back to the brain to increase sympathetic outflow to BAT through the sympathetic nervous system (SNS). Adipocytes can also secrete extracellular vesicles (EVs) in response to lipolysis that can further mediate tissue-tissue communication. [Yang, A. and Motillo, EP. Biochem. J. (2020) 477 (5): 985-1008]

Beta Adrenergic receptors. β-Adrenergic receptors (β-AR) and their associated guanine nucleotide regulatory protein (G protein)/adenylyl cyclase (AC) signal transduction pathways are central to the overall regulation of cardiac function. In particular, β-AR stimulation is a primary control point for modulation of heart rate and myocardial contractility. As of 2012, three subtypes of β-ARs had been identified at a molecular level, the β1, β2- and β3-AR [Wachter, S B and Gilbert, E M. Cardiology (2012) 122(2): 104-112, citing Frielle, T. et al., Proc. Natl Acad. Sci. USA (1987) 84: 720-4; Kobilka, B K et al. Proc. Natl Acad. Sci. USA (1987) 84: 46-50; Emorine, L J et al. Science (1989) 245: 118-21]. The β3-AR subtype has been associated primarily with metabolic regulation; however, there is some indication that unlike the β1- and β2-AR subtypes, it may inhibit myocardial contractility [Id., citing Gauthier, C. et al. J. Clin. Invest. (1996) 98: 556-62].

The nonfailing human heart expresses a mixed population of β-ARs, with approximately 80% of the expressed receptors being of the β1-AR subtype and approximately 20% of the β2-AR subtype [Id., citing Bristow, M R et al. Cir. Res. (1986) 59: 297-309; Bristow, M R et al. Circulation (1991) 84: 1024-39]. However, in the failing human heart, the β1-AR undergoes subtype selective downregulation at both the levels of mRNA and protein such that the β1-: β2-AR subtype proportions become approximately equal [Id., citing Bristow, M R et al. Circulation (1990) 82: 112-25]. Although there are some differences in the gene expression patterns based on the etiology of heart failure, overall, the extent of β1-AR downregulation correlates well with the severity of heart disease.

In cardiomyocytes, β-ARs (β1-, β2-ARs) are coupled to the stimulatory G protein, Gs, which in turn activates AC causing the production of cAMP from ATP. However, the coupling efficiency of the two receptor subtypes is markedly different. In homogenates of human ventricular myocardium, stimulation of the β2-AR is several fold more efficiently coupled to the production of cAMP than is the β1-AR [Id., citing Bristow, et al. Mol. Pharmacol. (1989) 35: 295-303; Levy, F O et al. Proc. Natl Acad. Sci. USA (1993) 90: 10798-802]. The second messenger, cAMP, activates protein kinase A (PK-A) which phosphorylates a number of protein targets including β-ARs, transcription factors, L-type calcium channels, phospholamban (PLB) and elements of the contractile apparatus.

In addition to activating PK-A, β-AR stimulation activates members of the G protein receptor protein kinase family, including βARK1 and βARK2. βARKs phosphorylate (β-ARs in an agonist-occupancy dependent manner. Phosphorylation reduces the affinity of the interaction of the receptor for the stimulatory G protein, Gs, and apparently increases the affinity of interaction for the inhibitory G protein, Gi [Id., citing Daaka, Y. et al. Nature (1997) 390: 88-91]. Phosphorylation also leads to the interaction of β-ARs with β-arrestin adaptor proteins which facilitate internalization of the receptors into clathrin-coated pits and endosomic vesicles.

Cardiomyocyte growth is modulated in part by β1-, β2- and α1-adrenergic receptors. Increases in signal transduction through these receptor pathways in the failing heart thus can contribute to pathological remodeling. Additionally, cardiac adrenergic receptor activation may promote dysrhythmias. [Wachter, S B and Gilbert, E M. Cardiology (2012) 122(2): 104-112].

Another consequence of increased exposure of cardiomyocytes to increased concentrations of norepinephrine is myocyte toxicity. In tissue culture systems there is both a time- and concentration-dependent relationship between norepinephrine exposure and cardiomyocyte death [Id., citing Mann, D L et al. Circulation (1992) 85: 790-804]. The toxic effects of norepinephrine incubation can be partially blocked with the addition of a β-adrenergic receptor antagonist and completely prevented with the addition of both a β- and α-blocker to the culture media. Apoptosis of cardiomyocytes can be induced in tissue culture by norepinephrine.

The term “administer” and its other grammatical forms as used herein means to give or to apply. The term “administering” as used herein includes in vivo administration, as well as administration directly to a tissue ex vivo or to a cell population in vitro.

The term “ADRB2” (or “adrenoceptor Beta 2”) as used herein refers to a protein coding gene encoding the beta-2-adrenergic receptor (beta2-AR), a G protein coupled adrenergic receptor.

The term “Annexin V” s used herein refers to a Ca2+ dependent phospholipid-binding protein with high affinity for phosphatidylserine (PS). It is a sensitive probe for PS exposure upon the cell membrane. In the early stages of apoptosis, translocation of phosphatidylserine (PS) occurs from the inner side of the plasma membrane to the outer layer, by which PS becomes exposed at the external surface of the cell. Translocation of PS to the external cell surface is not unique to apoptosis but occurs also during cell necrosis. The difference between these two forms of cell death is that during the initial stages of apoptosis the cell membrane remains intact, while at the very moment that necrosis occurs the cell membrane loses its integrity and becomes leaky. [Vermes, I. t al. J. Immunol. Methods (1995) 184 (1): 39-51].

The term “angiogenesis” as used herein refers to the sprouting and remodeling of small new capillaries from the preexisting blood vasculature.

The term “anti-inflammatory” as used herein refers to stopping or reducing inflammation. Major anti-inflammatory cytokines include interleukin (IL)-1 receptor antagonist, IL-4, IL-10, IL-11, and IL-13.

The terms “apoptosis” or “programmed cell death” refer to a highly regulated and active process that contributes to biologic homeostasis comprising a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways.

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to death domain (DD) containing receptors like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits inhibitor of apoptosis (IAP) proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome c is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and 7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation, can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. [Loberg, R D, et al., J. Biol. Chem. (2002) 277 (44): 41667-673]. It has been demonstrated to induce caspase 3 activation and to activate the proapoptotic tumor suppressor gene β53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “apoptotic bodies” as used herein refers to a form of extracellular vesicle released by dying cells during the process of apoptosis. They are quite variable in size and content and may contain a wide variety of cellular components, including micronuclei, chromatin remnants, cytosol portions, degraded proteins, DNA fragments or even intact organelles as a result of cell disassembly. [Battistelli, M. and Falcieri, E. Biology (Basel) (2020) 9 (1): 21].

The term “B-lymphocyte” or “B-cell” as used herein refers to one of the two types of antigen-specific lymphocytes responsible for adaptive immune responses, the other being the T cells. The function of B cells is to produce antibodies. B cells are divided into two classes. Conventional B cells have highly diverse antigen receptors (B-cell receptor, or BCR) and are generated in the bone marrow throughout life, emerging to populate the blood and lymphoid tissues. B-1 cells have much less diverse antigen receptors and form a population of self-renewing B cells in the peritoneal and pleural cavities.

The term “biological particle” as used herein refers to a minute portion, piece, fragment or amount (particle) derived from an organism. Biological particles include, without limitation, exosomes, extracellular vesicles, viral particles, bacterial particles, or other secreted particles comprising surface membranes.

The term “biomarker” (or “biosignature”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (e.g., choices of drug treatment or administration regimes). In evaluating potential therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community. A “predictive biomarker” is a biomolecule that indicates therapeutic efficacy, i.e., an interaction that exists between the biomolecule and therapy that impacts patient outcome. A “prognostic biomarker” is a biomolecule that indicates patient survival independent of the treatment received.

The term “cardiac fibroblast” or “CF” as used herein refers to a mesenchymal cell that resides in the cardiac interstitium [Chen, W. et al. Front. Bioeng. Biotechnol. (2021): 9: 599928, citing Souders, C A et al. Cir. Res. (2009) 105: 11164-76]. Cardiac fibroblasts are the primary drivers of cardiac remodeling in response to both physiological and pathological conditions [Id., citing Sadoshima, J. and Weiss, IN. J. Mol. Cell Cardiol. (2014) 70: 1]. In the immediate aftermath of myocardial infarction, they produce the scar tissue required to maintain the structural integrity of the chamber walls and prevent rupture, but the scar also impedes contractile performance, disrupts electromechanical coupling (which can generate arrhythmias), and induces mechanical stress that can lead to additional cardiomyocyte toxicity and infarct expansion.

The healing process after myocardial infarction can be divided into three distinct but overlapping stages-inflammation, proliferation, and maturity—and each stage is associated with a different CF phenotype [Id., citing Frangogiannis, NG, Antioxid. Redox Signal. (2006) 8: 1907-39].

Inflammation stage. The inflammatory phenotype is characterized by the secretion of cytokines and chemotactic factors that promote the infiltration of neutrophils and monocytes [Id., citing Sandanger, O. et al. Cardiovasc. Res. (2013) 99: 164-74; Shinde, A V and Frangogiannis, NG J. Mol. Cell Cardiol. (2014) 70: 74-82], which clear cellular debris, and by the production of matrix metalloproteinases (MMPs), which initiate remodeling by degrading the existing extracellular matrix (ECM).

Proliferation stage. In the proliferative stage, CFs transform into myofibroblasts, which express the contractile protein α-smooth muscle actin (α-SMA), vigorously proliferate, and become the dominant effector molecules of the repair process by secreting both anti-inflammatory and pro-angiogenic molecules, and by generating the new ECM [Id., citing Shinde, A V and Frangogiannis, NG J. Mol. Cell Cardiol. (2014) 70: 74-82; Ma, Y. et al., Trends Pharmacol. Sci. (2017) 38: 448-58]. Unlike fibroblasts, myofibroblasts are specialized cells that possess a more contractile and synthetic phenotype than fibroblasts. The lineage tracing of Periostin+ myofibroblasts did not show expression of the endothelial cell marker CD31 after MI [Id., citing (Kanisicak, O. et al., Nat. Commun. (2016) 7: 12260), suggesting the limited plasticity of myofibroblasts to transdifferentiate to other cardiac cells.

Maturation stage. CFs continue to produce anti-inflammatory cytokines (e.g., interleukin 10) and pro-fibrotic factors (e.g., transforming growth factor β1) during the early maturation stage [Id., citing Chen, W. and Frangogiannis, NG, Biochhim. Biophys. Acta (2013) 1833: 945-53], and then the number of myofibroblasts declines as the cells transition to a phenotype that promotes scar maturation and maintains homeostasis in the remodeled myocardium [Ma, Y. et al., Trends Pharmacol. Sci. (2017) 38: 448-58]. Fu et al., J. Clin. Invest. (2018) 128: 2127-43 dissected the dynamic states of CFs during post-myocardial infarction remodeling by lineage tracing of cells expressing Tcf21, Postn and Acta2 genes. Consistently, they identified proliferating activated fibroblasts early at post-MI day 2-4 and the α-SMA+ myofibroblasts at day 3-7 after injury. They also discovered a new differentiated state of fibroblasts in the mature scar beyond 10 days after injury. These CFs, termed as matrifibrocytes, highly express bone and cartilage-related ECM genes like Chad, Cilp2 and Comp, which are common gene signatures of chondrocytes and osteoblasts, making CFs more specialized to support mature scar. Importantly, deletion of these cells in the scar impaired the cardiac function, suggesting an indispensable role of matrifibrocytes in the homeostasis of scarred hearts.

The term “cardiac interstitium” as used herein refers to a system of diverse extracellular matrix (ECM) components organized into a complex, three-dimensional network that surrounds the cellular components of the heart [Borg, T K et al. (Borg and Caulfield 1981, Weber et al. 1994, Comper 1995). It contains noncollagen matrix proteins, proteoglycans, glycosaminoglycans, and a large reservoir of bioactive signaling molecules. [Eckhouse, S R and Spinale, FG. Heart Fail. Clin. (2012) 8 (1): 7-20, citing Baumgarten, G. et al. Circulation (2002) 105 (18): 2192-7; Booz, G W and Baker, KM. Cardiovasc. Res. (1995) 30 (4): 537-43; Briest, W. et al. Hypertension (2004) 44(4): 410-18; Chen, K. et al. Circ. Res. (2004) 95 (12): 1167-73; Dell'Italia, U et al. J. Clin. Invest. (1997) 100 (2): 253-8; Diwan, A. et al. Circulation (2004) 109 (2): 262-8; Ergul, A. et al. Am. J. Physiol. Heart Circ. Physiol. (2000) 278 (6): H2050-6; Filion, R J and Popel, A S, Am. J. Physiol. Heart Circ. Physiol. (2005) 288 (1): H263-79; Frangogiannis, N G et al. Circulation (2005) 111 (22): 2935-42; Fuchs, S. et al. Osteoarthritis Cartilage (2004) 12 (5): 409-18; Multani, M M et al. J. Thorac. Cardiovasc. Surg. (2005) 129 (3): 584-90; Rodriguez, Vita, J. et al. Cir. Res. (2005) 97 (2): 125-34; Romera, J R et al. J. Biol. Chem. (2005) 280 (15): 14378-84; Wei, C C et al. Circulation (1999) 99 (19): 2583-9]. The interstitium is highly organized and orchestrated whereby small disruptions in composition, spatial relationships, or content leads to altered myocardial systolic and/or diastolic performance.

The term “cardiac muscle” or “myocardium” as used herein refers to the thick middle layer of the heart. It is surrounded by a thin outer layer called the epicardium and an inner endocardium. The myocardium is a highly organized tissue, composed of several cell types that include smooth muscle cells, fibroblasts, and cardiac myocytes.

The term “cardiac remodeling” as used herein refers to a group of molecular, cellular and interstitial changes that manifest clinically as changes in size, mass, geometry and function of the heart. Talman, V. Kivela, R. Front. Cardiovasc. Med. (2018) 5: 101]. While physiological remodeling mainly includes cardiomyocyte hypertrophy, is reversible and improves cardiac function, pathological remodeling is accompanied with fibrosis and cardiomyocyte hypertrophy, atrophy, and apoptosis, and leads to deterioration of cardiac output. In physiological hypertrophy, the heart preserves its oxygen supply and balances the proportional increases in CM size and the extent of coronary microvasculature through angiogenesis. In heart failure, however, the pathological progression is associated with an imbalance between oxygen supply and demand, as CM hypertrophy is not matched by a corresponding increase in the vasculature.

The term “cardiokine” as used herein refers to proteins secreted by the heart.

The term “cardiomyocytes” or “cardiac myocyte” as used herein refers to the fundamental contractile cell of the myocardium. Cardiomyocytes are generally divided into pacemaker cells and force-producing ventricular and atrial cardiomyocytes. In order for the heart to function properly, additional cell types, such as blood and lymphatic endothelial cells (ECs), vascular smooth muscle cells (SMCs), fibroblasts, and pericytes are needed. In the myocardium, cardiomyocytes are physically connected and communicate with each other through gap junctions, adherens junctions, and desmosomes [Talman, V. and Kivela, R. Front. Cardiovasc. Med. (2018) 5: 101, citing Noorman, M. et al. J. Mol. Cell Cardiol. (2009) 47: 23-31]. They also communicate with other cell types in the heart through both direct physical contact and paracrine signaling. Cardiomyocytes produce and secrete proteins and peptides for paracrine signaling with other cardiac cells and for endocrine signaling with peripheral tissues. The main cardiokine regulating EC activation and proliferation is vascular endothelial growth factor (VEGF) that binds to and activates the VEGF receptor 2 (VEGFR2, also known as KDR and FLK1) in ECs [Id., citing Olsson, A K et al., Nat. Rev. Mol. Cell Biol. (2006) 7: 59-371]. VEGFR2 signaling induces angiogenesis, improving circulation, and thereby oxygen and nutrient availability in deprived areas to match the need of e.g., growing or infarcted heart. Additional extensively studied cardiokines inducing cardiac angiogenesis include the other VEGF family members VEGF-B [Id., citing Bry, M. et al. Physiol. Rev. (2014) 94: 779-84], VEGF-C [Id., citing Chen, H I et al. J. Clin. Invest. (2014) 124: 4899-14], and placental growth factor (PlGF) [Id., citing Accornero, F. and Molkentin, JD. Trends Cardiovasc. Med. (2011) 21: 220-4], as well as fibroblast growth factors (FGFs) [Id., citing Itoh, N. et al. Front. Cell Dev. Biol. (2016) 4: 110], hepatocyte growth factor (HGF) [Id., citing Gallo, S. et al. Clin. Sci. (2015) 129: 1173-93], and angiopoietin-1 [Id., citing Arita, Y. et al. Nat. Commun. (2014) 5: 4552]. Cardiomyocytes also produce and secrete several members of the TGF-β superfamily, which elicit both cardioprotective and detrimental effects on the heart. Of these, follistatin like-1 is one of the best characterized, and it has been shown to affect both CMs and ECs [Id., citing Shimano, M. et al. Circulation (2012) 126: e327-32].

The term “cardiomyopathy” as used herein refers to a myocardial disorder in which the heart muscle is structurally and functionally abnormal in the absence of coronary artery disease, hypertension, valvular disease, and congenital heart disease sufficient to explain the observed myocardial abnormality.

The term “cargo” as used herein refers to a load or that which is conveyed. With respect to exosomes and/or extracellular vesicles, the term cargo refers to a substance encapsulated in the exosome and/or extracellular vesicle. The compound or substance can be, e.g., a nucleic acid (e.g., nucleotides, DNA, RNA), a polypeptide, a lipid, a protein, or a metabolite, or any other substance that can be encapsulated in an exosome and/or extracellular vesicle.

The term “cargo profile” as used herein refers to the measurement of the abundance of cargo components (e.g., a nucleic acid (e.g., nucleotides, DNA, RNA), a polypeptide, a lipid, a protein, or a metabolite) that characterize the population of exosomes and/or extracellular vesicles.

The term “chemokine” as used herein refers to a class of chemotactic cytokines that orchestrate migration and positioning of immune cells within the tissues. Most chemokine receptors are transmembrane-spanning heterotrimeric G-protein-coupled receptors [Kohli, K. et al. Cancer Gene Therapy (2022) 29: 10-21]. Binding of a chemokine to its seven transmembrane G protein-coupled receptor triggers intracellular signaling that drives cell polarization, adhesion, and migration [Vilgelm, A E and Richmond, A. Front. Immunol. (2019) doi.org/10.3389/fimmu.2019.00333, citing Griffith, J W et al. Annu. Rev. Immunol. (2014) 32: 659-702; Nagarsheth, N. et al. Nat. Rev. Immunol. (2017) 17: 559-72]. Chemokines are divided into four families based upon structure: CXC, CC, CX3C, and C chemokines. Their receptors follow a similar nomenclature system, based upon the family of chemokines to which they bind. In addition, there is a family of atypical chemokine receptors that do not directly couple to G proteins but are reported to have a variety of roles in development, homeostasis, inflammatory disease, infection, and cancer [Id., citing Nibbs, R J, Graham, G J. Nat. Rev. Immunol. (2013) 13: 815-29]. Chemokines play an essential role in guiding the migration of both activating and suppressive immune cell types. The continuous migration of immune cells between lymphoid and nonlymphoid organs is a key feature of the immune system, facilitating the distribution of effector cells within nearly all compartments of the body. Reaching their correct position within primary, secondary, or tertiary lymphoid organs is a prerequisite to ensure unimpaired differentiation, maturation, and selection of immune cells, as well as their activation or functional silencing. The superfamilies of chemokines and chemokine receptors are of major importance in guiding immune cells to and within lymphoid and nonlymphoid tissues. [Schulz, O. et al. Annu. Rev. Immunol. (2016) 34: 203-42].

The term “CCAAT/enhancer binding protein” or “C/EBP/α, C/EBP/β and C/EBP/δ” refers to transcription factors of the basic-leucine zipper class. These proteins consist of three structural components which include a C-terminal leucine-zipper, a basic DNA-binding region and a N-terminal transactivating region. Dimerization through the leucine-zipper leads to formation of homo- and heterodimers which then bind with their two basic regions to other non-symmetric DNA-sequences in the promoter/enhancer regions of a variety of genes. Expression of C/EBP is prominent in adipocytes, hepatocytes and monocytes/macrophages, and here these proteins are involved in tissue-specific gene expression. Target genes for C/EBP include those for acute phase immune response genes in liver cells and for cytokine genes in monocytes/macrophages.

The term “CCR5” as used herein refers to the CC chemokine receptor 5 (CCR5), which is responsible for immune and inflammatory responses by mediation of chemotactic activity in leukocytes, although it is expressed on different cell types. It has been shown to act as the principal co-receptor for the human and simian immunodeficiency viruses (HIV-1, HIV-2, and SIV). [Zhang, H. et al. Nature Communic. (2021) 12: article 4151, citing Berger, E A et al. Annu. Rev. Immunol. (1999) 17: 657-700]. CCR5 exerts its physiological functions by binding to multiple chemokines, such as macrophage inflammatory protein (MIP-1a, also known as CCL3) and RANTES (regulated on activation normal T cell expressed and secreted, also known as CCL5) [Id., citing Bachelerie, F. et al. Pharmacol. Rev. (2014) 66: 1-79].

The term “cell sorting” as used herein refers to the physical separation of a population of cells or cell components into subpopulations. In positive selection for example, the target cells or components are directly labelled with, for example, antibodies conjugated to magnetic beads or fluorophores. The cells or components can subsequently be isolated by various techniques, including using a magnet (magnetic-activated cell sorting—MACS) or a flow cytometer suitable for fluorescence-activated cell sorting (FACS). Negative cell sorting involves labelling and removing unwanted cell types or cell components. For example, the unwanted cell types can be bound using magnetic beads coupled to antibodies or ligands that target specific cell surface proteins.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “derived from” as used herein encompasses any method for receiving, obtaining, or modifying something from a source of origin.

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like. “Assay markers” are measurable components whose presence or absence can be detected and correlated to a particular detectable response.

The term “detectable response” as used herein refers to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “dilated cardiomyopathy” or “DCM” as used herein refers to a condition in which one or both ventricles is enlarged (dilated), which causes the thick muscular wall to stretch, becoming thinner and weaker. This affects the heart's ability to pump enough oxygen-rich blood to the rest of the body.

The terms “disease” or “disorder” as used herein refer to an impairment of health or a condition of abnormal functioning.

The term “effector cell” as used herein refers to a cell that carries out a final response or function. The main effector cells of the immune system, for example, are activated lymphocytes and phagocytes.

The term “effector functions” as used herein refers to the actions taken by effector cells and antibodies to eliminate foreign entities, and includes, without limitation, cytokine secretion, cytotoxicity, and antibody-mediated clearance.

The term “encapsulated” as used herein refers to being enclosed in a capsule (meaning a membranous envelope enclosing a part).

The term “enrich” as used herein refers to increasing the proportion of a desired substance, for example, to increase the relative frequency of a subtype of cell or cell component compared to its natural frequency in a cell population. Positive selection, negative selection, or both are generally considered necessary to any enrichment scheme. Selection methods include, without limitation, magnetic separation and fluorescence-activated cell sorting (FACS).

The term “endoplasmic reticulum stress” or “ER stress” as used herein is a cellular condition resulting from disruptions in the protein folding pathway of the endoplasmic reticulum (ER) that prevent the proper turnover of misfolded or unfolded proteins, potentially leading, to their accumulation and aggregation. [Chadwick, S R and Lajoie, P Frong Cell Dev. Biol. (2019) 7:84]. It can be induced by environmental factors. The cell resolves maisfolded protein stress via two major stress response pathways: the heat shock response (H1SR) (Verghese et al., 2012), which handles misfolded proteins in the cytoplasm, and the unfolded protein response (UPR), which takes place in the ER [Id., citing Kohno, K, et al Molec. Cell Biol. (1993) 13: 877-0.90 Cox, J S and Walter, P. Cell (1996) 87:391-404 Liu, Y and Chang, A. EMBO J, (2008) 27: 1049-5]. In mammals, three distinct ER stress sensors exist inosizol requiring kinase 1 (IRE1) [Sidrauski. C. and Walter, P. Cell (1997) 90: 1031-9; shda, H. et al Cell (2001) 107: 881-1, Calfon, M. et al. Nature (2002) 41.2-96], dub]e-stranded RNA-activated protein kinase like endoplasmic reticulum kinase (PERK) [Harding, H P et al. Mol. Cell (2000) 6: 1099-1108], and activating transcription factor 6 (AT1F6) [Yoshida, H. et al. J. Biol. Chem. (1998) 273: 33741-9]. When the UPR is activated, ER chaperone proteins (such as BIP) dissociate from these sensors, allowing their activation which in turn activates downstream signaling pathways [Welibinda, A A et al. Gne Expr. (1999) 7:293-300; Shen, J. et al Dev. Cell (2002) 3: 99-11; Ma, Y and Hendershot, L M J. Chen. Neuroanat. (2004) 28: 51-65; Pincus, D. et al Plos Biol. (2010) 8: e1000415]. Effector proteins from each of the three pathways bind to UPR response element (UPRE) sequences ii gene promoters. A cell may activate over 400 UPR target genes involved in responding to ER stress, such is chaperone proteins, ribosome biogenesis genes, ERAD effectors, and genes to expand the ER lumen [Welibinda, A A et al. Gene Expr. (1999) 7: 293-300 Ma, Y and Hendershot, L1 J. Chem. Neuroanat. (2004) 28: 51-65: Aragón, T. et al Nature (2009) 457: 736-40]. Upregulation of such genes contributes to adapt the ER folding environment to the new misfolded protein burden.

The term “exosomes and/or extracellular vesicles” as used herein refers to extracellular bilayered, membrane-bound vesicles including exosomes, microvesicles, and apoptotic bodies (ApoBDs) released by all cells in different physiological and patho-physiological conditions. Extracellular vesicles (EVs) are characterized by differences in size, origin, and content and different types have different functions. EVs are also referred to as microparticles, microvesicles, microsomes, lipid vesicles, apoptotic blebs, or exosomes, depending on the basis of their biogenesis or release pathways. EVs are characterized by their size (40-100 nm for exosomes, 100-500 nmn diameter for the larger microvesicles, and 500 nmn-2 μm for apoptotic bodies), by their cells of origin, such as megaryocytes, platelets, red blood cells, and endothelial cells, and by their intravesicular contents. Their inner content depends on their cells of origin and can include proteins, sugars, lipids, adhesion integrins, growth factors, protease inhibitors, and different types of genetic material such as double stranded DNA, mRNA, or mnicroRfNA. [Battistelli M, Falcieri E. Apoptotic Bodies: Particular Extracellular Vesicles involved in Intercellular Communication. Biology (Basel). 2020 Jan. 20; 9(1):21. doi: 10.3390/biclogy901002. PMID: 31968627; PMCI ID: [MC7 168913].

Biogenesis. Under physiological conditions, exosomes and/or extracellular vesicles are generated in a process that involves double invagination of the plasma membrane and the formation of intracellular multivesicular bodies (MVBs) containing intraluminal vesicles (ILVs). ILVs are ultimately secreted as exosomes and/or extracellular vesicles with a size range of ˜40 to 160 nm in diameter through MVB fusion to the plasma membrane and exocytosis. The first invagination of the plasma membrane forms a cup-shaped structure that includes cell-surface proteins and soluble proteins associated with the extracellular milieu. This leads to the de novo formation of an early-sorting endosome (ESE) and in some cases may directly merge with a preexisting ESE. The trans-Golgi network and endoplasmic reticulum can also contribute to the formation and the content of the ESE (Kalluri, R., LeBleu, VS. Science (2020) 367 (6478): eaau6977, citing Kalluri, R. J. Clin. Invest. (2016) 126: 1208-15; van Neil, G. et al. Nat. Rev. Mol. Cell Biol. (2018) 19: 213-28; McAndrews, KM, Kalluri, R. Mol. Cancer (2019) 18: 52; Mathieu, M. et al. Nat. Cell Biol. (2019) 21: 9-17; Willms, E. et al. Front. Immunol. (2018) 9: 738; Hessvik, NP, Llorente, A. Cell Mol. Life Sci. (2018) 75: 193-208). ESEs can mature into late-sorting endosomes (LSEs) and eventually generate MVBs, which are also called multivesicular endosomes. MVBs form by inward invagination of the endosomal limiting membrane (that is, double invagination of the plasma membrane). This process results in MVBs containing several ILVs (future exosomes and/or extracellular vesicles). The MVB can either fuse with lysosomes or autophagosomes to be degraded or fuse with the plasma membrane to release the contained ILVs as exosomes and/or extracellular vesicles [Id., citing Kahler, C., Kalluri, R. J. Mol. Med. (2013) 91: 431037]. The biogenesis of microvesicles (100-500 nm size) occurs via the direct outward blebbing and pinching of the plasma membrane releasing the nascent microvesicle into the extracellular space [Tricarico, C.; Clancy, J.; D'Souza-Schorey, C. Biology and biogenesis of shed microvesicles. Small GTPases 2017, 8, 220-232]. During physiological and patho-physiological condition, dying cells, release 500 nm-2 m vesicular apoptotic bodies that can be more abundant than exosomes or MVs under specific conditions and appear variable in size, structure, and composition. Apoptosis progresses through several stages, first nuclear chromatin condensation, then nuclear splitting and the frequent appearance of micronuclei, then membrane blebbing and finally, splitting of the cellular content into distinct membrane-enclosed vesicles, termed apoptotic bodies. [Battistelli M, Falcieri E. Apoptotic Bodies: Particular Extracellular Vesicles Involved in Intercellular Communication. Biology (Basel). 2020 Jan. 20; 9(1):21. doi: 10.3390/biology9010021. PMID: 31968627; PMCID: PMC7168913].

Heterogeneitv: The heterogeneity of extracellular vesicles is thought to be reflective of their size, content, functional impact on recipient cells, and cellular origin. During their secretion, they acquire surface proteins and lipids from their cell of origin. They naturally transport cytoplasmic and nuclear contents such as lipids, proteins, mRNA, miRNA, and genomic DNA between cells.

Biomarkers. There is general agreement that the membranes of extracellular vesicles are specifically enriched in tetraspanins CD9, CD37, CD63, CD81, and CD82.

Role. Extracellular vesicles are mediators of near and long-distance intercellular communication in health and disease and affect various aspects of cell biology.

The term “expression” and its various grammatical forms as used herein refers to the action of a gene in the production of mRNA or a protein or phenotype. “Level of expression” refers to the degree to which a particular gene produces its effect(s) in an organism in the form of specific mRNA or protein.

The term “extract” and its other grammatical forms as used herein refers to a technique that involves separating a desired substance from a mixture. In a liquid-liquid (solvent) extraction, separation is based on the solubility of compounds in solvent. In a liquid-solid extraction, the extraction process involves the extraction or transfer of a compound from a solid using a solvent

The term “extracellular vesicles (EVs)” as used herein refers to nano/micro sized, membrane-bound vesicles released from cells that can transport cargo—including lipids, DNA, RNA, and proteins between cells as a form of intercellular communication. Different EV types, including microvesicles (MVs) exosomes, oncosomes, and apoptotic bodies, have been characterized on the basis of their biogenesis or release pathways. Microvesicles bud directly from the plasma membrane, are 100 nanometers (nm) to 1 micrometer (μm) in size, and contain cytoplasmic cargo (Zaborowki, M P et at BioScience (2015) 65 8): 783-9 citing Heijnen, H F et al Blood (1999) 94: 3791-99). Another EV subtype, exosomes by the fusion between multivesicular bodies and the plasma membrane, by which multivesicular bodies release smaller vesicles (exosomes) whose diameters range from 4) to 120 nm (Id., citing E1 Antaloussi, S. et al. Nature Reviews Drug Discovery (2013) 12: 347-57; Coc cci. E and Meldolesi J. Trends in Cell Biology (2015) 25: 364-72). Dying cells, release vesicular apoptotic bodies (50 nm-2 μm) that can be more abundant than exosomes and or extracellular vesicles or MVs under specific conditions and can vary in content between biofluids (Id., citing Thery, C. et al. J. Immunology (2001) 1666: 7309-18; E1 Andaloussi, S et al, Nature Reviews Drug Discovery (2013) 12: 347-57). Membrane protrusions can also give rise to large EVs, termed oncosomes (1-10 μm), which are produced primarily by malignant cells in contrast to their nontransformed counterparts (Id., citing Di Vizio, D. et al. Am. J. Pathol (2012) 181: 1573-84; Morello, M. et al. Cell Cycle (2013) 12: 3526-36).

The term “fatty acid binding protein 4” or FABP4, also known as aP2, is a cytoplasmic fatty acid chaperone expressed primarily in adipocytes and myeloid cells, which couples intracellular lipids to biological targets and signaling pathways. FABP4 is induced by peroxisome proliferator-activated receptor γ (PPARγ), a master regulator of adipogenesis and insulin responsiveness, but exerts metabolic and immunologic activities opposite to those of PPARγ. FABP4 negatively regulates PPARγ levels in macrophages and adipocytes, thereby attenuating adipocyte differentiation. [Garin-Shkolnick, T. et al. Diabetes (2014) 63 (3): 900-911].

The term “gene” as used herein refers to a locatable segment of a genomic sequence corresponding to a unit of inheritance, which is associated with regulatory regions, transcribed regions that code for a protein or RNA product, and other functional sequence regions.

The term “Nd1” as used herein refers to the protein coding gene for mitochondrially encoded NADH dehydrogenase 1. It enables NADH dehydrogenase (ubiquinone) activity and is involved in mitochondrial electron transport, NADH to ubiquinone and mitochondrial respiratory chain complex I assembly. NADH dehydrogenase (ubiquinone), which is located in the mitochondrial membrane, is part of the mitochondrial respiratory chain complex.

The term “SDHC gene” as used herein refers to the gene that provides instructions for making one of four subunits of the succinate dehydrogenase (SDH) enzyme.

The term “Cytb” as used herein refers to the gene that provides instructions for making the protein called cytochrome b.

The term “MT-CO1” (or “Mitochondrially Encoded Cytochrome C Oxidase I”) refers to a protein coding gene. It contributes to cytochrome-c oxidase activity and is involved in mitochondrial electron transport, cytochrome c to oxygen.

The term “COX5A” refers to a gene encoding mitochondrial cytochrome c oxidase subunit 5A, a protein coding gene. It encodes a component of cytochrome c oxidase, the last enzyme in the mitochondrial electron transport chain which drives oxidative phosphorylation.

The term “MT-ATP6” refers to a gene provides information for making mitochondrially encoded ATP synthase 6. This enzyme (complex V) is responsible for the final step of oxidative phosphorylation.

The term “SLC25A4” as used herein refers to a gene that provides the instructions for making a protein called adenine nucleotide translocase type 1 (ANT1). ANT1 functions in mitochondria.

The term “peroxisome proliferative activated receptor, gamma, coactivator 1 alpha” or “PPARGC1A” or PPARG Coactivator 1 Alpha” or “PGC-1α” as used herein refers to a protein coding gene that encodes a transcriptional coactivator that induces and coordinates gene expression regulating mitochondrial biogenesis, respiration, hepatic gluconeogenesis, thermogenic program in brown fat and muscle fiber-type switching.

As used herein, the term “gene expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” Expression may also refer to the post-translational modification of a polypeptide or protein.

The term “healthy subject” as used herein refers to a subject having no signs or symptoms of a disease.

The term “HFF-1” cells refers to an immortalized human foreskin fibroblast cell line.

The term “immune evasion” as used herein refers to mechanisms used by pathogens to avoid detection and/or elimination by host immune defenses.

The term “immune homeostasis” refers to the delicate and finely regulated balance of appropriate immune activation and suppression in tissues and organs, driven by a myriad of cellular players and chemical factors. [da Gama Duarte, J. et al. Immunology and Cell Biology (2018) 96: 497-506]

The terms “immune response”, “immunological response” and “immune-mediated response” are used interchangeably herein to refer to any functional expression of a subject's immune system, against either foreign or self-antigens, whether the consequences of these reactions are beneficial or harmful to the subject.

The human immune system is a complex arrangement of cells and molecules that maintain immune homeostasis to preserve the integrity of the organism by discriminating self-from non-self and elimination of all elements judged to be dangerous. Responses in the immune system may generally be divided into two arms, referred to as “innate immunity” and “adaptive immunity.” The two arms of immunity do not operate independently of each other but rather work together to elicit effective immune responses.

Innate immunity is a nonspecific fast response to pathogens that is predominantly responsible for an initial inflammatory response via a number of soluble factors, including the complement system and the chemokine/cytokine system, and a number of specialized cell types, including mast cells, macrophages, dendritic cells (DCs), and natural killer cells (NKs). This response is generally driven by germline encoded pattern recognition proteins. The successful activation of innate immunity informs the development of the subsequent adaptive immune response.

Adaptive immunity, by contrast, involves a specific, delayed and longer-lasting response by various types of cells that create long-term immunological memory against a specific antigen. It can be further subdivided into cellular and humoral branches, the former largely mediated by T cells and the latter by B cells. This response is generally driven by proteins resulting from germline rearrangements of DNA sequences in T cells (e.g., T Cell Receptor, TCR) and B cells (e.g., B Cell Receptor, BCR) amplified by somatic hypermutation to enable the generation of innumerable recognition proteins (TCRs and antibodies) specific for the wide array of molecules an individual is likely to encounter in a lifetime. Adaptive immunity further encompasses cell lineage members of the adaptive arm that have effector functions in the innate arm (e.g., NK-T cells), thereby bridging the gap between the innate and adaptive immune response.

Generally speaking, immune responses are initiated by an encounter between an individual and a foreign substance (e.g., an infectious microorganism or a mutated cell). The infected individual rapidly responds with both a humoral immune response with the production of antibody molecules specific for the antigenic determinants/epitopes of the immunogen, and a cell mediated immune response with the expansion and differentiation of antigen-specific regulatory and effector T-lymphocytes, including cells that produce cytokines and killer T cells, capable of lysing infected cells. Primary immunization with a given microorganism evokes antibodies and T cells that are specific for the antigenic determinants/epitopes found on that microorganism; these usually fail to recognize or recognize only poorly antigenic determinants expressed by unrelated microbes [Paul, W. E., “Chapter 1: The immune system: an introduction,” Fundamental Immunology, 4th Edition, Ed. Paul, W. E., Lippincott-Raven Publishers, Philadelphia, (1999), at p. 102].

As a consequence of this initial response, the immunized individual develops a state of immunologic memory. If the same or a closely related microorganism is encountered again, a secondary response ensues. This secondary response generally consists of an antibody response that is more rapid, greater in magnitude and composed of antibodies that bind to the antigen with greater affinity and that are more effective in clearing the microbe from the body, and a similarly enhanced and often more effective T-cell response. However, immune responses against infectious agents do not always lead to elimination of the pathogen [Paul, W. E., “Chapter 1: The immune system: an introduction,” Fundamental Immunology, 4th Edition, Ed. Paul, W. E., Lippincott-Raven Publishers, Philadelphia, (1999), at p. 102].

The term “immune system” as used herein refers to the body's defenses against disease, which comprise the innate immune system and the adaptive immune system. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

The terms “immunomodulatory” and “immune modulatory” are used interchangeably herein to refer to a substance, agent, or cell that is capable of augmenting or diminishing immune responses directly or indirectly, e.g., by expressing chemokines, cytokines and other mediators of immune responses.

The term “infection” as used herein refers to the invasion and multiplication of microorganisms, such as bacteria, viruses, fungi, and parasites that are not normally present within the body.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “inflammatory mediators” as used herein refers to the molecular mediators of the inflammatory process. These soluble, diffusible molecules act locally at the site of tissue damage and infection and at more distant sites. Some inflammatory mediators are activated by the inflammatory process, while others are synthesized and/or released from cellular sources in response to acute inflammation or by other soluble inflammatory mediators. Inflammatory mediators can be divided into seven groups based on their biochemical properties: vasoactive amines, vasoactive peptides, cytokines, chemokines, fragments of complement components, lipid mediators, and proteolytic enzymes. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IFN-γ), and interleukin-12 (IL-12).

Among the pro-inflammatory mediators, IL-1, IL-6, and TNF-α are known to activate hepatocytes in an acute phase response to synthesize acute-phase proteins that activate complement. Complement proteins can be activated either directly by pathogens or indirectly through pathogen-bound antibodies. This activation initiates a cascade of reactions occurring on the surface of pathogens, resulting in the generation of active components with diverse effector functions. IL-1, IL-6, and TNF-α also activate bone marrow endothelium to mobilize neutrophils, and function as endogenous pyrogens, raising body temperature, which helps eliminating infections from the body. A major effect of cytokines is to act on the hypothalamus, altering the body's temperature regulation, and on muscle and fat cells, stimulating the catabolism of the muscle and fat cells to elevate body temperature. At elevated temperatures, bacterial and viral replication are decreased, while the adaptive immune system operates more efficiently.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by a range of 5% to 99%, inclusive, i.e., at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “innate immune response” as used herein refers to the various mechanisms encountered by a pathogen or transformed cell before adaptive immunity is induced, such as anatomical barriers, antimicrobial peptides, the complement system, and macrophages and neutrophils carrying nonspecific pattern-recognition receptors. Innate immunity is present in all individuals at all times, does not increase with repeated exposure, and discriminates between groups of similar pathogens, rather than responding to a particular pathogen.

The term “innate lymphoid cells” or “ILCs” as used herein refers to a class of innate immune cells having overlapping characteristics with T cells but lacking an antigen receptor. They arise in several groups, ILC1, ILC2, ILC3, and NK cells, which exhibit properties roughly similar to TH1, TH2, TH17 and CD8 T cells, respectively.

The term “interleukin” as used herein refers to a cytokine secreted by white blood cells as a means of communication with other white blood ceils. For example, interleukin-8 (or “IL-8”) is produced by phagocytes and mesenchymal cells exposed to inflammatory stimuli (e.g., interleukin-1 or tumor necrosis factor) and activates neutrophils inducing chemotaxis, exocytosis and the respiratory burst. In vivo, L-8 elicits a massive neutrophil accumulation at the site of infection.

The term “interleukin-6” or “IL-6” as used herein refers to a cytokine produced by activated macrophages and inflammatory adipocytes which has many effects, including lymphocyte activation, the stimulation of antibody production and the induction of fever.

The term “innate lymphoid cells” or “ILCs” refers to a class of innate immune cells having overlapping characteristics with T cells but lacking an antigen receptors. They arise in several groups, namely ILC1, ILC2 and ILC3.

The term “isolated” is used herein to refer to material, such as, but not limited to, an EV, an adipome, a nucleic acid, a peptide, a polypeptide, or a protein, which is: (1) substantially or essentially free from components that normally accompany or interact with it as found in its naturally occurring environment. The terms “substantially free” or “essentially free” are used herein to refer to more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “lipogenesis” as used herein refers to the synthesis of fatty acids from nonlipid or lipid precursors (e.g., acetyl CoA, malonyl CoA). It encompasses the processes of fatty acid synthesis and subsequent triglyceride synthesis and takes place in both liver and adipose tissue. [Kersten, S. EMBO Rep. (2001) 2(4): 282-6].

The term “lipolysis” as used herein refers to the metabolic process through which fats and other lipids are broken down via hydrolysis to release fatty acids.

The term “lymphocyte” refers to a small white blood cell formed in lymphatic tissue throughout the body and in normal adults making up about 22-28% of the total number of leukocytes in the circulating blood that plays a large role in defending the body against disease.

Individual lymphocytes are specialized in that they are committed to responding to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. Lymphocytes are much more common in the lymphatic system, and include B cells, T cells, natural killer T (NKT) cells, and natural killer (NK) cells. There are two broad categories of lymphocytes, namely T cells and B cells.

The term “lymphocyte activation” or “activation” refers to stimulation of lymphocytes by specific antigens, nonspecific mitogens, or allogeneic cells resulting in synthesis of RNA, protein and DNA and production of lymphokines, the soluble product of lymphocytes; it is followed by proliferation and differentiation of various effector and memory cells.

The term “macrophage” as used herein refers to a mononuclear, actively phagocytic cell arising from monocytic stem cells in the bone marrow. These cells are widely distributed in the body and vary in morphology and motility. Phagocytic activity is typically mediated by serum recognition factors, including certain immunoglobulins and components of the complement system, but also may be nonspecific. Macrophages also are involved in both the production of antibodies and in cell-mediated immune responses, particularly in presenting antigens to lymphocytes. They secrete a variety of immunoregulatory molecules. Polarization of immune effector cells is exemplified by the induced differentiation of macrophages into M1 and M2 macrophages during type 1 and type 2 immune responses, respectively [Ma, Q., Frontiers in Immunol. (2020) 11: 1060, citing Biswas, S K and Mantovani, A. Nat. Immunol. (2010) 11: 889-96; Murray, P J and Wynn, TA. Nat. Rev. Immunol. (2011) 11: 723-37; Hussell, T. and Bell, TJ. Nat. Rev. Immunol. (2014) 14: 81-93; Murray, P J et al. Immunity (2014) 41: 14-20]. M2 cells can be further separated into distinctive 2a, 2b, 2c, and 2d subgroups according to their activating signals, secreted cytokines, and activities. IL-4 and IL-13 are major inducers of M2a polarization. M2a cells regulate tissue repair and the internalization of proinflammatory molecules by upregulating the expression of arginase-1 (ARG1), mannose receptor C-type 1 (MRC1, CD206), Major Histocompatibility Complex class II (MHC class II), IL-10 and TGF-β. M2b cells are activated by immune complexes or lipopolysaccharides (LPS) and produce IL-1, IL-6, IL-10, and TNF-α to activate Th2 cells and anti-inflammatory activities. M2c cells are activated in response to IL-10, TGF-β and glucocorticoids; M2cs produce IL-10 and TGF-β to suppress inflammatory responses. Polarization of macrophages exhibits considerable plasticity with regard to their cell source, inducing signal, mechanism of differentiation, and interconversion between subtypes [Hobson-Gutierrez, SA and Carmona-Fontaine, C. Dis. Model Mech. (2018) 11: dmm034462.10.1242/dmm.034462., citing Biswas, SK Biswas, S K and Mantovani, A. Nat. Immunol. (2010) 11: 889-96; Murray, P J and Wynn, TA. Nat. Rev. Immunol. (2011) 11: 723-37; Hussell, T. and Bell, TJ. Nat. Rev. Immunol. (2014) 14: 81-93; Murray, P J et al. Immunity (2014) 41: 14-20]

As used herein, the terms “marker” or “cell surface marker” are used interchangeably to refer to an antigenic determinant or epitope found on the surface of a specific type of cell. Cell surface markers can facilitate the characterization of a cell type, its identification, and eventually its isolation. Cell sorting techniques are based on cellular biomarkers where a cell surface marker(s) may be used for either positive selection or negative selection, i.e., for inclusion or exclusion, from a cell population.

The term “modulate” as used herein means to regulate, alter, adapt, or adjust to a certain measure or proportion.

The term “multiplicity of infection” or “MOI” as used herein refers to the ratio of the number of infectious agents to cells in culture. For example, the MOI of T. cruzi parasites to cells disclosed herein is 3:1.

The term “Myd88” as used herein refers to an adaptor protein that functions in signaling by all TLR proteins except TLR3.

The term “mitochondrial oxidative phosphorylation (OXPHOS) system” as used herein refers to the final biochemical pathway in the production of ATP. It consists of five multiprotein complexes (I-V), the individual subunits of which are encoded either by the mitochondrial or by the nuclear genome. These complexes are known as NADH: ubiquinone oxidoreductase (complex I), succinate dehydrogenase (complex II), ubiquinol-cytochrome c oxidoreductase (complex III, or cytochrome bc1 complex), cytochrome c oxidase (complex IV), and ATP synthase (complex V). [Sharma, L K et al. Curr. Med. Chem. (2009) 16 (10): 1266-77].

The term “necrosis” as used herein refers to an irreversible insult that interferes with a vital structure or function of an organelle (plasma membrane, mitochondria, etc.) of a cell and does not trigger apoptosis. Such insults include infectious agents (e.g., bacteria, viruses, fungi, parasites), oxygen deprivation or hypoxia, and extreme environmental conditions such as heat, radiation, or exposure to ultraviolet irradiation. At the cellular level, necrosis is characterized by cell and organelle swelling, ATP depletion, increased plasma membrane permeability, release of macromolecules and eventually inflammation. The processes by which cells undergo death by necrosis vary according to the cause, organ and cell type. While the best studied is ischemic necrosis of cardiac myocyte, the basic processes involved are comparable to those in other organs. Some of the unfolding events may occur simultaneously; others may be sequential. These are:
(1) interruption of blood supply decreases delivery of oxygen and glucose;
(2) anaerobic glycolysis leads to overproduction of lactate and decreased intracellular pH;
(3) distortion of the activities of ionic pumps in the plasma membrane skews the ionic balance of the cell;
(4) activation of phospholipase A₂ (PLA₂) and proteases disrupts the plasma membrane and cytoskeleton;
(5) the lack of oxygen impairs mitochondrial electron transport, thus decreasing ATP synthesis and facilitating production of ROS;
(6) mitochondrial damage promotes the release of cytochrome c to the cytosol;
(7) the cell dies. [Rubin's Pathology: Clinicopathologic Foundations of Medicine, 5^{th} Ed. McDonald, J M, Michalopoulos, G K, Trojanowski, J Q, Ward, P A, Eds Lippincott Williams & Wilkins, M D (2008), pp. 23-26.]

types of necrosis include, for example:

liquefactive necrosis (occurs when the rate of dissolution of the necrotic cell is considerably faster than the rate of repair; the polymorphonuclear leukocytes of the acute inflammatory reaction contain potent hydrolases capable of digesting dead cells. [Rubin's Pathology: Clinicopathologic Foundations of Medicine, 5^{th} Ed. McDonald, J M, Michalopoulos, G K, Trojanowski, J Q, Ward, P A, Eds Lippincott Williams & Wilkins, M D (2008), pp. 23-26.]

coagulative necrosis, which refers to light microscopic alterations in a dead or dying cell including pyknosis (the nucleus becomes smaller and stains deeply basophilic as chromatin clumping continues); karyorrhexis (meaning the pyknotic nucleus breaks up into many smaller fragments scattered about the cytoplasm); and karyolysis (referring to the extrusion of the pyknotic nucleus from the cell or progressive loss of chromatin staining). [Rubin's Pathology: Clinicopathologic Foundations of Medicine, 5^{th} Ed. McDonald, J M, Michalopoulos, G K, Trojanowski, J Q, Ward, P A, Eds Lippincott Williams & Wilkins, M D (2008), pp. 23-26.]

caseous necrosis (which is characteristic of tuberculosis and attributed to the toxic effects of the microbacterial cell wall. In caseous necrosis, the necrotic cells fail to retain their cellular outlines; the dead cells persist indefinitely as amorphous, coarsely granular, eosinophilic debris). [Rubin's Pathology: Clinicopathologic Foundations of Medicine, 5^{th} Ed. McDonald, J M, Michalopoulos, G K, Trojanowski, J Q, Ward, P A, Eds Lippincott Williams & Wilkins, M D (2008), pp. 23-26.]

fat necrosis, which specifically affects adipose tissue and most commonly results from pancreatitis or trauma; the process begins when digestive enzymes normally found only in the pancreatic duct and small intestine, are released from injured pancreatic acinar cells and ducts into the extracellular spaces. On extracellular activation, these enzymes digest the pancreas itself and surrounding tissues, including adipose cells. [Rubin's Pathology: Clinicopathologic Foundations of Medicine, 5^{th} Ed. McDonald, J M, Michalopoulos, G K, Trojanowski, J Q, Ward, P A, Eds Lippincott Williams & Wilkins, M D (2008), pp. 23-26.]

fibrinoid necrosis (which is an alteration of injured blood vessels in which insudation and accumulation of plasma proteins cause the wall to stain intensely with eosin. The eosinophilia of the accumulated plasma proteins obscures the underlying alterations in the blood vessel making it difficult, if not impossible, to determine whether there truly is necrosis in the vascular wall.) [Rubin's Pathology: Clinicopathologic Foundations of Medicine, 5^{th} Ed. McDonald, J M, Michalopoulos, G K, Trojanowski, J Q, Ward, P A, Eds Lippincott Williams & Wilkins, M D (2008), pp. 23-26.]

gangrenous necrosis (used to describe ischemic necrosis of the lower limbs, sometimes upper limbs or digits). [Rubin's Pathology: Clinicopathologic Foundations of Medicine, 5^{th} Ed. McDonald, J M, Michalopoulos, G K, Trojanowski, J Q, Ward, P A, Eds Lippincott Williams & Wilkins, M D (2008), pp. 23-26.]

The term “neutrophils” or “polymorphonuclear neutrophils (PMNs)” as used herein refers to the most abundant type of white blood cells in mammals, which form an essential part of the innate immune system. They form part of the polymorphonuclear cell family (PMNs) together with basophils and eosinophils. Neutrophils are normally found in the blood stream. During the beginning (acute) phase of inflammation, particularly as a result of bacterial infection and some cancers, neutrophils are one of the first-responders of inflammatory cells to migrate toward the site of inflammation. They migrate through the blood vessels, then through interstitial tissue, following chemical signals such as interleukin-8 (IL-8) and C5a in a process called chemotaxis, the directed motion of a motile cell or part along a chemical concentration gradient toward environmental conditions it deems attractive and/or away from surroundings it finds repellent.

The term “NFκB” as used herein refers to a proinflammatory transcription factor. It switches on multiple inflammatory genes, including cytokines, chemokines, proteases, and inhibitors of apotosis, resulting in amplification of the inflammatory response [Barnes, P J, (2016) Pharmacol. Rev. 68: 788-815] The molecular pathway involved in NF-κB activation include several kinases. The classic (canonical) pathway for inflamatory stimuli and infections to activate NF-κB signaling involve the IKK (inhibitor of κB kinase) complex, which is composed of two catalytic subunits, IKK-α and IKK-β, and a regulatory subunit IKK-γ(or NFκB essential modulator [Id., citing Hayden, M S and Ghosh, S (2012) Genes Dev. 26: 203-234]. The IKK complex phosphorylates Nf-κI-bound IκBs, targeting them for degradation by the proteasome and thereby releasing NF-κB dimers that are composed of p65 and p50 subunits, which translocate to the nucleus where they bind to KB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-α homodimers and releases RelB and processes p100 to p52 in response to certain members of the TNF family, such as lymphotoxin-β Id, citing Sun, SC. (2012) Immunol. Rev. 246: 1254-14]. This pathway switches on different gene sets and may mediate different immune functions from the canonical pathway. Dominant—negative IKK-β inhibits most of the proinflammatory functions of NF-κB, whereas inhibiting IKK-α, has a role only in response to limited stimuli and in certain cells such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (20 Int. Arch. Allergy Immunol. 151: 179-89]

The term “oxidative stress” as used herein refers to a condition where the levels of reactive oxygen species (“ROS”) significantly overwhelm the capacity of antioxidant defenses, leading to potential damage in a biological system. An oxidative stress condition can be caused by either increased ROS formation or decreased activity of antioxidants or both. Not all increases in ROS levels in a biological system are associated with injury. Under certain circumstances, small transient increases in ROS levels can be employed as a signaling mechanism, leading to physiological cellular responses. [Li, R. et al. React. Oxyg. Species (Apex) (2016) I (1): 9-21].

The term “PAMPs” is an abbreviation for pathogen-associated molecular patterns. PAMPS are structural patterns present in components or products common to a wide variety of microbes but not host cells. PAMPS are ligands for pattern recognition molecules (PRMs).

The term “parasite” as used herein refers to an organism that lives on or in a host organism and derives nutrition from or at the expense of its host. For example, Chaga's disease is an inflammatory, infectious disease caused by the parasite Trypanosoma cruzi, which is transmitted by bloodsucking insects that causes damage to the heart and central nervous system.

The term “pattern recognition molecules” or “PRMs” as used herein refer to proteins recognizing PAMPs. Soluble PRMs include the collectins, acute phase proteins and NOD proteins. Membrane-bound PRMs are pattern recognition receptors.

The term “pattern recognition receptors” or “PRRs” refers to widely distributed membrane bound PRMs fixed in either the plasma membrane of a cell or in the membranes of its endocytic vesicles. PRRs include toll-like receptors (TLRs) and scavenger receptors. CLRs are a type of PRR; Dectin-1, CLEC9 and DEC-205 (lymphocyte antigen 75) are all examples of CLRs. Engagement of PRRs induces pro-inflammatory cytokines.

The term “peroxisome proliferator-activated receptors” or “PPARs” as used herein refers to fatty acid-activated transcription factors of nuclear hormone receptor superfamily that regulate energy metabolism. Three PPAR subtypes have been identified: PPARα, PPARγ, and PPARβ/δ. PPARα and PPARδ are highly expressed in oxidative tissues and regulate genes involved in substrate delivery and oxidative phosphorylation (OXPHOS) and regulation of energy homeostasis. PPARγ is more important in lipogenesis and lipid synthesis, with highest expression levels in white adipose tissue (WAT). As key regulators of metabolism, PPARs guide the differentiation, expansion and fate commitment of various immune cell types. For example, In macrophages, PPARγ regulates polarization, maturation, epigenetics and metabolism [Christofides, A. et al., Metabolism (2021) 114: 154338, citing Szanto, A. et al., Immunity (2010) 33: 699-712; Odegaard, J I et al., Nature (2007) 447: 1116-20; Daniel, B. et al., Immunity (2018) 49: 615-26; Schneider, C. et al., Nat. Immunol. (2014) 15: 1026-37; Stafeev, Y S et al., Bull Exp. Biol. Med. (2018) 165: 429-33]. In dendritic cells (DC), which have a key role in regulating immunity vs. anergy (in vitro) and tolerance (in vivo), PPARγ is a central regulator of functional maturation, thereby guiding the ability to induce immunogenic T cell responses vs. immune tolerance PPARγ is also critical for the regulation of adaptive immune cells; it is a major driver of a unique population of Treg that differentiate and accumulate in the visceral adipose tissue (VAT), and are implicated in the control of the inflammatory state of adipose tissue and, thereby, insulin sensitivity [Id., citng Cipolletta, D. et al. Nature (2012) 486: 549-53; Feuerer, M. et al. Nat. Med. (2009) 15: 930-9]. PPARα appears to potentiate the polarization of macrophages towards an anti-inflammatory phenotype and plays an important role in T cell responses and in the development of T cell-mediated autoimmune diseases, in a gender-specific manner [Id., citing Zhang, M A et al. Proc. Natl Acad. Sci. USA (2012) 109: 9505-10]. PPARβ/δ has been implicated in T cell development and function [Id.].

The term “plasma” as used herein refers to the fluid (noncellular) portion of circulating blood, and the fluid portion of lymph.

The term “polarization of immune cells” as used herein refers to a process in which immune cells adopt distinct programs and perform specialized functions in response to specific signals. For example, naïve CD4+ T, i.e., Th0, cells can differentiate into Th1, Th2, or Th17 cells in response to specific stimulating signals. Polarized Th cells play critical roles in the initiation, amplification and resolution or progression of type 1, type 2, or type 3 immunity. Similarly, ILCs can polarize into ILC1, ILC2, and ILC3 subpopulations to regulate associated immune responses. [Ma, Q. Front. Immunol. (2020) 11: 1060].

In response to various environmental cues (e.g., microbial products, damaged cells, activated lymphocytes) or under different pathophysiologic conditions, macrophages also can acquire distinct functional phenotypes via undergoing different phenotypic polarization [Wang, N. et al. Front. Immunol. (2014) 5: 614], citing O'Shea, J J and Paul, WE. Science (2010) 327 (5969): 1098-102].

First, the M1 phenotype is stimulated by microbial products or pro-inflammatory cytokines [IFN-γ, TNF, or Toll-like receptor (TLR) ligands], and the typical characteristics of M1 macrophages include high antigen presentation, high production of IL-12 and IL-23, and high production of nitric oxide (NO) and reactive oxygen intermediates (ROI) [Id., citing Verreck, F A et al. Proc. Natl Acad. Sci. USA (2004) 101 (13) 4560-5]. In contrast, M2-type responses are the “resting” phenotype and are observed in healing-type circumstances without infections. Such responses can also be further amplified by IL-4, IL-10, or IL-13. M2 macrophages are characterized by the upregulation of Dectin-1, DC-SIGN, mannose receptor, scavenger receptor A, scavenger receptor B-1, CD163, CCR2, CXCR1, and CXCR2 [Id., citing Martinez, F O et al. Annu. Rev. Immunol. (2009) 27: 451-83]. Instead of generating NO or ROI, M2 macrophages produce ornithine and polyamines through the arginase pathway [Id., citing Mantovani, A. et al. Trends Immunol. (2002) 23 (11): 549-55; Gordon, S. and Martinez, F O. Immunity (2010) 32 (5): 593-604].

Second, inflammatory M1 macrophages produce many other pro-inflammatory cytokines like TNFα, IL-1, IL-6, IL-12, Type I IFN, CXCL1-3, CXCL-5, and CXCL8-10 [Id., citing Sica, A. Mantovani, A. J. Clin. Invest. (2012) 122 (3): 787-95], while M2 macrophages generate anti-inflammatory cytokines such as IL-10 and very low level of pro-inflammatory cytokines such as IL-12 [Id., citing Mosser, DM. J. Leukac. Biol. (2003) 73 (2): 209-12]. Additional signatures of the M2 phenotype, such as YM1 (a member of the chitinase family) and FIZZ1 (found in inflammatory zone 1, RETNLA) have been identified [Id., citing Raes, G. et al. J. Immunol. (2005) 174 (11): 6561].

Third, M1 macrophages promote a Th1 response and possess strong microbicidal and tumoricidal activity, while M2 macrophages are involved in metazoan parasite containment and promotion of Th2 response, tissue remodeling, immune tolerance, and tumor progression [Id., citing Gordon, S. and Taylor, PR. Nat. Rev. Immunol. (2005) 5 (12): 953-64; Mantovani, A. et al. Eur. J. Immunol. (2011) 41 (9): 2522-5].

Canonical IRF/STAT signaling is a central pathway in modulating macrophage polarization. Activation of IRF/STAT signaling pathways by IFNs and TLR signaling will skew macrophage function toward the M1 phenotype (via STAT1), while activation of IRF/STAT (via STAT6) signaling pathways by IL-4 and IL-13 will skew macrophage function toward the M2 phenotype [Id., et al. [Id., et al. Sica, A. and Mantovani, A. J. Clin. Invest. (2012) 122 (30): 787-. Signals initiated by IL-10, glucocorticoid hormones, apoptotic cell-released molecules, and immune complexes can also profoundly affect macrophage functional statue [Id., citing Biswas, S K and Mantovani, A. Nat. Immunol. (2010) 11 (10): 889-96]. Macrophage polarization is also modulated by local microenvironmental conditions such as hypoxia [Id., citing Escribese, M M et al. Immunobiol. (2012) 217 (12): 1233-40]. More importantly, M1-M2 polarization of macrophage is a highly dynamic process and the phenotype of polarized macrophages can be reversed under physiological and pathological conditions [Id., citing Saccani, A. et al. Cancer Res. (2006) 66 (23): 11432-40; Guiducci, C. et al. Cancer Res. (2005) 65 (8): 3437-46]. In the course of various pathophysiological settings, the same signaling pathway can be involved in either M1 or M2 polarization of macrophages. The molecular mechanisms that govern the phenotype switch of macrophages, however, remains incompletely understood.

The term “pro-inflammatory” as used herein refers to promoting or causing inflammation.

The term “purification” and its various grammatical forms as used herein refers to a process of isolating or freeing from foreign, extraneous, or objectionable elements. The composition is nonetheless substantially pure in that it has been substantially separated from the substances with which it may be associated in living systems or during synthesis. As used herein, the term “substantially pure” refers purity of at least 75%, at least 80%, at least 85%, at least 90%, at least 95% or at least 99% pure as determined by an analytical protocol. Such protocols may include, for example, without limitation, flow cytometry, electrophoresis, small-RNA sequencing, quantitative PCR, nanoparticle tracking, electron microscopy, mass spectrometry, Western blotting, ELISA, and various metabolic assays.

The term “RAW 264.7” refers to a mouse macrophage cell line.

The terms “reactive oxygen species” or “ROS” or “oxygen radical” are used herein interchangeably to refer to a type of unstable molecule that contains oxygen and that easily reacts with other molecules in a cell. A build-up of reactive oxygen species in cells may cause damage to DNA, RNA, and proteins, and may cause cell death. Reactive oxygen species are free radicals.

The term “serum” as used herein refers to the fluid portion of the blood obtained after removal of the fibrin clot and blood cells.

The term “solution” as used herein refers to a homogeneous mixture of two or more substances. It is frequently, though not necessarily, a liquid. In a solution, the molecules of the solute (or dissolved substance) are uniformly distributed among those of the solvent.

The term “solvent” as used herein refers to a substance capable of dissolving another substance (termed a “solute”) to form a uniformly dispersed mixture (solution).

The term “Sterol Regulatory Element Binding Protein (SREBP)” as used herein refers to membrane bound transcription factors that control lipid metabolism in mammalian cells.

There are three isoforms of SREBPs: SREBP-1a and SREBP-1c, resulting from alternate splicing of the SREBP1 gene and preferentially activating genes in fatty acid synthesis; and SREBP-2, primarily activating genes in the cholesterol metabolism pathway, including enzymes of cholesterol biosynthesis, the LDL receptor, and mediators of cholesterol efflux.

As used herein, the term “stimulate” in any of its grammatical forms as used herein is meant to refer to inducing activation or increasing activity.

The term “stimulate an immune cell” or “stimulating an immune cell” as used herein is meant to refer to a process (e.g., involving a signaling event or stimulus) causing or resulting in a cellular response, such as activation and/or expansion, of an immune cell, e.g. a CD8+ T cell.

The terms “subject” or “individual” or “patient” are used interchangeably to refer to a member of an animal species of mammalian origin, including but not limited to, mouse, rat, cat, goat, sheep, horse, hamster, ferret, pig, dog, guinea pig, rabbit and a primate, such as, for example, a monkey, ape, or human.

The term “T lymphocyte” or “T-cell” as used herein refers to one of the two types of antigen-specific lymphocytes responsible for adaptive immune responses, the other being the B cells. T cells are responsible for cell-mediated adaptive immune reactions. They originate in the bone marrow but undergo most of their development in the thymus. The highly variable antigen receptor on T cells is celled the T-cell receptor and recognizes a complex of peptide antigen bound to major histocompatibility complex (MHC) molecules on cell surfaces. There are two main lineages of T cells: those carrying ap receptors and those carrying 76 T cell receptors. Effector T cells perform a variety of functions in an immune response; they always act by interacting with another cell in an antigen-specific manner. Some T cells activate macrophages, some help B cells produce antibody, and some kill cells infected with viruses and other intracellular pathogens.

The term “toll-like receptor” or “TLRs” as used herein refers to innate receptors on macrophages, dendritic cells and some other cells that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

Signaling by mammalian TLRs in various cell types induces a diverse range of intracellular responses that together result in the production of inflammatory cytokines, chemotactic factors, antimicrobial peptides, and the type I antiviral cytokines interferon α and interferon β. [Janeway's Immunology, 9th Ed. (2017) Garland Science, New York, at 92] Macrophages and neutrophils secrete lipid mediators of inflammation—prostaglandins, leukotrienes, and platelet-activating factor (PAF)—which are rapidly produced by enzymatic pathways that degrade membrane phospholipids.

The term “tumor necrosis factor” or “TNF” as used herein refers to a cytokine made by white blood cells in response to an antigen or infection, which induce necrosis (death) of tumor cells and possesses a wide range of pro-inflammatory actions. Tumor necrosis factor also is a multifunctional cytokine with effects on lipid metabolism, coagulation, insulin resistance, and the function of endothelial cells lining blood vessels.

The term “type 1 immunity” as used herein refers to a class of effector activities aimed at elimination of intracellular pathogens. It is characterized by Th1 cells and ILC group 1 cells, which secrete interferon (IFN)-γ, IL-2, and lymphotoxin-α. Type 1 responses protect against intracellular microbes through activated mononuclear phagocytes, i.e., M1 macrophages, and an array of proinflammatory cytokines, eicosanoids, and reactive oxygen species (ROS) and reactive nitrogen species (RNS), to stimulate acute inflammation and bacterial killing. Heightened type 1 responses cause excessive damage to lung tissue and contribute to disease pathogenesis, including releasing self-antigens that induce autoimmune reactions. [Ma, Q. Front. Immunol. (2020) 11: 1060.]

The term “type 2 immunity” as used herein refers to a class of effector activities aimed at elimination of parasites and promoting barrier and mucosal immunity. It consists of Th2 cells, ILC2s, and M2 macrophages, which secrete type 2 cytokines, such as IL-4, IL-5, IL-9, IL-10, and IL-13. These cytokines recruit and activate type 2 effector cells, including eosinophils, basophils, mast cells, and myofibroblasts. Some Th2 cells migrate to lymph node follicles and promote IgE class switch and B cell activation and hence are called follicular helper T cells (Thfs). Type 2 responses protect against helminth infection, venoms, and allergens under physiological conditions but, when dysregulated, lead to atopic responses, such as asthma and anaphylaxis. ILC2s can be activated in response to a wide range of stimuli; activated ILC2s secrete copious amounts of type 2 cytokines prior to Th2 activation [Ma, Q. Front. Immunol. (2020) 11: 1060, citing Pulendran, B. and Artis, D. Science (2012) 337: 431-5]. These findings suggest a mechanism by which type 2 responses can be initiated in the absence of apparent antigenic stimulation. While functionally, type 2 immunity has been traditionally associated with allergic responses, host defense against helminth infection, and tissue repair, a contemporary view of type 2 functions exists in which type 2 responses seemingly play a more general role in defense against noxious environmental stimuli besides mediating host immunosurveillance at barrier sites. [Id.]. In this context, type 2 reactions help eliminate, restrict, and neutralize noxious environmental substances and triggers, such as allergens, as well as repair tissue damage and minimize inflammation at surface tissue. [Id.] Key to the function of type 2 responses in tissue regeneration, wound healing, and suppression of type 1 inflammation is the production of transforming growth factor (TGF)-β by type 2 cells, such as M2 macrophages. [Id.] Additionally, ILC2s, eosinophils, and type 2 cytokines are vital regulators of adipose precursor number and fate and overall adipose tissue homeostasis [Id., citing Lee, M W, et al. Cell (2015) 160: 74-87]. This innate type 2 immune metabolic circuit regulates energy metabolism and, thereby, controls insulin sensitivity and lean physiology [Id., citing Brestoff, J R et al. Nature (2015) 519: 242-6]. Therefore, the view of the biology and scope of type 2 immunity has expanded considerably beyond the traditionally recognized type 2 responses [Lloyd, C M and Snelgrove, RJ. Sci. Immunol. (2018) 3: eaatl604].

The term “type 3 immunity” as used herein refers to a class of effector activities aimed at elimination of extracellular pathogens such as bacteria and fungi.

Central to these Type 1, type 2, and type 3 immunity/inflammation is the polarization of several major immune cells, including T lymphocytes, macrophages, and ILCs, induced by microbial signals, allergens, sterile insults, and microenvironmental cues from damaged tissue. [Ma, Q. Front. Immunol. (2020) 11: 1060.]

In addition to type 1, type 2, and type 3 responses, Treg lymphocytes can be enriched to regulate immune responses by controlling the polarization of Th1, Th2, and Th17 effector T (Teff) cells and hence the balance among these immune responses. [Ma, Q. Front. Immunol. (2020) 11: 1060.]

The term “type I interferons” as used herein refers to the antiviral interferons IFN-α and IFN-β. Almost all types of cells can produce IFN-α and IFN-β in response to activation of several innate sensors.

The term “type II interferon” as used herein refers to the antiviral interferon IFN-γ. IFN-γ is the sole type II interferon; the dominating biological role of IFN-γ seems to be stimulation of the adaptive immune system, primarily activation of T cells [Zhou, Z. et al. J. Virology (2007) 81 (14): 7749-58., citing Biron, C. A. (1994). Curr. Opin. Immunol. 6:530-538, Muller, U., et al. (1994) Science 264:1918-1921] IFN-γ is produced by activated Th1 cells and ILCIs, including NK cells, which are activated mainly through TLRs.

The term “white adipose tissue” or “WAT” as used herein refers to a heterogeneous tissue composed of lipid-filled adipocytes and several non-adipocyte cell populations, including endothelial cells, blood cells, uncharacterized stromal cells and adipocyte precursor cells.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either/both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates, which may need to be independently confirmed.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed.

Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Circulating Adipomes can Modulate Cardiac Morphology

According to some embodiments, circulating adipomes can modulate primary cardiomyocytes in vitro.

According to some embodiments, circulating adipomes can modulate cardiac morphology in a mouse model. According to some embodiments, in the mouse, the administration of circulating adipomes alters cardiac morphology as evidenced by altered left and right ventricle internal diameters, septal wall thickness and ejection fraction, compared to a infected control.

### Experimental Model and Study Participant Details

### Mammalian Cell Lines and Parasites Culturing

Murine 3T3-L1 preadipocytes and Human subcutaneous preadipocytes were purchased from ZenBio. The preadipocytes were differentiated into mature adipocytes by feeding adipocyte differentiation media (murine: for 3 days; and human: for 7 days) and further maintained in adipocyte maintenance media purchased from ZenBio until ready to be assayed. Adipocytes were used between 7-14 days (murine) and 14-21 days (human) post-initiation of differentiation. HFF-1 human foreskin fibroblast and RAW 264.7 murine macrophage cell lines were originally purchased from American Type Culture Collection (ATCC) and maintained as an adherent culture in Dulbecco's Modification of Eagle's Medium (DMEM) supplemented with 10% heat-inactivated fetal bovine serum (HI-FBS) and 1% penicillin/streptomycin. Adult Mouse Cardiomyocytes were purchased from Celprogen and cultured as a monolayer in flasks pre-coated with Adult Mouse Cardiomyocyte Cell Culture Extra-cellular Matrix. Cardiomyocytes were fed with Cardiomyocyte Primary Cell Culture Media with Serum and used for the experiment after 18 hours of plating. All the cell lines were cultured and maintained in a humidified incubator at 37° C. and 5% CO2. Trypomastigotes of T. cruzi (Brazil strain) were propagated in HFF cell line and maintained by serial passage in confluent monolayers of HFF cells in DMEM containing 10% HI-FBS and 1% penicillin/streptomycin.^{9,118} Trypomastigotes harvested from cell culture supernatants were washed 3 times in cold Dulbecco's phosphate-buffered saline (DPBS) without Calcium and Magnesium by centrifugation, counted and resuspended in warm DPBS at 105 parasites per mL and used for in vitro and in vivo infection experiments.

T. cruzi Infection In Vitro

Cultured murine and human adipocytes were infected with tissue culture-derived trypomastigotes of T. cruzi (Brazil strain) at a multiplicity of infection (MOI) of 3:1.^{10,17} 24 hours post infection, infected cells were washed twice with warm DPBS to remove excess, unbound parasites and replenished with fresh culture media and incubated for additional 48 hours. Two separate sets of cultured adipocytes (both murine and human) were also included: one set served as uninfected control and the other set was treated with 10 ng recombinant TNFα for 48 hours to induce apoptosis. At the experimental end points, the conditioned media (cell culture supernatant) from uninfected, TNFα-treated and T. cruzi infected groups of both murine and human adipocytes were collected for adipome isolation. The cells were harvested separately for protein (murine) and mRNA (human) analysis.

### Murine Model of Acute Chagas Disease

As males are more prone to develop Chagas cardiomyopathy, only male mice were used in this study. Male C57BL/6J mice, 6-7 weeks old (n=8), were infected intraperitoneally (i.p.) with 1×104 tissue culture-derived T. cruzi trypomastigotes.¹¹ A separate group of age- and sex-matched uninfected mice (n=8) were included as controls. Mice were housed at CDI animal research facility in sterilized filter top cages under 12-h light-dark cycle and humidity and temperature-controlled conditions. All animals had ad libitum access to water and rodent chow. Animals were routinely monitored for signs of illness for the entire duration of the study. At 20 days post infection (DPI), both infected and uninfected control mice were euthanized and tissues including heart, visceral fat pads (epididymal white adipose tissue) were harvested along with terminal blood collection.^{11,119,120} The experiment was repeated to isolate required amount of adipomes for the functional studies. All animal experiments were conducted in accordance with the National Institutes of Health Guide for the Care and Use of Laboratory Animals with approval from the Institutional Animal Care and Use Committee (protocol #282) of the Center for Discovery and Innovation (CDI)—Hackensack University Medical Center.

### Method Details

### Adipome Isolation and Functional Analysis

### In Vitro Adipocytes-Derived Adipomes

Adipomes released by cultured adipocytes were isolated using ExoQuick-TC Ultra EV isolation kit. The conditioned media from murine and human adipocytes (uninfected, TNFα-treated, T. cruzi infected groups) were centrifuged at 3,000×g for 20 minutes to remove parasites and cellular debris and the supernatants were transferred to new tubes. Additionally, the supernatants were passed through a 1.2 m syringe filter to remove any parasite contamination in samples. ExoQuick-TC exosome precipitation solution was added to the clarified supernatants at 1:5, mixed thoroughly by inverting the tubes and incubated overnight at 4° C. The samples were then centrifuged at 3,000×g for 10 minutes to pellet the precipitated EVs. The EV pellets were resuspended and purified by passing through the size exclusion chromatography (SEC) columns provided in the kit following manufacturer's instructions. These purified adipomes were treated on HFF (with human adipocytes-derived adipomes) and RAW (with murine adipocytes-derived adipomes) cells at 1:1 and 1:50 (cell to adipome ratio) for 48 hours and the cells were harvested for mRNA analysis. The adipome numbers were calculated based on the Spectradyne nCS1 measurements.

### In Vivo White Adipose Tissue (WAT)-Derived Adipomes

Adipomes from WAT were isolated and enriched using a combination of ExoQuick-TC Ultra EV isolation kit and Basic Exo-Flow Capture kit as follows. The WATs from infected and uninfected mice were processed separately inside the biosafety cabinet. The harvested tissues were placed in a 12-well plate and gently dissociated in presence of 500 μL cold 1×PBS (sterile) using sterile forceps and scalpel to liberate EVs present in the intercellular spaces. Upon dissociation, the biofluid was passed through 70 m cell strainer to remove large chunks of tissue and flushed with additional 500 μL of cold PBS. The biofluid was then centrifuged at 500×g for 10 mins to remove cell debris at 10° C. The supernatant was transferred to a new tube and re-centrifuged at 21,000×g for an hour at 10° C. After completion of the spin, the supernatant was carefully transferred again to a new tube without disturbing the pellet and/or the white precipitates smeared on the inner sides of the tube, if using fixed-angle rotor. The pellet/precipitate was resuspended in 250 μL PBS. Then, the ExoQuick-TC exosome precipitation solution was added to both the resuspended pellet and supernatant fraction at 1:5, mixed thoroughly by inverting the tubes and incubated overnight at 4° C. On the following day, the samples were centrifuged at 3,000×g for 10 minutes to pellet the precipitated EVs and further purified using the SEC columns as detailed above. The EVs from resuspended pellets were referred as large-EVs (L-EV) while the supernatant derived EVs were referred as small-EVs (S-EV). Next, L-adipomes and S-adipomes were enriched from L-EV and S-EV, respectively utilizing adipocytes-specific antibody cocktail (0.5 g/assay biotinylated mouse adiponectin antibody and 0.5 g/assay biotinylated mouse FABP4 antibody) and magnetic streptavidin 9.1 m Exo-Flow bead system following manufacturer's instructions with slight modification to improve recovery. Adipomes enriched from control uninfected L-EV and S-EV were referred to as CLA and CSA, respectively. Whereas adipomes from infected L-EV and S-EV were referred to as ILA and ISA, respectively. Washed bead-bound adipomes were sent for lipidomics analysis (Georgetown University Metabolomics Center). The protein from magnetic bead bound adipomes were extracted directly by on-bead lysis (using 0.25% Triton X-100 followed by sonication and used for method validation by western blot and qPCR analyses. The eluted adipomes were reprecipitated with ExoQuick-TC precipitation buffer for 2 hours at 4° C. to remove the elution buffer, centrifuged at 3,000×g for 20 minutes at 10° C., resuspended in 1×PBS and used for adipome characterization (size, distribution, concentration TME) and functional analyses in murine RAW macrophages and primary cardiomyocytes.

Plasma-derived adipomes. Adipomes were isolated from uninfected and T. cruzi infected mice plasma in a similar fashion as described above using ExoQuick Ultra EV isolation kit for serum/plasma with the addition of thrombin treatment. Plasma-derived Infection-associated L-adipomes (P-ILA) were injected to C57BL/6J mice and analyses including Cardiac Ultrasound imaging, histological examination of heart tissue and protein analysis were performed.

EXOCET assay. The amount of WAT-derived large-EVs and small-EVs were quantified using EXOCET Exosome Quantification kit following manufacturer's instructions. The assay measures the activity of Acetyl-CoA Acetylcholinesterase (AChE), an enzyme highly enriched in exosomes.¹²¹

### Immunofluorescence Analysis

Adipocyte staining. Murine 3T3-L1 adipocytes were grown and differentiated on glass coverslips and treated with 10 ng recombinant TNFα for 24 hours. The cells were fixed overnight with 4% paraformaldehyde for 2 hours, washed three times in PBS and blocked with 10% goat serum for 30 minutes at room temperature to minimize non-specific adsorption of antibodies. The cells were then stained with a combination of mouse monoclonal Adiponectin and rabbit monoclonal Annexin V primary antibodies in one set and goat polyclonal FABP4 and rabbit monoclonal Annexin V primary antibodies in another set for an hour at room temperature. Followed by secondary staining with an appropriate Alexa Fluor antibody combination: Goat anti-mouse IgG Alexa Fluor 488 and Goat anti-rabbit IgG Alexa Fluor 594 for the first set and Donkey anti-goat IgG Alexa Fluor 594 and Goat anti-rabbit IgG Alexa Fluor 488 for the second set. The coverslips were washed and mounted with VECTASHIELD HardSet Antifade Mounting Medium. Samples were then imaged on Nikon Eclipse Ti2 Epi-fluorescence microscopy (Nikon Instruments Inc).

Adipome staining. WAT-derived adipomes were bound to magnetic streptavidin 9.1 m Exo-Flow beads conjugated to Adiponectin/FABP4 antibody as detailed in adipome isolation section. The adipome-bound beads were washed twice with PBS and parted in two halves. One fraction was stained with mouse monoclonal Adiponectin antibody followed by secondary staining with Goat anti-mouse IgG Alexa Fluor 488. The other fraction was stained with rat monoclonal FABP4 antibody followed by secondary staining with Goat anti-rat IgG Alex Fluor 488. A third tube containing untouched magnetic streptavidin beads alone was stained with a mix of Goat anti-mouse and anti-rat IgG Alexa Fluor 488 and used as control. The stained beads suspended in PBS were spotted on a glass slide and mounted using coverslip with VECTASHIELD HardSet Antifade Mounting Medium. The beads were imaged on Nikon Eclipse Ti2 Epi-fluorescence microscopy equipped with a Nikon Plan Fluor 100×/1.30na objective.

Nanoparticle Tracking. Spectradyne nCS1 instrument was used to evaluate the size and concentration of adipocytes-derived EVs/adipomes. Cultured murine and human adipocytes-derived adipomes were analyzed using C-900 cartridges with a size detection range of 130-900 nm particle diameters. WAT-derived adipomes were analyzed using C-2000 cartridges with size detection range of 250-2000 nm. In all cases, samples were prepared by mixing 9 μL of purified adipomes with 1 μL of 0.02 m filtered 10×PBS-T to increase sample conductivity. Out of which, 5 μL samples were loaded onto the cartridges and primed in the instrument using 0.2 m filtered 1×PBS-T. Particle acquisition was performed on continuous mode until the count reaches a set stop point of 2000 particles with <2.5% statistical error. The acquired stats files were analyzed for particle size distribution and concentration (per mL) using Spectradyne Data Viewer software and to generate peak-filtered, background subtracted CSD plots.

Transmission electron microscopy. Transmission electron microscopy (TEM) images of L-adipomes and S-adipomes were obtained at the analytical imaging facility—Albert Einstein College of Medicine. Adipome samples were fixed with 2% Glutaraldehyde in 0.1 M Phosphate Buffer and stored at 4° C. 400 mesh, carbon only grids were plasma cleaned using a Tergeo-EM Plasma Cleaner (PIE Scientific). Fixed adipome suspension were adsorbed onto grids for 10 mins and wicked dry. Samples were then negatively stained with 1% aqueous uranyl acetate and viewed on a Tecnai 20 transmission electron microscope (ThermoFisher Scientific) operating at a voltage of 120 kV. Acquired images were saved in high resolution TIFF format.^{122,123}

### Cardiac Ultrasound Imaging

Male C57BL/6J mice, 6-7 weeks old (n=6) were treated with P-ILA (1×10E9 adipomes suspended in 40 ul 1×PBS) or vehicle (1×PBS) through ultrasound-guided intracardiac injections using a Vevo 3100 preclinical imaging system. Ultrasound-guided injections were performed twice a week to administer adipomes/vehicle into left ventricle wall.¹²⁴ Cardiac Ultrasound Imaging of the hearts were performed 4 days after the second dose of adipome administration.11 In another experiment, mice (n=10) were first infected intraperitoneally with 1×10E3 trypomastigotes of T. cruzi.¹¹ Infected mice were then treated with P-ILA (1×10E9 adipomes suspended in 40 ul 1×PBS) or vehicle (1×PBS) after 35 days post infection (DPI) by intracardiac injections. A separate group of age- and sex-matched uninfected mice (n=8) were included as controls and treated with either P-ILA or vehicle. B-mode, M-mode, and pulse wave Doppler image files were collected from both the parasternal long-axis and short-axis views. Morphometric measurements were determined using the Vevo LAB ultrasound analysis software.

Histological analysis. Mice heart tissues were collected 10 days after the first β-ILA administration. A portion of the freshly isolated tissues were fixed with 10% neutral-buffered formalin for a minimum of 48 hours and then embedded in paraffin wax. Hematoxylin and eosin and Picrosirius red staining were performed, and the images were captured.¹¹ Immunohistochemistry (IHC) was performed on the formalin-fixed heart sections using rabbit polyclonal ANP antibody and rabbit polyclonal TNFα antibody with a dilution of 1:250 and 1:200, respectively, followed by biotinylated secondary antibody using VECTASTAIN Elite ABC-HRP kit. The sections were then washed and incubated with peroxidase substrate and counterstained with hematoxylin. Four to six images per section were captured and quantification of DAB positive staining intensity was performed using the image processing and analysis tool ImageJ.

Immunoblotting analysis. Murine 3T3-L1 adipocytes (uninfected, TNFα-treated and T. cruzi infected) were washed twice with cold PBS followed by incubating on ice with 400 L of 1× lysis buffer containing Pierce protease inhibitor cocktail for 10 minutes. The cells were then scraped off and transferred to a sterile tube and sheared by passing through 28G1/2 insulin syringe for ten times. The cell lysates were centrifuged for 20 minutes at 14,000×g in a cold microfuge and the clarified supernatants were recovered in new tubes. Mice WAT and heart tissues (uninfected, infected, and adipome treated) were homogenized using a handheld homogenizer after adding an appropriate volume of 1× lysis buffer with protease inhibitor cocktail. The homogenates were then incubated on ice for 10 minutes followed by centrifugation and supernatant recovery as mentioned above. The protein concentration was quantified using Pierce BCA protein quantification kit. 30 g total protein from each sample were treated with 2-Mercaptoethanol, reducing agent and heat denatured at 100° C. for 5 minutes before resolving on SDS-PAGE. Protein from cultured adipocytes-derived and WAT-derived adipomes were prepared by lysing the samples with 0.25% Triton X-100 v/v and sonicating the sample tubes in an ultrasonic water bath for 10 minutes on high frequency setting. Adipome protein concentration was quantified using Pierce Micro BCA protein assay kit. Samples were reduced, heat denatured and resolved on SDS-PAGE as mentioned earlier. PageRuler Plus Prestained protein ladder was used as size standards in SDS-PAGE. The proteins were then transferred onto nitrocellulose membrane for immunoblot analysis. Primary antibodies against Caspase 7, Cleaved Caspase 7, Annexin V, Phospho-Perilipin 1, Adiponectin, Perilipin 1, FABP4, β1-Adrenergic Receptor, CHOP, TNFα, IFNγ, IL6, F4/80, SREBP, PPARα, SDHA, Cytochrome c, COX IV, HSP60, BiP, Phospho-eIF2α, PDI, and BNIP3 were used to detect the expression of corresponding proteins. β-Actin and Guanosine nucleotide dissociation inhibitor were used as appropriate control to assess equal protein loading. Horseradish peroxidase (HRP)-conjugated anti-mouse immunoglobulin, HRP-conjugated anti-rabbit immunoglobulin and HRP-conjugated anti-rat immunoglobulin were used as appropriate secondary antibodies to detect chemiluminescent signal using SuperSignal West Pico PLUS or SuperSignal West Atto Ultimate Sensitivity substrate on the Invitrogen iBright Imaging Systems. All the densitometric analyses of the target protein bands on the immunoblots were analyzed with the Image Studio lite package Ver 5.2 (LI-COR Biosciences).

RNA extraction and quantitative PCR. Cultured human adipocytes (uninfected, TNFα-treated and T. cruzi infected) and adipomes treated human fibroblasts, RAW macrophages and murine cardiomyocytes were subjected to total RNA isolation using RNeasy mini kit according to manufacturer's instructions. 0.5 to 1 g of total RNA was used for reverse transcription and cDNA synthesis using RT² First Strand Kit after genomic DNA elimination as per manufacturer's instructions. The cDNA samples were diluted at 1:5 and quantitative PCR (qPCR) experiments were performed using RT² SYBR Green ROX Mastermix following manufacturer's instructions. All assays were performed on Applied Biosystems QuantStudio 3 Real-time PCR System according to the following cycling conditions: 10 minutes at 95° C. (1 cycle, HotStart DNA Taq Polymerase activation), followed by 15 seconds at 95° C. and 1 minute at 60° C. (40 cycles, PCR amplification and data collection). Dissociation (melting) curve analysis was added to the run set up by enabling default melting curve program to verify PCR specificity. Data analysis was performed normalized to the expression of HPRT and/or GAPDH using the ΔΔCT method and the mRNA expression (fold change) levels were plotted as bar graphs.

Custom RT2 Profiler Real-Time PCR array for mouse genes involved in adipogenesis, lipogenesis, cellular metabolism-specific signaling molecules and markers of immune and inflammatory responses was used to analyze gene expression in RAW macrophages treated with L- and S-adipomes of uninfected and T. cruzi infected mice for 48 hours. Data analysis was performed normalized to the expression of Hprt using the ΔΔCT method and statistical analysis was performed as suggested using RT2 qPCR Assay Data Analysis 1808 Spreadsheet from Qiagen.^{26,68}

Total RNA from adipomes were extracted using TRIzol LS reagent and purified using miRNeasy Mini kit following manufacturer's instructions. The total yield was used for reverse transcription and cDNA synthesis as mentioned above. PCR was performed with undiluted cDNA samples to analyze the presence of adipogenic genes, apoptotic and EV-specific marker genes within adipomes. The PCR product was resolved on 2% agarose gel and imaged using the Invitrogen iBright Imaging Systems.

For quantitative determination of parasite load in P-ILA treated heart, a standard curve PCR was performed using the T. cruzi DNA standard (ranging from 31.2 pg to 0.002 pg) along with DNA samples extracted from mice heart tissue.17 Complementary DNA made from RNA isolated from T. cruzi infected 3T3-L1 adipocytes (as positive control) and P-ILA treated cardiomyocytes were also included in the PCR.

Lipidomics analysis. All LC-MS grade solvents including acetonitrile and water were purchased from Fisher Scientific. High purity formic acid (99%) was purchased from Thermo Fisher Scientific. EquiSPLASH LIPIDOMIX quantitative mass spec internal standard and 15:0-18:1-d7-PA, C15 Ceramide-d7 (d18:1-d7/15:0) and 18:1 Chol (D7) ester were purchased from Avanti polar lipids. Internal standard for free fatty acid (FFA), dihydroceramides (DCER), hexosylceramides (HCER), lactosylceramides (LCER) were purchased from Sciex as Lipidyzer platform kit. This method was designed to measure 21 classes of lipid molecules which includes diacylglycerols (DAG), chloesterol esters (CE), sphingomyelins (SM), phosphtatidylchloine (PC), triacylglycerols (TAG), free fatty acids (FFA), ceramides (CE), dihydroceramides (DCER), hexosylceramide (HCER), lactosylceramide (LCER), phosphatidylethanolamine (PE), lysophosphtatidylchloine (LPC), lysophosphatidylethnolamine (LPE), phosphatidic acid (PA), lysophosphatidic acid (LPA), phosphatidylinositol (PI), lysophosphotidylinositol (LPI), phosphatidylglycerol (PG), acylcarnitines and phosphatidylserine (PS) using QTRAP 5500 LC-MS/MS System (Sciex) as detailed below.

The adipomes (CLA, ILA, CSA and ISA) bound to magnetic beads were suspended in 1×PBS (40 ul). Repeated freeze thaw cycles followed by sonication for 30 sec was performed to release the contents of adipome cargoes into suspension. To the above solution, 100 L of chilled isopropanol containing internal standards for lipids was added and the samples were vortexed and kept on ice for 20 minutes. Samples were incubated at −20° C. for 20 minutes and then centrifuged at 13,000 rpm for 20 minutes at 4° C. The supernatant was transferred to MS vial for LC-MS analysis. 5 μL of each sample was injected onto a Xbridge amide 3.5 m, 4.6×100 mm (waters) using SIL-30 AC auto sampler (Shimazdu) connected with a high flow LC-30AD solvent delivery unit (Shimazdu) and CBM-20A communication bus module (Shimazdu) online with QTRAP 5500 (Sciex) operating in positive and negative ion mode. A binary solvent comprising of acetonitrile/water 95/5 with 10 mM ammonium acetate as solvent A and acetonitrile/water 50/50 with 10 mM ammonium acetate as solvent B was used for the resolution. Lipids were resolved at 0.7 mL/min flow rate, initial gradient conditions started with 100% of solvent A, shifting towards 99.9% of solvent A over a time period of 3 minutes, 94% of solvent A over a time period of 3 minutes and 25% of solvent A over a period of 4 minutes. Finally, washing with 100% of B for 6 minutes and equilibrating to initial conditions (100% of solvent A) over a time period of 6 minutes using auto sampler temperature 15° C. and oven temperature 35° C. Source and gas setting were as follow: curtain gas=30, CAD gas=medium, ion spray voltage=5.5 kV in positive mode and −4.5 kV in negative mode, temperature=550° C., nebulizing gas=50 and heater gas=60. The data were normalized to respective internal standard area for each class of lipid and processed using MultiQuant 3.0.3 (Sciex). The quality and reproducibility of LC-MS data was ensured using a number of measures. The column was conditioned using the pooled QC samples initially and were also injected periodically to monitor shifts in signal intensities and retention time as measures of reproducibility and data quality of the LC-MS data. NIST plasma sample prepared in the same manner was also ran alongside to check for the instrumental variance. Additionally, blank solvent runs were performed between set of samples to minimize carry-over effects. Lipidomics data were provided in the form of normalized response which was area under the curve for each metabolite divided by internal standard area. Lipid data was preprocessed using signal/noise>20:1 and retention time (RT) tolerance 5 seconds. Analysis yielded 287 reliable features in the adipome samples under study. The quality of data was monitored based on coefficient of variation (CV) of each lipid in pooled QC samples, the lipids having CV>20% were filtered off. The figures were generated using MetaboAnalyst 5.0.

### Magnetic Resonance Imaging (MRI)

Cardiac gated MRI was performed on murine model of acute Chagas disease along with uninfected controls using a 9.4T Varian Direct Drive animal magnetic resonance imaging and spectroscopic system (Agilent Technologies).8,68

### Seahorse XF96 Cell Metabolic Analysis

The mitochondrial function of cardiomyocytes upon plasma-derived adipome treatments was assessed by Seahorse XFe96 analyzer, using the Seahorse XF Cell Mito Stress Test kit. Mouse cardiomyocytes were treated with vehicle, β-CLA, and P-ILA at 1:20 (cell to adipome) ratio for 48 hours before assessing their mitochondrial function. A day prior to the assay, the adipome treated cells were plated on to a Cell-Tak coated XFe96 cell culture microplate at a seeding density of 20,000 cells/well, supplemented with cardiomyocyte culture media and incubated overnight at 37° C. in 5% CO2 incubator. The XFe96 sensor cartridge was hydrated overnight with sterile water in a humidified non-Cβ2 37° C. incubator and then submerged in pre-warmed XF-calibrant for 45-60 min prior to loading drug ports. On the day of the assay, the cells were supplied with Seahorse assay medium (XF DMEM medium pH 7.4, 10 mM XF Glucose, 2 mM XF Glutamine, 1 mM XF Pyruvate). For determination of mitochondrial respiration, 1.5 μM Oligomycin, 0.5 μM FCCP, and 0.5 M Rotenone/AA were prepared in assay medium and added to the ports A, B, and C on the sensor cartridge, respectively. Mitochondrial function was assessed in real time following the injection of Oligomycin, FCCP, and Rotenone/AA according to the default measurement protocol. Data were normalized to nuclear content by staining the live cells with Hoechst 33342 after the assay and fluorescence cell counting using Biotek Cytation C10. The normalized data was then exported to the report generator to create graphs.

### Quantification and Statistical Analysis

Statistical analyses were performed using GraphPad Prism Ver 10.0 (GraphPad Software, LLC) or Microsoft Excel (Microsoft Corp). Comparisons between groups were made using Two-Way ANOVA and unpaired Student's t-test as appropriate for western blotting data analysis. For custom RT2 Profiler array, the analysis was performed using Qiagen RT2 qPCR Assay Data Analysis 1808 Spreadsheet and the p values were calculated based on Student's t-test. Fold-regulation (up or down) was plotted on Microsoft Excel. Statistical analysis of other qPCR experiments was performed using Microsoft Excel and the comparisons between the groups were analyzed using unpaired Student's t-test. Significant differences were reported for p values between ≤0.0001 and <0.05 with symbols (*/#) as appropriate. Data represent means S.E.M. For lipidomics, statistical analysis between the groups was done using MetaboAnalyst 5.0.

### STAR Methods

Key Resources Table

| Reagent or Resource | Source | Identifier |
|---|---|---|
| Goat Polyclonal Anti-Mouse | R & D Systems | Cat#BAF1119, RRID: |
| Adiponectin, Biotin | | AB_227357 |
| Conjugated | | |
| Mouse Monoclonal Anti- | Abcam | Cat#ab22554; RRID: |
| Adiponectin | | AB_447152 |
| Rabbit Polyclonal Anti- | BioVision | Cat#5901-250; RRID: |
| Adiponectin | | AB_442498 |
| Rabbit Monoclonal Anti- | Signalway Antibody | Cat#EC49511-Biotin; RRID: |
| Fabp4; Biotin Conjugated | | AB_3095322 |
| Goat Polyclonal Anti-Mouse | R & D Systems | Cat#AF1443, RRID: AB- |
| Fabp4 | | 2102444 |
| Rat Monoclonal Anti-Fabf4 | R& D Systems | Cat#MAB3150; RRID: |
| | | AB_30095323 |
| Rabbit Monoclonal Anti- | Abcam | Cat#AB108194; RRID: |
| Annexin-V | | AB_10863755 |
| Rabbit Polyclonal Anti- | Cell Signaling Technology | Cat#855; RRID: |
| Annexin V | | AB_10950499 |
| Rabbit Monoclonal Anti- | Cell Signaling Technology | Cat#12827; RRID: |
| Caspase-7 | | AB_2687912 |
| Rabbit Monoclonal Anti- | Cell Signaling Technology | Cat#8438; RRID: |
| Cleaved Caspase-7 | | AB_11178377 |
| Mouse Monoclonal Anti- | Vala Science | Cat#4856; RRID: |
| Phospho-Periplin 1 | | AB_2909466 |
| Rabbit Polyclonal Anti- | Thermo Fisher Scientific | Cat# PAS-72921; RRID: |
| Perilipin 1 | | AB_2718775 |
| Rabbit Polyclonal Antiβ1- | Cell Signaling Technology | Cat#12271; RRID: |
| Adrenergic Receptor | | AB_2797865 |
| Mouse Monoclonal Anti- | Cell Signaling Technology | Cat#2895; RRID: |
| CHOP | | AB_2089254 |
| Rabbit Polyclonal Anti-TNF- | Cell Signaling Technology | Cat#3707; RRID: |
| α | | AB_2240625 |
| Rabbit Polyclonal Anti-IFNγ | Bioss | Cat#bs-0480R; RRID: |
| | | AB_10857066 |
| Mouse Monoclonal Anti-IL6 | Proteintech | Cat#64146-1-Ig; RRID: |
| | | AB_2881543 |
| Rat Monoclonal Anti-F4/80 | Novus Biologicals | Cat#NB600-404; |
| | | AB_10003219 |
| Rabbit Polyclonal Anti- | Abcam | Cat#28481; RRID: |
| SREBP1 | | AB_778069 |
| Mouse Monoclonal Anti- | Thermo Fisher Scientific | Cat#ma1-822; RRID: |
| PPARα | | AB_2165745 |
| Rabbit Monoclonal Anti- | Cell Signaling Technology | Cat#11998; RRID: |
| SDHA | | AB_2750900 |
| Rabbit Monoclonal Anti- | Cell Signaling Technology | Cat#4280; RRID: |
| Cytochrome C | | AB_10695410 |
| Rabbit Monoclonal Anti- | Cell Signaling Technology | Cat#4850 RRID: |
| COX IV | | AB_2085424 |
| Rabbit Monoclonal Anti- | Cell Signaling Technology | Cat#12165; RRID: |
| HSP60 | | AB_2636980 |
| Rabbit Monoclonal Anti-BiP | Cell Signaling Technology | Cat#3177; RRID: |
| | | AB_2119845 |
| Rabbit Monoclonal Anti- | Cell Signaling Technology | Cat#3398; RRID: |
| Phospho-EIF2α | | AB_2096481 |
| Rabbit Monoclonal Anti-PDI | Cell Signaling Technology | Cat#3501; |
| | | RRID: AB_2156433 |
| Rabbit Polyclonal Anti- | Cell Signaling Technology | Cat#3769; RRID: |
| BNIP3 | | AB_1159284 |
| Rabbit Polyclonal Anti-ANP | Thermo Fisher | Cat#711569; RRID: |
| | | AB_2662341 |
| Rabbit Polyclonal Anti-β- | Cell Signaling Technology | Cat#4967; RRID: |
| Actin | | AB_330288 |
| Rabbit Polyclonal Anti-GDI1 | Thermo Fisher | Cat#71-0300; |
| | | RRID: ab_2533970 |
| Horse Anti-Mouse IgG, HRP- | Cell Signaling Technology | Cat#7076; |
| Conjugated | | RRID: AB_330924 |
| Goat Anti-Rabbit IgG, HRP- | Cell Signaling Technology | Cat#7074; RRID: |
| Conjugated | | AB_2099233 |
| Goat Polyclonal Anti-Rat | Jackson ImmunoResearch | Cat#112-035-003; RRID: |
| IgG, HRP Conjugated | Labs | AB_238128 |
| Goat Polyclonal Anti-Mouse | ThermoFisher Scientifid | Cat#A-11001: RRID: |
| IgG, Alexa Fluor ™ 488 | | AB_2534069 |
| Goat Polyclonal Anti-Rabbit | Thermo Fisher Scientific | Cat#11012; RRID: |
| IgG, Alexa Fluor ™ 594 | | AB_2534079 |
| Donkey Polyclonal Anti-Goat | Thermo Fisher Scientific | Cat#A-11058; RRID: |
| IgG, Alexa Fluor ™ 594 | | AB_2534105 |
| Goat Polyclonal Anti-Rabbit | Thermo Fisher Scientific | Cat#A-1108; RRID: AB- |
| IgG, Alexa Fluor ™ 488 | | 143165 |
| Goat Polyclonal Anti-Rat | Thermo Fisher Scientific | Cat#11006; RRID: |
| IgG, Alexa Fluor ™ 488 | | AB_2534074 |

Chemicals, Peptides And Recombinant Proteins

| | | |
|---|---|---|
| 3T3L1 Differentiation | ZenBio | Cat#DM-2-L1 |
| Medium | | |
| 3T3-L1 Adipocyte Medium | ZenBio | Cat#AM-1-L1 |
| Subcutaneous Preadipocyte | ZenBio | Cat#DM-2 |
| Differentiation Medium | | |
| Subacute Adipocyte | ZenBio | Cat#AM-1 |
| Maintenance Medium | | |
| DMEM | Corning | Cat#10-013-CV |
| Fetal Bovine Serum, Heat | Thermo Fisher Scientific | Cat#16140071 |
| Inactivated | | |
| Penicillin-Streptomycin | Thermo Fisher Scientific | Cat#15140122 |
| (10000 U/Ml) | | |
| DPBS | Thermo Fisher Scientific | Cat#14190144 |
| Recombinant Mouse TNFα | BioLegend | Cat#718004 |
| Glutaraldehyde 2% | Electron Microscopy | Cat#1653-05 |
| | Sciences | |
| Trizol LS Reagent | Thermo Fisher Scientific | Cat#10296028 |
| RT²SYBER Green ROX | Qiagen | Cat#330523 |
| qPCR Mastermix | | |
| Cell Lysis Buffer | Thermo Fisher Scientific | Cat#9803 |
| Pierce Protease Inhibitor | Thermo Fisher Scientific | Cat#A32963 |
| Tablets | | |
| Triton X-100 | Thermo Fisher Scientific | Cat#85111 |
| Pageruler Plus Prestained | Thermo Fisher Scientific | Cat#26619 |
| Protein Ladder | | |
| Scientific Supersignal West | Thermo Fisher Scientific | Cat#34580 |
| Pico PLUS | | |
| Chemiluminescent Substrate | | |
| Supersignal West Atto | Thermo Fisher Scientific | Cat#A38556 |
| Ultimate Sensitivity | | |
| Chemiluminescent Substrate | | |
| Normal Goat Serum (10%) | Thermo Fisher Scientific | Cat#50062Z |
| Formalin Solution, Neutral | Millipore Sigma | Cat# |
| Buffered, 10% | | |
| Hematoxylin QA | Vector Laboratories | Cat# |
| Counterstain | | |
| VECTASHIELD Hardset | Vector Laboratories | Cat# |
| Antifade Mounting Medium | | |
| Cell-Tak | Corning | Cat# |
| Seahorse XF DMEM Assay | Agilent | Cat# |
| Medium Pack | | |
| Hoechst 33342 Solution | Thermo Fisher Scientific | Cat# |

Critical Commercial Assays

| | | |
|---|---|---|
| ExoQuick ULTRA EV | System Biosciences | Cat#EQUILTRA-20TC-1 |
| Isolation Kit for Tissue | | |
| Culture Media | | |
| Exoquick ULTRA EV | System Biosciences | Cat#EQUILTRA-20A-1 |
| Isolation Kit For Serum And | | |
| Plasma | | |
| Thrombin Plasma Prep | System Biosciences | Cat#TMEXO-1 |
| Basic Exo-Flow Capture Kit | System Biosciences | Cat#CSFLOWBASICA-1 |
| Exoquick-TC | System Biosciences | Cat#EXOTC10A-1 |
| Exocet Exosome Quantitation | System Biosciences | Cat#EXOCET96A-1 |
| Kit | | |
| Rneasy Mini Kit | Qiagen | Cat#74106 |
| MiRNeasy Mini Kit | Qiagen | Cat#217004 |
| RT² First Strand Kit | Qiagen | Cat#330404 |
| Pierce BCA Protein Assay | ThermoFisher Scientific | Cat#23225 |
| Kit | | |
| Pierce Micro BCA Protein | ThermoFisher Scientific | Cat#23235 |
| Assay Kit | | |
| VECTASTAIN Elite ABC- | Vector Laboratories | Cat#PK-6101 |
| HRP Kit, Peroxidase (Rabbit | | |
| Igg) | | |
| Impact DAB Substrate Kit, | Vector Laboratories | Cat#SK-410S |
| Peroxidase (HRP) | | |
| Seahorse XF Cell Mito Stress | Agilent | Cat#103015-100 |
| Test Kit | | |

Experimental Models, Cell Lines

| | | |
|---|---|---|
| Mouse: 3T3-L1 | ZenBio | SP-L1-F |
| Preadipocytes | | |
| Human: Subcutaneous | ZenBio | SP-F-1 |
| Preadipocytes | | |
| Human: HFF-1 cells | ATCC | SCRC-1041 |
| Mouse: RAW 264.7 | ATCC | TIB-71 |
| macrophages | | |
| Mouse: Cardiomyocytes | Celprogen | Cat#11041-14 |

Experimental models: organisms/strains

| | | |
|---|---|---|
| Mouse: C57BL/6J | The Jackson Laboratory | RRID: IMSR_JAX: 000664 |

Software and algorithms

| | |
|---|---|
| ImageJ | Schneider et al. ¹¹⁶ |
| Spectradyne Data Viewer | Spectradyne |
| Vevo LAP 5.8.2 | FUJIFILM VisualSonics |
| RT² qPCR Assay Data | Qiagen |
| Analysis 1808 Spreadsheet | |
| Image Studio Lite Ver. 5.2 | LI-COR Biosciences |
| GraphPad Prism Ver. 10 | GraphPad Software |
| Microsoft Excel | Microsoft Corp. |
| Seahorse Wave Desktop | Agilent |
| Software | |
| MultiQuant 3.0.3 | AB Sciex |
| MetaboAnalyst 5.0 | Pang, et al.¹¹⁷ |

Other

| | | |
|---|---|---|
| Trypomastigotes of | Combs et al. ¹⁷ | |
| Trypanosoma cruzi (Brazil | | |
| strain | | |
| Whatman ® Puradisc 30 | MilliporeSigma | Cat#WHA 10462260 |
| syringe filters) | | |
| Custom RT2 Profiler PCR | Qiagen | Cat#330171 |
| Array | | |
| Seahorse XFe96/XF Pro | Agilent | Cat#103793-100 |
| FluxPak Mini | | |

### Results

In our previous studies, we reported several key findings, including: (i) a significant correlation between the loss of body fat (adipocytes) and ventricular dilation,¹⁷ (ii) an elevated risk of cardiomyopathy during both acute and chronic stages and a correlation with adipocyte apoptosis,¹¹ and (iii) the involvement of adipogenic/lipogenic signaling associated with ER stress in the pathogenesis of chronic dilated cardiomyopathy in T. cruzi-infected mice.^{9,10} Together, these data suggested a link between adipocyte apoptosis and the pathogenesis of cardiomyopathy. However, the mechanism by which dying adipocytes may contribute to the risk of cardiomyopathy was not well understood. In this study, we investigated whether extracellular vesicles released by adipocytes under pathological conditions elevate the risk of cardiomyopathy by regulating heart muscle cells. We also proposed that these adipomes may exert paracrine and endocrine effects on various cell types in the myocardium, including resident and infiltrated immune cells. To systematically test our hypothesis, we isolated adipomes from cultured adipocytes and developed an innovative method to isolate circulating and AT-derived adipomes from mice acutely infected with T. cruzi. Subsequently, we conducted comprehensive in vitro, ex vivo, and in vivo functional analyses. The detailed results are provided later in the Discussion.

T. cruzi infection of cultured adipocytes leads to apoptosis and the release of adipomes To test our hypothesis, we investigated whether the T. cruzi infection of adipocytes during the early stage of infection (when most trypomastigotes invade adipocytes in culture, transform into amastigotes, and initiate replication) triggers adipocyte apoptosis and induces the release of extracellular vesicles (adipomes). We used cultured murine and human adipocytes infected with T. cruzi (MOI 3:1) for 72 h and analyzed protein and mRNA levels of apoptosis markers in murine and human adipocytes, respectively. The levels of apoptosis marker, cleaved caspase 7 was measured in cell lysates from T. cruzi-infected and uninfected murine 3T3-L1 differentiated adipocytes. As a positive control for apoptosis, we treated 3T3-L1 adipocytes with TNFα for 48 h.²³ Immunoblotting analysis revealed significantly higher levels of cleaved caspase in T. cruzi-infected and TNFα-treated adipocytes compared to uninfected cells, indicating that T. cruzi infection induces apoptosis in adipocytes (FIG. 1A). Similarly, qPCR analysis demonstrated a significant increase in CASP3 gene expression in T. cruzi-infected human adipocytes compared to uninfected cells (FIG. 1B). The levels of necroptotic gene markers such as RIPK3 and MLKL were not altered in T. cruzi-infected cells but were significantly increased in TNFα-treated cells compared to untreated human adipocytes (FIG. 1B).

Next, we investigated whether T. cruzi infection-induced apoptosis resulted in the release of adipocyte-specific extracellular vesicles (adipomes). We isolated adipomes from the conditioned medium using the Exoquick-TC Ultra EV isolation kit (FIG. 10) and quantified their size distribution and concentration using a Spectradyne nCS1 instrument. The size of adipomes ranged between 200 nm and 1100 nm for both cultured 3T3-L1 murine adipocytes and human adipocytes (FIG. 1C and FIG. 1D). The adipomes derived from uninfected and T. cruzi-infected 3T3-L1 adipocytes showed two distinct peaks corresponding to sizes ranging from 200 nm to 400 nm and 600 nm-1000 nm, whereas the adipomes from TNFα-treated 3T3-L1 adipocytes were mostly between 300 nm and 500 nm (FIG. 1C). Adipomes derived from T. cruzi-infected and TNFα-treated human adipocytes mostly ranged between 200 nm and 400 nm (FIG. 1D). The number of adipomes released from T. cruzi-infected and TNFα-treated adipocytes (in both murine and human adipocytes) were significantly higher (1.4-fold-2.8-fold) compared to the adipomes released from uninfected adipocytes. These findings indicated that T. cruzi infection leads to adipocyte apoptosis and that apoptotic adipocytes produce higher levels of adipomes than healthy adipocytes.

### Cultured Adipocyte-Derived Adipomes Regulate Adipogenic Signaling in Macrophages and Fibroblasts

We performed qPCR analysis of adipomes and found that adipomes contain adipogenic (Adipoq, Fabp4, Pparg), apoptotic (Chop), and extracellular vesicle (Cd13 and Tsg101) gene mRNAs (FIG. 1E). Thus, adipomes may influence adipogenic signaling in target cells, such as macrophages and fibroblasts. To test this hypothesis, first, we treated RAW 264.7 macrophages with adipomes obtained from murine 3T3-L1 cells at two different cell-to-adipome ratios (1:1 and 1:50). Second, we treated human foreskin fibroblast (HFF) cells with adipomes derived from cultured human adipocytes. Adipomes were isolated from uninfected adipocytes, T. cruzi-infected adipocytes, and adipocytes treated with TNFα (as a positive control for apoptosis).²³ Subsequently, we analyzed the gene expression in the target cells by qPCR analysis. Our findings have revealed that, in macrophages treated with adipomes derived from T. cruzi-infected adipocytes, the mRNA levels of adiponectin were significantly upregulated in the 1:50 treatment compared to naive macrophages (FIG. 1F). In HFF cells, adiponectin levels were significantly upregulated in both 1:1 and 1:50 adipome-treated cells compared to naive HFF cells. Additionally, this upregulation was observed in cells treated with adipomes from control or TNFα-treated adipocytes (FIG. 1F and FIG. 1G). Furthermore, to investigate the effects of upregulated adiponectin expression, we quantified the mRNA levels of adiponectin receptors (Adipor1 and Adipor2), Ppara, and Tnfa, all of which are regulated by adiponectin. Our observations showed that Ppara and Adipor2 levels were significantly upregulated in macrophages incubated with adipomes (1:50) from T. cruzi-infected and TNFα-treated adipocytes compared to naive macrophages and those treated with adipomes from control adipocytes (FIG. 10B). However, in HFF cells incubated with adipomes (1:50) from T. cruzi-infected adipocytes, the levels of Ppara and Adipor2 were significantly downregulated compared to untreated HFF cells and those treated with adipomes from control and TNFα-treated adipocytes (FIG. 10C).

Together, these experiments demonstrate that adipomes in general and adipomes from T. cruzi-infected adipocytes in particular upregulate adiponectin gene expression in macrophages and fibroblasts in a concentration-dependent manner. Additionally, the adipomes either have no effect or downregulate Tnfα in macrophages. Finally, adipomes differentially regulate the gene expression of Ppars, Adipor1, and Adipor2 in macrophages and HFF cells.

Acute T. cruzi Infection Promotes the Release of L-Adipomes in Mouse AT

Our in vitro data on the regulatory role of culture adipocyte-derived adipomes in macrophages and fibroblasts (FIG. 1F and FIG. 1G) suggested that adipomes derived from AT may play a critical role in regulating major myocardial cells during T. cruzi infection in vivo. To begin to address this question, we investigated whether adipocytes of infected mice release adipomes. In a previous study, we demonstrated that during acute infection with T. cruzi, there was an increase in the infiltration of immune cells and pro-inflammatory signaling in both white and brown AT.^{24,25} We also showed a significant loss of adipocytes during acute infection.^{17,26} When considering the production of pathological adipocytes and adipocyte-derived adipomes during T. cruzi infection, it is important to note that mice lack a true epicardial fat depot. Instead, they possess a small fat deposit around the heart,^{27,28} which is too small to isolate adipomes. Moreover, pericardial fat is rarely found in the acute Chagas mouse model, as we observed in the MRI images from our previous study, which showed the disappearance of pericardial fat during acute infection (FIG. 11A). Consequently, in this study, we chose to focus on visceral fat pads (epididymal white adipose tissue (WAT). First, we investigated whether T. cruzi infection induces apoptosis in WAT, leading to the loss of adipocytes and subsequent release of adipomes. Indeed, immunoblotting analysis of WAT revealed elevated levels of markers of lipolysis (phospho-Perilipin) and apoptosis (cleaved caspase 7 and Annexin V) in T. cruzi-infected mice compared to uninfected mice (FIG. 2A). These findings suggest that, as in cell culture, the T. cruzi infection of AT triggers adipocyte apoptosis and may lead to the release of apoptotic bodies and other macrovesicles.

It is well-known that large/macro EVs (large-EVs), such as ApoBDs and macrovesicles, differ in size and mechanism of origin from small or micro extracellular vesicles (small-EVs), such as microvesicles and exosomes.^{29,30,31,32,33} Therefore, in order to investigate the specific role of pathological adipocyte-derived adipomes in CD pathogenesis, we isolated and separated large- and small-EVs from WAT based on their size using high-speed centrifugation, as detailed in the STAR Methods. We observed that the levels of large-EVs were significantly higher in the WAT of infected mice (ranging between 2 and 10×10E7/100 mg) compared to uninfected mice (ranging between 5 and 10×10E5/100 mg), as quantitated by the Exocet exosome quantification kit (System Biosciences) with β-value ≤0.05. However, the levels of small-EVs did not show any significant difference between uninfected and infected mice, with counts ranging from 2.0 to 3.0×10E8. Additionally, immunoblotting analysis revealed the presence of the adipocyte-specific marker, adiponectin, in both large-EVs and small-EVs (FIG. 2B).

Developing an Innovative Method to Isolate Intact Adipomes from Mouse WAT

In the body, AT is comprised not only of adipocytes, but also various other cell types, including immune cells, that may contribute to the overall extracellular vesicle (EV) pool. Thus, to develop a method to specifically isolate adipomes derived from adipocytes, we investigated the suitability of adiponectin and FABP4, two widely used adipocyte markers, as surface markers that can be utilized for affinity purification. First, we investigated whether apoptosis regulates the expression of adiponectin and FABP4, and whether adiponectin and/or FABP4 are present on the cell surface of apoptotic adipocytes. We used murine 3T3L1 differentiated adipocytes (treated with TNFα for 24 h) and performed surface immunofluorescence analysis (IFA). Our observations revealed the co-localization of adiponectin and FABP4 with Annexin V (apoptosis marker) on the surface of budding apoptotic bodies (FIG. 2C and FIG. 11B). Annexins are present on the surface of EVs.^{34,35,36,37} Based on this in vitro data and previous studies demonstrating the presence of adiponectin and FABP4 in adipocyte-derived EVs,³⁸ we selected adiponectin and FABP4 as markers to isolate adipocyte-specific EVs/adipomes from the total pool of WAT-derived EVs.

Next, we successfully enriched intact L-adipomes (large-adipomes) and S-adipomes (small-adipomes) from large-EVs and small-EVs, respectively, following the procedure described (FIG. 10). Immunoblotting analysis confirmed the presence of adiponectin, FABP4, Annexin V, and perilipin in both L- and S-adipomes, providing further evidence of their adipocyte origin (FIG. 2D and FIG. 11C). Moreover, surface immunostaining of bead-bound adipomes provided additional support for the use of adiponectin and FABP4 as suitable markers for identifying and isolating adipomes from a pool of EVs (FIG. 2E). Fluorescence microscopic analysis revealed a stronger green fluorescence intensity in adipomes stained for surface adiponectin compared to FABP4-stained adipomes, indicating that adiponectin is more abundant on the surface of adipomes compared to FABP4 (FIG. 2E). As a negative control, we also stained the magnetic streptavidin beads to check for autofluorescence or non-specific antibody binding and found no fluorescence signal (FIG. 11D). Further characterizations of adipomes included determining their size distribution and abundance using Spectradyne nCS1 (FIG. 2F) and examining their size and integrity through TEM analysis (FIG. 2G and FIG. 11E, FIG. 11F, and FIG. 11G). Spectradyne measurements produced distinct peaks for L- and S-adipomes (FIG. 2F), while TEM images of L-adipomes exhibited the presence of apoptotic budding scars, with a size range between 200 and 400 nm. In contrast, the size of S-adipomes ranged between 30 and 80 nm, as shown by TEM images (FIG. 2G and FIG. 11E, FIG. 11F, FIG. 11G). These findings validate our innovative method for isolating adipocyte-specific adipomes from WAT.

L-Adipomes and S-Adipomes Carry a Distinct Set of Lipid Cargoes, which Differ Between Uninfected and Infected Mice

Adipocytes in WAT contain large amounts of lipids compared to other cells.³⁹ During infection, the presence of parasites may alter adipocyte lipid composition. Thus, adipomes may also carry a substantial amount of lipid biomolecules, and their lipid contents may vary between infected and uninfected mice. To investigate whether the lipid cargoes differ between large adipomes (L-adipomes) and small adipocytes (S-adipomes) and between pathological and normal conditions (infection vs. non-infection), we conducted Liquid Chromatography-Mass Spectrometry-based lipidomics analysis of L-adipomes and S-adipomes derived from WAT of control (uninfected) mice (CLA and CSA, respectively) and infected mice (ILA and ISA, respectively). A total of 287 lipid metabolites were identified, covering more than 21 subclasses including free cholesterol, free fatty acids (FFA), sphingomyelins (SM), phospholipids, ceramides (CER), triglycerides (TAG), and so forth. These lipids were quantified using class-specific internal standards (see STAR Methods). The lipidomic analysis of CLA, ILA, CSA, and ISA revealed diverse lipid patterns specific to the pathological conditions and the two types of adipomes (large vs. small) (FIG. 3 and FIG. 12). For example, cholesterol levels were higher in ILA and ISA compared to CLA and CSA, while total FFA levels were lower in ILA and ISA compared to CLA and CSA. Additionally, total diacylglycerol (DAG) and TAG levels were greater in ISA compared to all other groups (FIG. 12A). ILA contained higher levels of total ceramides, hydroxy ceramides (HCER), SM, and various phospholipids compared to CLA (FIG. 12A).

Even in cases where specific lipid classes were not significantly altered between the groups, the levels of many individual lipid metabolites within each class were differentially expressed (DE) and specific to CLA, CSA, ILA, and ISA (the complete list of DE lipid metabolites in CLA, CSA, ILA, and ISA are available in Table S1 (see appendix). Further analysis comparing CLA and ILA showed significant differences in several lipid metabolites between these two groups (FIG. 3A, FIG. 3B, FIG. 3C). We applied Principal component analysis (PCA) to identify the metabolic profile differences between groups (FIG. 3A). First, a heatmap was used to find the relative intensity distribution of the significantly altered lipid metabolites and to identify differentially expressed lipid metabolites between CLA and ILA, as shown in FIG. 3B. We next constructed a volcano plot applying a significance β-value level of <0.05 based on t-test and a fold change (FC) value higher than 1.5 (FIG. 3C). The volcano plot analysis revealed 15 lipid molecular species significantly altered in ILA relative to the CLA group. Notably, levels of PC(18:1/16:1), PE(18:0/20:4), PE(P-16:0/22:6), HCER(16:0), HCER(26:1), SM(d18:1/22:0), SM(d18:1/22:6), SM(d18:1/17:0), SM(d18:1/16:0), CER(18:0), pentadecanoyl carnitine-AC (15:0), tetradecanoyl AC (14:0), TAG48:3-FA18:2, and TAG52:2-FA18:1 were significantly increased, while levels of PE(β-18:2/18:2) were significantly decreased in ILA compared to CLA (FIG. 3C).

To identify the molecular mechanisms associated with the lipid metabolites enriched in ILA compared to CLA, we employed the Pathway Topology Analysis (PTA) module. The PTA module combines powerful pathway enrichment analysis with pathway topology analysis to identify the most relevant pathways altered by the metabolites (see STAR Methods). Pathway Analysis of the upregulated lipid metabolites in ILA compared to CLA revealed that the enriched lipid metabolites in ILA are involved in seven different lipid signaling pathways, including Sphingolipid metabolism, Glycerophospholipid metabolism, Linoleic acid metabolism, alpha-Linolenic acid metabolism, Glycosylphosphatidylinositol (GPI)-anchor biosynthesis, Glycerolipid metabolism, and Arachidonic acid metabolism (Table 1; FIG. 3D).

TABLE 1
Upregulated metabolites in ILA compared to CLA and their
respective KEGG metabolite ID (Related to FIG. 3).

| Metabolites | KEGG |
|---|---|
| PC(14:0/20:2) | C00157 |
| Ceramide (d18:1/18:0) | C00195 |
| TG(16.0/18:0/18:2) | C00422 |
| PE(0-16:1/22:6) | C00350 |
| Galactosylceramide (d18:1/16:0) | C00350 |
| Galactosylceramide (d18:1/26:1) | C02686 |
| SM(d18:1/22:0) | C00550 |
| SM(d18:0/16:1(OH)) | C00550 |
| SM(d18:0/16:1) | C00550 |

Based on the KEGG numbers of lipid metabolites, we identified five metabolites involved in Sphingolipid metabolism (such as SM(d18:1/22:0) [KEGG #C00550], SM(d18:0/16:1 [KEGG #C00550], SM(d18:0/16:1 [KEGG #C00550]), Galactosylceramide (d18:1/16:0) [KEGG #Cβ2686], and Galactosylceramide (d18:1/26:1) [KEGG #Cβ2686]), three metabolites in Glycerophospholipid signaling (such as PC(14:0/20:2 [KEGG #C00157]), PE(O-16:1/22:6) [KEGG #C00350], and PE(16:0/22:4) [KEGG #C00350]), and one metabolite in Linoleic acid pathway PC(14:0/20:2 [KEGG #C00157]) (FIG. 3E and FIG. 3F; Table S2). Impaired accumulation of these metabolites and associated pathways has been shown to increase the risk of cardiovascular and coronary artery diseases.^{40, 41} Thus, these analyses suggest that the interaction of cardiomyocytes with ILA may impair cardiomyocyte functions through alterations in sphingolipid, glycerophospholipid, and linoleic acid metabolisms.

TABLE S2
Enriched pathways associated with upregulated lipid metabolites
in ILA compared to CLA, Related to FIG. 3 and Table 1.

| | | | | | Holm | |
|---|---|---|---|---|---|---|
| Pathway | Total | Hits | Raw p | -log10(p) | Adjust | FDR |
| Sphingolipid metabolism | 21 | 3 | 4.18E−05 | 4.38E+00 | 3.51E−03 | 3.51E−03 |
| Glycerophospholipid | 36 | 2 | 7.42E−03 | 2.13E+00 | 6.16E−01 | 3.12E−01 |
| metabolism | | | | | | |
| Linoleic acid metabolism | 5 | 1 | 1.92E−02 | 1.72E+00 | 1.00E+00 | 5.38E−01 |
| Alpha-Linolenic acid | 13 | 1 | 4.94E−02 | 1.31E+00 | 1.00E+00 | 8.46E−01 |
| metabolism | | | | | | |
| Glycosylphosphatidylinositol | 14 | 1 | 5.31E−02 | 1.28+00 | 1.00E+00 | 8.46E−01 |
| ancho biosynthesis (GPI) | | | | | | |
| Glycerolipid metabolism | 16 | 1 | 6.05E−02 | 1.22E+00 | 1.00E+00 | 8.46E−01 |
| Arachidonic metabolism | 36 | 1 | 1.32E−01 | 8.80E−01 | 1.00E+00 | 1.00E+00 |

We also compared lipid metabolite levels between ILA and ISA to identify any differences in lipid contents between the large and small adipomes derived from infected mice (FIG. 3G, FIG. 3H, FIG. 3I). The PCA revealed a clear separation between the two groups (ILA and ISA) (FIG. 3G), and we observed differentially expressed metabolites between ILA and ISA samples (FIG. 3H). Specifically, the levels of various TAGs (more than 50) were significantly decreased in ILA compared to ISA, with the exception of LPE (20:0), which was significantly higher in ILA (FIG. 3I). Similarly, we observed differences in DE lipid metabolites between ISA and CSA (FIG. 12B and FIG. 12C). Notably, the levels of various TAGs were significantly higher in ISA compared to CSA (FIG. 12C). This data further supports the idea that ISAs are rich in TAGs, which may be important in how they regulate cellular functions. Furthermore, we analyzed whether lipid profiles differ between CLA and CSA and found that CSA carry significantly greater levels of various SMs, CERs, and HCERs compared to CLA (FIG. 12D and FIG. 12E). Overall, our data indicates that CLA, ILA, CSA, and ISA carry different lipid cargoes specific to their origin.

Adipomes Derived from AT of T. cruzi-Infected and Uninfected Mice Differently Regulate Immunometabolic Signaling in Macrophages and Alter Macrophage Polarization

We have demonstrated a significant regulatory impact of cultured adipocyte-derived adipomes on gene expression in macrophages (FIG. 1F). Additionally, our research has revealed that WAT-derived adipomes carry specific lipid cargoes unique to infection status and adipome type (FIG. 3). These findings suggest that adipomes derived from uninfected and infected mice may differentially influence macrophages, which play a vital role in pathogen and dead cell clearance. For instance, adipomes enriched with ceramides (ILA) can induce macrophage M1 polarization,⁴² while those enriched with triacylglycerols (ISA) may cause macrophage mitochondrial oxidative stress.⁴¹ Thus, we explored the regulatory effects of WAT-derived adipomes in macrophages ex vivo. To assess the impacts of adipomes from different sources (CLA, ILA, CSA, and ISA) on macrophages, we treated RAW 264.7 macrophages with adipomes at a 1:1 cell-to-adipome ratio for 48 h. Subsequently, we performed qPCR analysis of both treated and untreated macrophages using a custom-designed RT2 Profiler qPCR array (Qiagen). This array includes genes associated with adipogenesis (Adipoq, Pparg, Fabp4), lipogenesis (Cebpa, Srebf1, Fas), cellular metabolism-specific signaling molecules (Slc2a4, Sphk1, Sgk2, Cpt1b, Slc27a4, Adra1a, Adra1b, Adipor1, Adipor2, and Insr), as well as markers of inflammatory and immune responses (Adgre1, Ccl5, Cxcl10, Ifng, Il1a, Il1b, Il10, Il17a, 112, Il4, Il6, Tnfa, Tlr2, Tlr4, Tlr9, Ccr5, Ccr6, Cd80, Cxcr3, Cxcr5, Foxp3, Cd40, Cd86, Myd88, and Nfkb1) (FIG. 13A).

First, we compared the gene expression between macrophages treated with L-adipomes (CLA or ILA-treated groups) and naive macrophages. We observed a significant upregulation of Adipoq (adiponectin) and Pparg in macrophages treated with CLA or ILA compared to naive cells (FIG. 4A). Additionally, with the exception of Cebpa, Srebf1, Fabp4, Slc27a4, adiponectin receptors Adipor1 and Adipor2, and Insr, all other genes involved in metabolic signaling exhibited significant increases in macrophages treated with CLA/ILA compared to naive cells (FIG. 4B). Moreover, the mRNA levels of pro-inflammatory signaling molecules such as Tnfα, Tlr2, Tlr4, Tlr9, and Myd88 were significantly decreased, while the levels of anti-inflammatory signaling molecule 1110 were significantly increased in macrophages treated with CLA/ILA compared to naive macrophages (FIG. 4C). Furthermore, qPCR data demonstrated significantly higher levels of Ifng, 116, and Ccr5 in macrophages treated with CLA/ILA (FIG. 4D). It is noteworthy that even though the levels of Ifng and 116 were significantly upregulated in adipome-treated macrophages, the pro-inflammatory cytokine Tnfα was significantly downregulated compared to naive macrophages, suggesting a possible shift in macrophage polarization toward M2.

As part of our investigation into macrophage polarization, we examined the mRNA levels of Arginase (Arg1), which serves as a marker of M2 macrophages and plays a key role in macrophage activation and polarization. The results revealed a significant increase in Arg1 levels in macrophages treated with adipomes compared to naive macrophages (FIG. 14A). Moreover, when comparing CLA and ILA treated cells, we observed significantly higher levels of Arg1 in the ILA-treated cells (FIG. 4E). Interestingly, both CSA and ISA-treated macrophages showed significantly reduced levels of Arg1 compared to CLA and ILA-treated cells, respectively (FIG. 14B). These findings indicate that adipomes regulate metabolic and immune signaling in macrophages by inducing adiponectin expression, inhibiting Nfkb-Tnfα activation, and promoting M2 polarization. Additionally, these data suggest that ILA significantly upregulates adipogenic genes, I110, and Arg1 in macrophages compared to CLA, CSA, and ISA. This finding may have implications for macrophage activation in infected mice and the clearance of parasites from infected organs.

Quantitative analysis revealed significant differences between macrophages treated with adipomes and untreated macrophages (FIG. 13B). The genes most significantly and highly upregulated in adipome-treated macrophages compared to naive macrophages were Adipoq, Pparg, Ifng, I16, and Ccr5 and downregulated were Nfkb, Tnfa, Cebpa, Srebp1, Adipor1, Adipor2 and Insr, indicating that adipomes regulate immunometabolic genes in macrophages. Furthermore, the origin of adipomes (CLA, WLA, CSA, and ISA) was also associated with notable differences in the levels of specific genes (Tables S3 and S4). For example, in the comparison between ISA- and CSA-treated macrophages, the mRNA levels of I14, and Ccr5 (anti-inflammatory signaling) were significantly downregulated in ISA-treated macrophages compared to CSA-treated macrophages, suggesting that infection generated ISA promotes pro-inflammatory signaling.

TABLE S3
Differentially altered genes between ILA/ISA in adipome-
treated macrophages. Related to FIG. 4, FIG. 13 and FIG. 14

| Gene List | Up- or Down-Regulation | p-value |
|---|---|---|
| Adipoq | 4.57 | 0.000706 |
| Lep | 4.01 | 0.000003 |
| Cebpa | 3.31 | 0.000009 |
| Srebf1 | 3.30 | 0.001112 |
| Fabp4 | 3.56 | 0.010842 |
| Fas | 4.26 | 0.000741 |
| Pparg | 4.96 | 0.002728 |
| Ppara | 4.55 | 0.000005 |
| Adgre1 | 3.93 | 0.000080 |
| Slc2a4 | 3.93 | 0.000006 |
| Sphk1 | 4.70 | 0.000654 |
| Sgk2 | 4.78 | 0.000004 |
| Cpt1B | 3.54 | 0.000003 |
| Slc27a4 | 3.34 | 0.001548 |
| Adra1a | 4.12 | 0.000001 |
| Adra1b | 4.51 | 0.000006 |
| Adipor1 | 3.51 | 0.004089 |
| Adipor2 | 3.70 | 0.000007 |
| Insr | 2.85 | 0.000026 |
| Ccl5 | 4.78 | 0.007528 |
| Cxcl10 | 3.28 | 0.000006 |
| Ifng | 4.73 | 0.000002 |
| Il1a | 3.69 | 0.035066 |
| Il1b | 4.29 | 0.000002 |
| Il10 | 4.76 | 0.000001 |
| Il17a | 3.78 | 0.000891 |
| Il2 | 4.36 | 0.000004 |
| Il4 | 4.29 | 0.000003 |
| Il6 | 4.84 | 0.007568 |
| Tnf | 3.11 | 0.000012 |
| Tlr2 | 3.16 | 0.000011 |
| Tlr4 | 2.82 | 0.006077 |
| Tlr9 | 3.20 | 0.000005 |
| Ccr5 | 4.39 | 0.000001 |
| Ccr6 | 4.62 | 0.00648 |
| Cd80 | 3.18 | 0.000015 |
| Cxcr3 | 3.73 | 0.000002 |
| Cxcr5 | 3.53 | 0.000007 |
| Foxp3 | 3.49 | 0.001036 |
| CD40 | 2.87 | 0.000024 |
| CD86 | 4.19 | 0.000002 |
| Myd88 | 3.54 | 0.001027 |
| Cd4 | 4.35 | 0.000007 |
| Cd8a | 3.87 | 0.000873 |

The upregulation of adipogenic genes, such as Adipoq and Pparg, and the downregulation of other genes involved in glucose and lipid metabolism, such as Cebpa, Srebf1, and Slc27a4, in adipome-treated macrophages could potentially impact energy pathways and differentially influence mitochondrial functions relative to naive macrophages. To explore this further, we analyzed the mRNA levels of mitochondrial genes associated with oxidative phosphorylation, including Nadhd, Nd1, Nd2, Sdhc, Cytb, Cox1a, Cox5a, Apt6, Ant1, and Ppargc1a. Our qPCR analysis revealed a significant increase in Nadhd, Nd1, Nd2, Cytb, Cox1a, and Apt6 in all the adipome-treated groups compared to naive macrophages (FIG. 14C). Furthermore, when we compared the levels of these genes between CLA- and ILA-treated cells, we found that all these genes were significantly increased in ILA-treated macrophages compared to CLA-treated macrophages (FIG. 4F). Additionally, the levels of Cox5a and Ant1 were significantly decreased in CSA- and ISA-treated macrophages compared to naive macrophages (FIG. 4F). Moreover, between ILA- and ISA-treated macrophages, the above-mentioned genes were significantly upregulated in macrophages treated with ILA compared to ISA, suggesting that ILA may highly regulate mitochondrial functioning genes. These findings indicate significant alterations in mitochondrial gene expression among macrophages treated with adipomes of different origin (FIG. 4F and FIG. 14D, FIG. 14E, FIG. 14F). These results suggest that, depending on the state of AT (e.g., infected vs. non-infected), AT-derived adipomes can influence mitochondrial gene expression differently in macrophages, potentially impacting their cellular energy metabolism and functions.

### Adipomes Upregulate Lipogenic, Mitochondrial and Inflammatory Genes in Cardiomyocytes

To investigate whether adipomes can also regulate non-phagocytic cells in the heart, we treated murine primary cardiomyocytes with adipomes (1:1) derived from different sources (CLA, ILA, CSA, and ISA) for 48 h. Subsequently, we analyzed the mRNA expression of various genes, including Adipoq and adiponectin receptors (Adipor1 and Adipor2), lipolytic signaling (β-AR, beta-Adrenergic Receptor, Adrb2), lipogenic genes (Srebp1a and Srebp1c), mitochondrial signaling (Nadhd, Nd1, Nd2, Sdhc, Cytb, Cox1a, Cox5a, Apt6, Ant1, and Ppargc1a), and inflammatory genes (Tnfa and Ifng) by qPCR.

qPCR analysis revealed that adipomes from all the groups significantly upregulated the expression of Adrb2, Tnfa, Srebpla, and Nd2 in cardiomyocytes compared to untreated cells (FIG. 5A, FIG. 5B, FIG. 5C). However, ILA treatment significantly altered most of the genes tested, with the exception of Adipoq, Srebp2, Sdhc, and Cytb (FIG. 5A, FIG. 5B, FIG. 5C). In the comparison between ILA- and CLA-treated macrophages, the levels of Pparg were significantly upregulated in ILA-treated cells compared to CLA-treated cells (Figure S6A). qPCR demonstrated a substantial upregulation of adiponectin receptors (Adipor1 and Adipor2) in adipome-treated cardiomyocytes, while the levels of adiponectin remained unchanged compared to untreated cardiomyocytes (FIG. 5A). Specifically, the levels of Adipor1 and Adipor2 were significantly higher in cardiomyocytes treated with ILA and CSA compared to those treated with CLA. Adipome treatment also led to a significant increase in the expression of Adrb2 and Srebp1a genes in cardiomyocytes, suggesting that adipomes alter lipid metabolism. Furthermore, Srebp1c was significantly increased in ILA and not in CLA compared to untreated cardiomyocytes.

This increased lipogenic signaling and stimulation of lipolysis through j-AR appears to upregulate mitochondrial functions.^{44,45} Among the analyzed genes encoding mitochondrial proteins, adipome treatment (regardless of origin) significantly altered the levels of Nd2, Cox1a, and Cox5a genes in cardiomyocytes (FIG. 5B). The levels of the Nd1 gene were significantly upregulated in cells treated with ILA and CSA compared to untreated cells. Increased mitochondrial oxidative phosphorylation leads to an increase in reactive oxygen species production, which in turn influences TNFa in a positive feedback loop.⁴⁶ Indeed, our data also showed a significant increase in the mRNA levels of Tnfα in adipome-treated cardiomyocytes compared to untreated cells (FIG. 5C). Additionally, qPCR demonstrated an upregulation of Ifng levels in ILA-, CSA-, and ISA-treated cells, but not in CLA-treated cells, compared to untreated cardiomyocytes (FIG. 5C). These findings suggest that adipomes enhance lipogenic signaling in cardiomyocytes, leading to increased mitochondrial oxidative phosphorylation and inflammatory signaling. Notably, ILA treatment further upregulated lipogenic signaling compared to CLA via the upregulation of Pparg (FIG. 15A). No significant difference in macrophage gene regulation was observed between ILA and ISA-treated macrophages, whereas CSA treatment significantly increased lipogenic signaling and mitochondrial oxidative signaling compared to CLA, suggesting that uninfected WAT-derived L- and S-adipomes differently regulate macrophages. Overall, our data demonstrate that CLA, ILA, CSA, and ISA regulate both macrophages and cardiomyocytes, and that they differentially alter adipogenic, lipogenic, mitochondrial oxidative, and inflammatory signaling in these target cells.

### Plasma-Derived Infection-Associated Adipomes (L- and S-) Induce Endoplasmic Reticulum Stress in Primary Cardiomyocytes

Our qPCR analysis revealed that adipomes derived from AT regulate the mRNA expression of β-AR, a marker of arrhythmic cardiomyopathy (FIG. 5). Thus, we also analyzed the protein levels of β-AR in cardiomyocytes exposed to adipomes (at a cell-to-adipome ratio of 1:25 for 48 h) by immunoblotting analysis. Notably, the levels of β-AR were significantly higher in cardiomyocytes treated with plasma-derived L-adipomes isolated from infected mice compared to naive cardiomyocytes (FIG. 6 and FIG. 10A). β-AR stimulation is known to induce endoplasmic reticulum (ER) stress in cardiomyocytes.47 Consequently, we analyzed the protein levels of CHOP, an ER stress marker, and discovered that both L- and S-adipomes specifically from infected mice plasma significantly increased the levels of CHOP in cardiomyocytes compared to naive cells. This data indicates that plasma-derived L-adipomes from infected mice regulate j-AR signaling, which is linked to lipid metabolism and arrhythmias in cardiac cells.

### P-ILA Induce Mitochondrial Dysfunction in Primary Cardiomyocytes

Increased lipogenic signaling can strain mitochondrial functions, potentially leading to mitochondrial dysfunction and arrhythmias.^{48,49,50} To investigate whether PILA-induced arrhythmogenic j-AR signaling in cardiomyocytes is attributable to heightened lipogenic signaling-associated mitochondrial dysfunction, we assessed mitochondrial bioenergetics by Seahorse XFe96 analyzer, using the Seahorse XF Cell Mito Stress Test kit as described in the STAR Methods section. We measured changes in oxygen consumption rate (OCR) in P-ILA-treated mouse primary cardiomyocytes, revealing significantly reduced basal and maximal respiration, as well as ATP production, in P-ILA-treated cardiomyocytes compared to both naive and P-CLA-treated cells (FIG. 7). These data suggest that P-ILA induces mitochondrial dysfunction and exacerbates arrhythmias.

### P-ILA Elevate the Risk for Cardiomyopathy in Wild-Type Mice

We previously demonstrated that inducing adipocyte apoptosis increases the risk of cardiomyopathy in T. cruzi-infected mice during both the acute and chronic stages of infection.¹¹ In this study, we further showed that AT from infected mice releases higher levels of large adipomes (L-adipomes), and their lipid contents differ compared to those of uninfected mice. To investigate whether L-adipomes exist in circulation and can regulate the myocardium during infection, we isolated L-adipomes from the plasma of T. cruzi-infected (20 DPI) and wild-type C57BL/6J mice. Plasma-derived infection-associated large adipomes (P-ILA) from infected mice displayed a size range of 300-800 nm, as analyzed by Spectradyne (FIG. 10A and FIG. 16A). Ultrasound-guided intramyocardial adipome injection was performed twice a week, with PBS-injected wild-type C57BL/6J mice serving as controls. Cardiac ultrasound analysis one week after the initial treatment revealed significant alterations in cardiac morphology in mice treated with P-ILA compared to the control group, including an increased left-ventricle internal diameter (LVID), right-ventricle internal diameter (RVID), and septal wall thickness at diastole (d) (Table 2). Mice treated with P-ILA also exhibited a greater left-ventricle ejection fraction (EF) compared to mice treated with the vehicle (64.383±4.040 vs. 42.817±3.325; Table 2). It is important to note that these measurements were carried out in sedated mice, and, as reported previously,⁵¹ sedation reduced the normal EF level from approximately 65% to approximately 35% in C57BL/6J mice. Notably, increased EF levels have been observed in murine models of cardiac hypertrophy⁵² and according to the American Heart Association, an EF measurement higher than 75% could indicate a heart condition such as hypertrophic cardiomyopathy in patients.⁵³ Thus, the significantly increased EF levels in P-ILA-treated mice compared to vehicle-treated mice suggest that P-ILA treatment may induce a hypertrophic cardiomyopathy phenotype in wild-type C57BL/6J mice.

TABLE 2
Cardiac ultrasound analysis (M mode) showing altered
LVID, RVID and other parameters including EF in mice
treated with P-ILA compared to control mice.

| ECG* Measurement | Vehicle | P-ILA |
|---|---|---|
| LVID (d) mm | 3.695 ± 0.083 | 3.999 ± 0.095** |
| LVID(s) mm | 2.643 ± 0.057 | 2.675 ± 0.125 |
| LVPW (d) mm | 4.950 ± 0.233 | 5.330 ± 0.070* |
| LVPW (s) mm | 4.055 ± 0.385 | 4.265 ± 0.095 |
| RVID (d) mm | 0.976 ± 0.142 | 1.462 ± 0.002** |
| RVID (s) mm | 0.638 ± 0.087 | 0.764 ± 0.024 |
| RVED (d) mm | 1.503 ± 0.181 | 1.932 ± 0.059* |
| RVED (s) mm | 1.275 ± 0.094 | 1.492 ± 0.052* |
| Septal Wall (d) mm | 0.471 ± 0.018 | 0.599 ± 0.016*** |
| Septal Wall (s) mm | 0.325 ± 0.006 | 0.398 ± 0.010*** |
| LVEF | 42.817 ± 3.325 | 64.383 ± 4.040** |

*ECG, electrocardiography; LVID, left ventricular internal diameter; LVPW, left ventricular posterior wall; RVID, right ventricular internal diameter; RVED, right ventricular end-diastolic area; LVEF, left ventricular ejection fraction; d, diastole; s, systole; P-ILA, plasma-derived infection-associated L-adipomes.
*p ≤ 0.05,
**p ≤ 0.01 compared to control mice (vehicle-treated).

### Alteration of Inflammatory and Metabolic Signaling in the Heart by P-ILA Increases Cardiomyopathy Risk in Post-Acute Chagas Mouse Model

The data presented above suggested that P-ILA treatment may exacerbate cardiac pathology in CD mice. To investigate whether P-ILA affect cardiac pathology and whether they may exacerbate the cardiomyopathic phenotype during chronic infection, we replicated the in vivo experiment using both uninfected and post-acute T. cruzi-infected (35 DPI) C57BL/6J mice. Both uninfected and T. cruzi-infected mice were treated with PBS to serve as appropriate controls. Ten days after the initial treatment, the mice were sacrificed, and their hearts were harvested for histological and biochemical analysis to assess the impact of adipome treatment on the myocardium.

The H&E sections of the hearts revealed an increased infiltration of immune cells in the hearts of both P-ILA-treated uninfected and infected mice, specifically in the epicardium and right ventricles (FIG. 16B and FIG. 16C). An analysis assessing the impact of adipome treatment on fibrosis showed no significant differences in fibrosis levels between mice that received adipome treatment and those that did not, in both uninfected and infected groups (FIG. 16D). However, fibrosis levels notably increased in the infected groups (both P-ILA-treated and untreated) compared to uninfected groups. This was evident in the results of Picrosirius Red Polarization (PSRP) staining (FIG. 16D). Considering the regulatory influence of adipomes on the expression of TNFA in cultured macrophages and primary cardiomyocytes in vitro (FIG. 4 and FIG. 5), we also investigated their effect on TNFA protein expression in the myocardium through immunohistochemical (IHC) analysis. We found that treatment with P-ILA elevated the levels of TNFA in the myocardium compared to treatment with the vehicle in uninfected mice but not in infected mice. However, the levels of TNFA were significantly increased in the myocardium of infected mice compared to uninfected mice (FIG. 16E). We further investigated the protein levels of inflammatory markers, namely TNFA, IFNG, and IL6, in the heart lysates through immunoblotting analysis. The analysis revealed significantly higher levels of TNFA and IL6 in P-ILA-treated uninfected mice and only IL6 in P-ILA-treated-infected mice compared to their respective vehicle-treated groups (FIG. 8A). Immunoblotting analysis of F4/80 in heart lysates demonstrated that P-ILA treatment also significantly increased the infiltration of macrophages into the hearts (FIG. 8A). Together, these data suggest that P-ILA treatment induces macrophage infiltration in the hearts of both uninfected and infected mice and increases TNFA only in uninfected mice but does not alter M1-cytokine markers such as TNFA and IFNG in infected mice.

In addition to its key role during inflammation, the pleiotropic cytokine IL6 is involved in lipid metabolism in both humans and rodents by stimulating lipid hydrolysis and mitochondrial β-oxidation to prevent cardiac lipotoxicity.^{54,55} Therefore, we analyzed the levels of SREBP1 and perilipin, markers of lipogenesis and lipid droplets, respectively, and found that the levels of both these proteins significantly increased in the hearts of P-ILA-treated uninfected mice but not in infected mice compared to their respective vehicle-treated groups (FIG. 8B). This data mirrors our in vitro observations where adipome treatment significantly increased lipogenic genes in primary uninfected cardiomyocytes (FIG. 8B). Interestingly, we noted that the levels of both SREBP1 and perilipin were significantly decreased in vehicle-treated infected mice compared to vehicle-treated uninfected mice, indicating that acute infection reduces lipid levels in the hearts, either inhibiting lipogenesis or by increasing lipid utilization in the heart. To test whether P-ILA treatment increases cardiac lipid hydrolysis and oxidation, we analyzed the levels of phospho-perilipin (p-Perilipin) and PPARα, markers of lipid hydrolysis and lipid oxidation, respectively.^{56,57} We found that the levels of p-Perilipin and PPARα significantly increased in the hearts of infected mice compared to uninfected groups, and the treatment with β-ILA further significantly increased the levels of both markers in infected mice (FIG. 8B). These data suggest that increased lipid hydrolysis may elevate mitochondrial β-oxidation.⁵⁸

Our in vitro data indicated that adipomes regulate mitochondrial genes involved in oxidative phosphorylation. To further explore these findings in vivo, we analyzed the levels of markers of mitochondrial oxidative phosphorylation, such as SDHA, Cytochrome c, COX IV, and HSP60 (FIG. 8C). P-ILA treatment significantly increased the levels of SDHA and HSP60 only in uninfected mice (but not in infected mice) compared to vehicle treatment (FIG. 8C). Interestingly, P-ILA treatment significantly reduced the levels of cytochrome c in both uninfected and infected mice and significantly reduced COX IV only in the infected mice compared to vehicle treatment. These data suggest that P-ILA treatment affects mitochondrial oxidative phosphorylation and may have especially deleterious effects on the hearts of infected mice, where lipid hydrolysis is significantly increased compared to uninfected mice.

### P-ILA Induces ER Stress in the Heart, Elevating Cardiomyopathy Risk in Post-Acute Chagas Mice

Previously, we demonstrated that the onset of cardiomyopathy in T. cruzi-infected mice, induced by adipocyte apoptosis, is associated with decreased mitochondrial oxidation (cytochrome c levels) and increased ER stress.¹¹ Therefore, we analyzed the levels of ER stress markers—Binding immunoglobulin protein (BiP), protein kinase R-(eIF2α), protein disulfide isomerase (PDI) and C/EBP homologous protein (CHOP)—in heart lysates. Our findings indicate a significant increase in the levels of BiP, PDI, and phospho-eIF2α in the hearts of P-ILA-treated-infected mice and only BiP in the hearts of P-ILA-treated uninfected mice compared to the respective vehicle-treated groups (FIG. 8D). Elevated BiP and phospho-eIF2α levels are indicative of increased ER stress.⁵⁹ ER stress, in turn, is known to raise CHOP levels, which can activate apoptosis.^{60,61} Indeed, immunoblotting analysis data revealed an increase in both CHOP and cleaved caspase (an apoptosis marker), but not BNIP3 (a necrosis marker), in P-ILA-treated-infected mice (FIG. 8D and FIG. 8E), indicating that P-ILA induces ER stress and apoptosis while concurrently diminishing mitochondrial functions in the hearts in infected mice. However, in uninfected mice, although P-ILA treatment increased BiP and CHOP levels (but not phospho-eIF2α), the levels of cleaved caspase were not increased compared to vehicle treatment. This could be due to increased HSP60 levels in the hearts of P-ILA-treated uninfected mice, which can promote cell survival and inflammation as indicated by increased TNFα levels in the hearts.^{62,63} Our data indicates that in uninfected mice, P-ILA treatment induces ER stress associated with lipogenesis, impaired mitochondrial oxidative phosphorylation, and elevated proinflammatory TNFα signaling, whereas in infected mice, P-ILA treatment induces ER stress associated with lipid hydrolysis, impaired mitochondrial oxidative phosphorylation, and elevated apoptosis signaling.

Next, drawing on previous reports,^{5,1164,65} we analyzed specific markers of cardiomyopathy using heart lysates and heart sections to further clarify the role of P-ILA in regulating CCM. Chagas heart disease is considered an arrhythmogenic cardiomyopathy characterized by atrial and ventricular arrhythmias and a wide variety of abnormalities of the conduction system.¹¹ Beta-adrenergic receptors in the heart play a crucial role in regulating heart rate and contractility,⁶⁶ and overactivation of beta-adrenergic receptors can potentially lead to arrhythmias.⁶⁷ CD is also known to increase the levels of β1-AR and ANP in the hearts of experimental animals and patients.^{64,65} Murine CD models with cardiomyopathy display elevated levels of adiponectin in the hearts.¹¹ Immunoblotting analysis of heart lysates demonstrated that P-ILA treatment significantly increases the levels of adiponectin and β1-AR in the hearts of mice compared to vehicle treatment, both in infected and uninfected mice (FIG. 9A). IHC analysis further revealed elevated ANP levels in the hearts of P-ILA-treated mice compared to vehicle-treated mice (FIG. 9B), suggesting that P-ILA treatment induces cardiomyopathy in uninfected mice and exacerbates the severity of cardiomyopathy in post-Chagas-infected mice.

P-ILA do not Carry T. cruzi in their Cargo

To investigate whether the observed effects in the hearts of P-ILA-treated mice were due to P-ILA-associated T. cruzi, we conducted qPCR analysis of T. cruzi-specific gene in heart extracts, as previously demonstrated.¹⁷ Our results revealed no trace of parasites in the hearts of both P-ILA-treated and untreated post-infected mice, and no detectable parasites in β-ILA-treated uninfected mice (FIG. 17). The near absence of parasite genetic material in post-infected mice (45 DPI) can be attributed to the use of a low parasite load (10E3) in the initial infection, which typically diminishes significantly in the hearts following acute infection.¹⁷ The lack of a significant increase in parasite genetic material in P-ILA-treated post-infected and uninfected mice suggests that adipomes do not harbor parasites. To reinforce this finding, we conducted qPCR analysis of T. cruzi in P-ILA-treated cultured cardiomyocytes (1:20 ratio, 48 h), revealing the absence of parasites in treated cells (FIG. 17). Furthermore, the size of the parasite ranges between 2 and 6 m, which is significantly larger than that of L-adipomes (FIG. 16A), making it unlikely that adipomes harbor parasites.

Overall, our in vivo data demonstrate that intracardiac P-ILA treatment increases the infiltration of macrophages, elevates β-AR, adipogenic and IL6 signaling, impairs mitochondrial oxidative phosphorylation, and induces ER stress in the myocardium. In addition, P-ILA regulate lipogenesis and TNFα signaling in the hearts of uninfected mice and lipid hydrolysis and apoptosis signaling in infected mice, leading to hypertrophied cardiomyopathy and exacerbating cardiac pathology in uninfected and post-acute CD mice, respectively.

### DISCUSSION

Chagas cardiomyopathy, which typically develops after several years or decades in approximately 30% of T. cruzi-infected patients, manifests a broad spectrum of cardiac abnormalities, ranging from ECG abnormalities to multi-ventricular dysfunction and heart failure. Interestingly, recent reports suggest that individuals in the acute phase can also progress to a chronic form of acute cardiomyopathy (ACM).⁴ The progression to various forms of arrhythmogenic cardiac ailments in patients with CD is multifactorial.⁵ Our prior data demonstrated a robust correlation between the loss of adipocytes and an elevated risk of cardiomyopathy in murine CD models.¹⁷ Specifically, we illustrated that the reduction in body fat resulting from adipocyte apoptosis significantly intensifies the pathogenesis of cardiomyopathy in acute and early chronic CD mice.¹¹ These findings suggest that pathological adipocytes play a pivotal role in triggering cardiomyopathy during the acute and indeterminate stages of CD. However, the molecular mechanisms underlying adipocyte loss and its impact on the myocardium and its cells, such as cardiomyocytes, fibroblasts, and resident/infiltrated immune cells during infection, have remained unclear. In particular, whether adipocyte-derived adipomes can impact myocardial functioning and regulate cardiomyopathy has never been investigated. In this study, we aimed to address these questions using various in vitro, ex vivo, and in vivo models. Our findings reveal that: (i) T. cruzi infection induces adipocyte apoptosis and increases the release of adipomes that interact with various cell types, including cardiomyocytes and immune cells; (ii) Adipomes derived from AT of T. cruzi-infected mice carry distinct lipid cargoes compared to adipomes derived from uninfected mice and regulate the expression of immune and metabolic genes in cardiomyocytes, macrophages, and fibroblasts, which are the major cell populations found in the myocardium during infection; and (iii) Plasma-derived infection-associated large adipomes (P-ILA) increase the risk of the pathogenesis of cardiomyopathy by inducing ER stress and impaired mitochondrial oxidative phosphorylation, leading to inflammation-associated cardiomyopathy in uninfected mice and apoptosis-associated cardiomyopathy in post-acute CD mice. Specifically, our study showed that P-ILA treatment triggers mitochondrial dysfunction and fosters arrhythmogenic β-AR signaling in primary cardiomyocytes. Furthermore, intracardiac P-ILA treatment was shown to induce the chronic manifestation of arrhythmic cardiomyopathy in post-acute Chagas disease (CD) mice. Another significant advancement in our study is the development of a method for isolating adipomes directly from mouse plasma and AT, which enabled the subsequent characterization of their lipid contents and functional analysis.

CD has two stages: acute and chronic, the latter of which includes asymptomatic and symptomatic stages. Although the predominant clinical manifestations of CCM typically include dilated cardiomyopathy, congestive HF, arrhythmias, and cardio embolism, some patients also exhibit features reminiscent of hypertrophic cardiomyopathy.⁴ In murine models of CD, the alteration of cardiac morphology presents differently in the acute and chronic stages of infection, mimicking the spectrum of CCM pathogenesis seen in patients. Mice with acute infection demonstrate enlarged right ventricular internal diameter (RVID) associated with LV wall thickening, resembling hypertrophic inflammatory cardiomyopathy.⁶⁸ Conversely, mice with late chronic infection exhibit increased RVID and LVID with wall thinning and decreased ejection fraction (EF %), resembling dilated cardiomyopathy with HF pre-symptoms.¹¹ We demonstrated that a loss of adipocytes influences the pathogenesis of cardiac CD using both acute and asymptomatic mice.¹¹ While in this study we utilized adipomes associated with acute T. cruzi infection to illustrate the impact of fat cell loss on hypertrophied cardiomyopathy development, these results represent a significant advancement in our general understanding of the intricate interplay among T. cruzi infection, adipocyte-derived adipomes, and myocardial function. It also offers valuable insights into the mechanisms likely disrupting immune and metabolic processes, thereby contributing to the pathogenesis of chronic cardiomyopathy in CD.

T. cruzi and other pathogens exploit AT to persist in a nutrient-rich environment and evade the immune system.^{11,6,69,70,71,72,73,74,75,76,77,78} T. cruzi lacks cholesterol biosynthesis enzymes and depends on host cholesterol, making cholesterol-rich adipocytes a preferred host cell type for invasion.¹⁹ In a previous in vivo study, we showed that T. cruzi infection induces oxidative stress and cell death in AT of infected mice.¹¹ However, previous studies did not determine whether apoptosis and/or necrosis of adipocytes were directly initiated by T. cruzi infection or indirectly triggered by immune cell-induced lipolysis in AT. In the current study, by utilizing human and murine cell lines of cultured adipocytes (in the absence of immune cells), we observed that the T. cruzi infection of cultured adipocytes induces apoptosis at 72 h post-infection. It is likely that after 96 h post-infection, T. cruzi-infected cultured adipocytes undergo necrotic cell death due to the egression of trypomastigotes after intracellular replication.⁷⁹ Adipocyte apoptosis may be a crucial phenomenon in Chagas infection during the asymptomatic stage. We have previously demonstrated that parasites persist in AT during the indeterminate and chronic stages of infection.¹⁷ During the asymptomatic stage, parasites do not vigorously replicate and establish a symbiotic relationship with host cells. However, the presence of the parasite in AT affects adipocyte physiology, leading to elevated lipolysis,²⁶ whereas impaired lipolysis is a potent trigger of adipocyte apoptosis.^{80,81} Apoptosis is known to generate apoptotic bodies (membrane-bound EVs carrying valuable biological information) as well as other EVs, including microvesicles and exosomes.^{31,32,33,82} Unlike EVs derived from other cell types, adipocyte-derived adipomes, including ApoBDs, contain large amounts of lipid metabolites alongside proteins and genetic material,^{83,84} distinguishing them as a unique subset of EVs.

In the current study, we found compelling evidence that AT of acutely infected mice releases significantly higher levels (approximately 10-fold) of large EVs, including apoptotic bodies, compared to uninfected mice. Building on this discovery, we further isolated and characterized both large and small adipomes to analyze their functions in detail ex vivo. Most previous studies of adipocyte-derived EVs used EVs from cultured adipocytes or ex vivo cultured adipocytes.^{21,22} In contrast, here we describe the first successful isolation of intact adipomes directly from mouse AT in its localized microenvironment using adiponectin as a surface marker, thus excluding EVs generated by non-adipocyte cells present in AT. We also identified distinct lipid cargoes specific to the origin and pathological condition of adipocytes. Infection-associated large adipomes (ILA) enriched with ceramides, sphingomyelins, and hexosylceramides have the potential to enhance the intracellular accumulation of sphingolipids and their derivatives in target cells, such as cardiomyocytes, fibroblasts and macrophages. This process could lead to heightened cardiotoxicity and the development of lipotoxic cardiomyopathy.^{85,86,87,88} Moreover, excessive ceramides and triglycerides in macrophages can lead to macrophage apoptosis.⁸⁹ Intracellular lipid accumulation also affects mitochondrial functions in fibroblasts and skeletal muscle.^{90,91,92} Our ex vivo data reveal that adipocyte-derived adipomes can regulate lipogenic/adipogenic, mitochondrial oxidative phosphorylation, and inflammatory signaling in target cells. Although our analysis showed subtle changes in mRNA expression between the target cells treated with different types of adipomes (CLA, ILA, CSA, and ISA), it is crucial to emphasize that the data underscore the regulatory impact of all adipomes on gene expression in target cells. Additionally, it is important to note that the ratio between adipomes and target cells, as well as the exposure time, may vary under physiological and pathological conditions in vivo. Moreover, while adipomes from uninfected mice exhibited similar effects, those from infected mice had a higher proportion of L-adipomes, resulting from adipocyte apoptosis during infection. This observation is highly relevant to the pathogenesis of cardiomyopathy in CD.

Our in vitro, ex vivo, and in vivo data reveal that adipomes regulate mitochondrial gene expression in macrophages, influencing the polarization of these cells. Ex vivo data demonstrates that adipome-treated macrophages exhibit increased mitochondrial gene expression, suggesting a shift toward oxidative phosphorylation, potentially promoting M2 polarization and anti-inflammatory signaling,⁹³ which may aid parasite persistence in the host. In vivo data showed no significant increase in the levels of pro-inflammatory cytokines (either TNFα or IFNγ) in the hearts of adipome-treated mice, although there was a significant increase in the infiltration of macrophages compared to vehicle-treated mice. These findings highlight the complex role of AT and adipomes in CD-related cardiac inflammation and remodeling. Contrary to our findings in fibroblast cells and macrophages, AT-derived adipomes from T. cruzi-infected mice did not affect adiponectin gene expression in cardiomyocytes. Instead, they increased the expression of lipogenic genes such as Pparg, Srebp1a, and Srebp1c. High levels of Srebp1c in cardiomyocytes could lead to increased mitochondrial oxidative phosphorylation, potentially causing lipotoxicity, oxidative stress and cell death.⁹⁴ Elevated oxidative phosphorylation produces ROS, inducing Tnfα expression to mitigate cellular toxicity.⁴⁶ Adipomes also induced the expression of inflammatory genes such as Tnfa and Ifng in cardiomyocytes. The endogenous production of Tnfa in cardiomyocytes promotes a predominantly pro-inflammatory response and results in apoptosis.^{95,96}

Our ex vivo and in vivo data reveal a different mechanism of lipotoxicity induced by adipome treatment in primary cardiomyocytes and the hearts of uninfected and infected mice. In primary cardiomyocytes, adipome treatment increased the expression of genes involved in adipogenic/lipogenic, mitochondrial oxidative, and inflammatory signaling that could lead to lipotoxicity and ROS-induced cell death. We observed some similarities in mRNA expression in primary cardiomyocytes to protein expression in P-ILA-treated hearts in uninfected mice, where the protein levels of lipogenic/adipogenic (perilipin, SREBP-1, adiponectin), mitochondrial oxidative phosphorylation (HSP60), and inflammatory (TNFα) signaling markers were significantly increased. In contrast, in the hearts of post-infected mice, P-ILA treatment increased lipid hydrolysis but significantly reduced the protein levels of mitochondrial oxidative phosphorylation function markers, potentially promoting apoptosis-induced cell death, as indicated by increased levels of cleaved caspase in the heart. It is essential to acknowledge, however, that the regulatory effects observed in cell lines or primary cells in vitro may differ from those observed in in vivo studies of whole animals. Such discrepancies could be due to various factors, including hormones, the presence of different immune cell populations, and metabolic regulators, all of which contribute to myocardial function.

Acute T. cruzi infection induces myocarditis with hypertrophy and fibrosis in both mice and humans. Cardiac remodeling, a crucial process during the asymptomatic phase of CD, involves the replacement of dead cardiomyocytes with fibroblasts and the resolution of pro-inflammatory immune responses, contributing to the remodeling phenomenon. However, instances of chronic cardiomyopathy have been observed in patients with acute CD,⁴ and the cause for this early response is not fully understood. Our in vivo data suggests that adipomes can impact the myocardium through either paracrine or endocrine effects during any stage of infection, depending on the pathological status of AT. This interaction could induce the infiltration of immune cells, inflammation, adipogenesis, mitochondrial dysfunction, and ER stress in the myocardium, leading to cardiomyopathy.^{9,10,11}, Cardiac fat plays a significant role in regulating metabolic functions and myocardial inflammation.^{97,98} We have observed that pericardial fat disappears in acute T. cruzi-infected mice (FIG. 11A), emphasizing its role in myocarditis and ventricular enlargement in CD. Adipocyte-specific hormone adiponectin upregulates AMPK signaling in the myocardium, which is essential for cardiovascular functions,^{99, 100} but its overexpression in Chagas mice leads to adverse effects, such as increased adipogenesis, lipotoxicity, and mitochondrial dysfunction.⁸ During the loss of healthy adipocytes, cardiac fat may not express sufficient hormones to regulate heart muscle cells, while adipomes from pathological adipocytes may influence gene expression in the heart. Our results demonstrate that adipome treatment increases adiponectin expression in fibroblasts and macrophages, which is further supported by the in vivo data showing that treatment with P-ILA elevates cardiac adiponectin levels (FIG. 9A).

Our in vivo data demonstrate the functional role of P-ILA in elevating the risk and severity of the chronic form of cardiomyopathy in post-acute CD mice, associated with mechanisms involving lipid hydrolysis, impaired mitochondrial oxidative phosphorylation, ER stress, and apoptosis. In P-ILA-treated uninfected mice, the risk of cardiomyopathy is associated with mechanisms such as lipogenesis/adipogenesis and inflammation, similar to what we observed in adipome-treated primary cardiomyocytes. However, elevated β-AR signaling, and ER stress were observed in the hearts of both uninfected and infected mice treated with P-ILA. Previously, we demonstrated that the inhibition of ER stress improves cardiac morphology and functioning in chronic CD mice.⁹ It has been shown that ER stress leads to apoptosis, inflammation, and cardiac hypertrophy.^{101,102} Additionally, chronic β-AR stimulation induces ER stress.⁴⁷ It has also been shown that ER stress causes an increase in IL6.^{103,104} Our data suggest that the increase in IL6 in the hearts is induced via ER stress in infected mice, which is exacerbated by P-ILA treatment. The chronic elevation of β1-AR in the heart is associated with cardiac hypertrophy and ventricular remodeling.^{105,106,107} Our comprehensive analysis, spanning in vitro, ex vivo, and in vivo studies, underscores the regulatory impact of adipomes on β-AR signaling, and heightened β-AR signaling has been implicated as a potential precursor to arrhythmias⁶⁷ Chagas heart disease, recognized as an arrhythmogenic cardiomyopathy, is characterized by atrial and ventricular arrhythmias, as well as a spectrum of conduction system abnormalities.⁵ While arrhythmias commonly manifest in chronic patients with dilated cardiomyopathy, instances of hypertrophied cardiomyopathy have also been documented in Chagas disease.^{108,109} Mitochondrial dysfunction promotes cardiac arrhythmias,^{48,49,50} and our data demonstrate that P-ILA treatment in cardiomyocytes causes mitochondrial dysfunction and increases β-AR signaling. Our research further establishes elevated levels of ANP in the hearts of P-ILA-treated mice, adding another marker indicative of Chagas cardiomyopathy in both uninfected and infected mice.

Our current study delves into the role of AT- and plasma-derived adipomes in the pathogenesis of cardiomyopathy. The myocardium can be regulated by exosomes derived from various cell types,^{110,111} including exosomes produced by T. cruzi.¹¹² Studies have demonstrated that T. cruzi-derived EVs exert immunomodulatory effects with potential applications in CD treatment.^{112,113} A noteworthy aspect of our study is the development of methods to isolate intact tissue-derived and circulating adipomes from mice. While several studies have used EVs derived from cultured or ex vivo adipocytes,^{22,114,115} our research provides a valuable resource for isolating intact adipomes directly from organs and tissues to explore their functional role in various diseases

### REFERENCES

1. Rassi, A., Jr., Rassi, A., and Marin-Neto, J. A. (2010). Chagas disease. Lancet 375, 1388-1402. https://doi.org/10.1016/Sβ140-6736(10)60061-X.
2. Lidani, K. C. F., Andrade, F. A., Bavia, L., Damasceno, F. S., Beltrame, M. H., Messias-Reason, I. J., and Sandri, T. L. (2019). Chagas Disease: From Discovery to a Worldwide Health Problem. Front. Public Health 7, 166. https://doi.org/10.3389/fpubh.2019.00166.
3. Cunha-Neto, E., and Chevillard, C. (2014). Chagas disease cardiomyopathy: immunopathology and genetics. Mediat. Inflamm. 2014, 683230. https://doi.org/10. 1155/2014/683230.
4. Chadalawada, S., Sillau, S., Archuleta, S., Mundo, W., Bandali, M., Parra-Henao, G., Rodriguez-Morales, A. J., Villamil-Gomez, W. E., Sua'rez, J. A., Shapiro, L., et al. (2020). Risk of Chronic Cardiomyopathy Among Patients with the Acute Phase or Indeterminate Form of Chagas Disease: A Systematic Review and Meta-analysis. JAMA Netw. Open 3, e2015072. https://doi.org/10.1001/jamanetworkopen.2020. 15072.
5. Nunes, M. C. P., Beaton, A., Acquatella, H., Bern, C., Bolger, A. F., Echevern'a, L. E., Dutra, W. O., Gascon, J., Morillo, C. A., Oliveira-Filho, J., et al. (2018). Chagas Cardiomyopathy: An Update of Current Clinical Knowledge and Management: A Scientific Statement from the American Heart Association. Circulation 138, e169-e209. https://doi.org/10.1161/CIR. 0000000000000599.
6. Camargo, E. P., Gazzinelli, R. T., Morel, C. M., and Precioso, A. R. (2022). Why do we still have not a vaccine against Chagas disease? Mem. Inst. Oswaldo Cruz 117, e200314. https://doi.org/10.1590/0074-02760200314.
7. Rocha, I. H., Ferreira Marques, A. L., Moraes, G. V., Alves da Silva, D. A., Silva, M. V. D., Rodrigues, V., Cunha, D. F. D., and Correia, D. (2020). Metabolic and immunological evaluation of patients with indeterminate and cardiac forms of Chagas disease. Medicine (Baltim.) 99, e23773. https://doi.org/10.1097/MD.0000000000023773.
8. Lizardo, K., Ayyappan, J. P., Cui, M. H., Balasubramanya, R., Jelicks, L. A., and Nagajyothi, J. F. (2019). High fat diet aggravates cardiomyopathy in murine chronic Chagas disease. Microb. Infect. 21, 63-71. https://doi.org/10.1016/j. micinf2018.07.001.
9. Ayyappan, J. P., Lizardo, K., Wang, S., Yurkow, E., and Nagajyothi, J. F. (2019). Inhibition of ER Stress by 2-Aminopurine Treatment Modulates Cardiomyopathy in a Murine Chronic Chagas Disease Model. Biomol. Ther. 27, 386-394. https://doi.org/10.4062/biomolther.2018.193.
10. Ayyappan, J. P., Lizardo, K., Wang, S., Yurkow, E., and Nagajyothi, J. F. (2020). Inhibition of SREBP Improves Cardiac Lipidopathy, Improves Endoplasmic Reticulum Stress, and Modulates Chronic Chagas Cardiomyopathy. J. Am. Heart Assoc. 9, eβ14255. https://doi.org/10.1161/JAHA.119.014255.
11. Lizardo, K., Ayyappan, J. P., Oswal, N., Weiss, L. M., Scherer, P. E., and Nagajyothi, J. F. (2021). Fat tissue regulates the pathogenesis and severity of cardiomyopathy in murine chagas disease. PLoS Neglected Trop. Dis. 15, e0008964. https://doi.org/10.1371/journal. pntd. 0008964.
12. Marelli-Berg, F. M., and Aksentijevic, D. (2019). Immunometabolic cross-talk in the inflamed heart. Cell Stress 3, 240-266. https://doi.org/10.15698/cst2019.08.194.
13. Choe, S. S., Huh, J. Y, Hwang, I. J., Kim, J. I., and Kim, J. B. (2016). Adipose Tissue Remodeling: Its Role in Energy Metabolism and Metabolic Disorders. Front. Endocrinol. 7, 30. https://doi.org/10.3389/fendo.2016. 00030.
14. Luo, L., and Liu, M. (2016). Adipose tissue in control of metabolism. J. Endocrinol. 231, R77-R99. https://doi.org/10.1530/JOE-16-0211.
15. Dragoo, J. L., Shapiro, S. A., Bradsell, H., and Frank, R. M. (2021). The essential roles of human adipose tissue: Metabolic, thermoregulatory, cellular, and paracrine effects. J. Cartil. Jt. Preserv. 1, 100023.
16. Samanta, R., Pouliopoulos, J., Thiagalingam, A., and Kovoor, P. (2016). Role of adipose tissue in the pathogenesis of cardiac arrhythmias. Heart Rhythm 13, 311-320. https://doi.org/10.1016/j.hrthm. 2015.08.016.
17. Combs, T. P., Nagajyothi, Mukherjee, S., de Almeida, C. J. G., Jelicks, L. A., Schubert, W., Lin, Y, Jayabalan, D. S., Zhao, D., Braunstein, V. L., et al. (2005). The adipocyte as an important target cell for Trypanosoma cruzi infection. J. Biol. Chem. 280, 24085-24094. https://doi.org/10.1074/jbc.M412802200.
18. Nagajyothi, F., Machado, F. S., Burleigh, B. A., Jelicks, L. A., Scherer, P. E., Mukherjee, S., Lisanti, M. P., Weiss, L. M., Garg, N.J., and Tanowitz, H. B. (2012). Mechanisms of Trypanosoma cruzi persistence in Chagas disease. Cell Microbiol. 14, 634-643. https://doi.org/10.1111/j.1462-5822.2012.01764.x.
19. Biagiotti, M., Dominguez, S., Yamout, N., and Zufferey, R. (2017). Lipidomics and anti-trypanosomatid chemotherapy. Clin. Transl. Med. 6, 27. https://doi.org/10.1186/s40169-β17-β160-7.
20. Zuzarte-Luis, V, and Mota, M. M. (2018). Parasite Sensing of Host Nutrients and Environmental Cues. Cell Host Microbe 23, 749-758. https://doi.org/10.1016/j.chom. 2018.05.018.
21. Blandin, A., Amosse, J., Froger, J., Hilairet, G., Durcin, M., Fizanne, L., Ghesquie' re, V., Prieur, X., Chaigneau, J., Vergori, L., et al. (2023). Extracellular vesicles are carriers of adiponectin with insulin-sensitizing and anti-inflammatory properties. Cell Rep. 42, 112866. https://doi.org/10.1016/j.celrep. 2023.112866.
22. Blandin, A., Dugail, I., Hilairet, G., Ponnaiah, M., Ghesquiere, V., Froger, J., Ducheix, S., Fizanne, L., Boursier, J., Cariou, B., et al. (2023). Lipidomic analysis of adipose-derived extracellular vesicles reveals specific EV lipid sorting informative of the obesity metabolic state. Cell Rep. 42, 112169. https://doi.org/10.1016/j.celrep.2023. 112169.
23. Prins, J. B., Niesler, C. U., Winterford, C. M., Bright, N. A., Siddle, K., O'Rahilly, S., Walker, N. I., and Cameron, D. P. (1997). Tumor necrosis factor-alpha induces apoptosis of human adipose cells. Diabetes 46, 1939-1944. https://doi.org/10.2337/diab.46. 12.1939.
24. Wen, J. J., Nagajyothi, F., Machado, F. S., Weiss, L. M., Scherer, P. E., Tanowitz, H. B., and Garg, N.J. (2014). Markers of oxidative stress in adipose tissue during Trypanosoma cruzi infection. Parasitol. Res. 113, 3159-3165. https://doi.org/10.1007/s00436-014-3977-7.
25. Nagajyothi, J. F., and Weiss, L. M. (2019). Advances in understanding the role of adipose tissue and mitochondrial oxidative stress in Trypanosoma cruzi infection. F1000Res. 8. F1000 Faculty Rev-1152. https://doi.org/10.12688/f1000research. 19190.1.
26. Nagajyothi, F., Desruisseaux, M. S., Machado, F. S., Upadhya, R., Zhao, D., Schwartz, G. J., Teixeira, M. M., Albanese, C., Lisanti, M. P., Chua, S. C., Jr., et al. (2012). Response of adipose tissue to early infection with Trypanosoma cruzi (Brazil strain). J. Infect. Dis. 205, 830-840. https://doi.org/10.1093/infdis/jir840.
27. Fitzgibbons, T. P., and Czech, M. P. (2014). Epicardial and perivascular adipose tissues and their influence on cardiovascular disease: basic mechanisms and clinical associations. J. Am. Heart Assoc. 3, e000582. https://doi.org/10.1161/JAHA. 113.000582.
28. Bale, L. K., West, S. A., and Conover, C. A. (2018). Characterization of mouse pericardial fat: regulation by PAPP-A. Growth Hormone IGF Res. 42-43, 1-7. https://doi.org/10.1016/j.ghir.2018.07.002.
29. Raposo, G., and Stoorvogel, W. (2013). Extracellular vesicles: exosomes, microvesicles, and friends. J. Cell Biol. 200, 373-383. https://doi.org/10.1083/jcb. 201211138.
30. Borges, F. T., Reis, L. A., and Schor, N. (2013). Extracellular vesicles: structure, function, and potential clinical uses in renal diseases. Braz. J. Med. Biol. Res. 46, 824-830. https://doi.org/10.1590/1414-431×20132964.
31. Mashouri, L., Yousefi, H., Aref, A. R., Ahadi, A. M., Molaei, F., and Alahari, S. K. (2019). Exosomes: composition, biogenesis, and mechanisms in cancer metastasis and drug resistance. Mol. Cancer 18, 75. https://doi.org/10.1186/s12943-019-0991-5.
32. Yanez-Mo, M., Siljander, P. R. M., Andreu, Z., Zavec, A. B., Borra s, F. E., Buzas, E. I., Buzas, K., Casal, E., Cappello, F., Carvalho, J., et al. (2015). Biological properties of extracellular vesicles and their physiological functions. J. Extracell. Vesicles 4, 27066. https://doi.org/10.3402/jev.v4.27066.
33. Kakarla, R., Hur, J., Kim, Y. J., Kim, J., and Chwae, Y. J. (2020). Apoptotic cell-derived exosomes: messages from dying cells. Exp. Mol. Med. 52, 1-6. https://doi.org/10.1038/s12276-β19-0362-8.
34. Stewart, S., Gessler, F., Pluchino, S., and Moreau, K. (2016). Inside-out: Unpredicted Annexin A2 Localisation on the Surface of Extracellular Vesicles (ScienceMatters). https://doi.org/10.19185/matters. 201602000015.
35. Stewart, S. E., Ashkenazi, A., Williamson, A., Rubinsztein, D. C., and Moreau, K. (2018). Phospholipid flipping facilitates annexin translocation across membranes. Preprint at bioRxiv. https://doi.org/10.1101/241976.
36. Shah, N., Ishii, M., Brandon, C., Ablonczy, Z., Cai, J., Liu, Y, Chou, C. J., and Rohrer, B. (2018). Extracellular vesicle-mediated long-range communication in stressed retinal pigment epithelial cell monolayers. Biochim. Biophys. Acta, Mol. Basis Dis. 1864, 2610-2622. https://doi.org/10.1016/j. bbadis.2018.04.016.
37. Botha, J., Handberg, A., and Simonsen, J. B. (2022). Lipid-based strategies used to identify extracellular vesicles in flow cytometry can be confounded by lipoproteins: Evaluations of annexin V, lactadherin, and detergent lysis. J. Extracell. Vesicles 11, e12200. https://doi.org/10. 1002/jev2.12200.
38. Lee, J. E., Moon, P. G., Lee, I. K., and Baek, M. C. (2015). Proteomic Analysis of Extracellular Vesicles Released by Adipocytes of Otsuka Long-Evans Tokushima Fatty (OLETF) Rats. Protein J. 34, 220-235. https://doi.org/10.1007/s10930-β15-9616-z.
39. Stroh, A. M., Lynch, C. E., Lester, B. E., Minchev, K., Chambers, T. L., Montenegro, C. F., Chavez Martinez, C., Fountain, W. A., Trappe, T. A., and Trappe, S. W. (2021). Human adipose and skeletal muscle tissue DNA, RNA, and protein content. J. Appl. Physiol. 131, 1370-1379. https://doi.org/10. 1152/japplphysiol.00343.2021.
40. Chen, H., Wang, Z., Qin, M., Zhang, B., Lin, L., Ma, Q., Liu, C., Chen, X., Li, H., Lai, W., and Zhong, S. (2021). Comprehensive Metabolomics Identified the Prominent Role of Glycerophospholipid Metabolism in Coronary Artery Disease Progression. Front. Mol. Biosci. 8, 632950. https://doi.org/10. 3389/fmolb.2021.632950.
41. Borodzicz-Jaz_dz_yk, S., Jaz_dz_yk, P., Lysik, W., Cudnoch-Je, drzejewska, A., and Czarzasta, K. (2022). Sphingolipid metabolism and signaling in cardiovascular diseases. Front. Cardiovasc. Med. 9, 915961. https://doi.org/10.3389/fcvm.2022.915961.
42. Sun, H., Sun, S., Chen, G., Xie, H., Yu, S., Lin, X., Qian, J., Mao, C., Peng, H., Chen, H., et al. (2021). Ceramides and sphingosine-1-phosphate mediate the distinct effects of M1/M2-macrophage infusion on liver recovery after hepatectomy. Cell Death Dis. 12, 324. https://doi.org/10.1038/s41419-021-03616-9.
43. Aflaki, E., Radovic, B., Chandak, P. G., Kolb, D., Eisenberg, T., Ring, J., Fertschai, I., Uellen, A., Wolinski, H., Kohlwein, S. D., et al. (2011). Triacylglycerol accumulation activates the mitochondrial apoptosis pathway in macrophages. J. Biol. Chem. 286, 7418-7428. https://doi.org/10.1074/jbc.M110.175703.
44. Von Bank, H., Hurtado-Thiele, M., Oshimura, N., and Simcox, J. (2021). Mitochondrial Lipid Signaling and Adaptive Thermogenesis. Metabolites 11, 124. https://doi.org/10.3390/metabol1020124.
45. Lizaso, A., Tan, K. T., and Lee, YH. (2013). beta-adrenergic receptor-stimulated lipolysis requires the RAB7-mediated autolysosomal lipid degradation. Autophagy 9, 1228-1243. https://doi.org/10.4161/auto.24893.
46. Blaser, H., Dostert, C., Mak, T. W., and Brenner, D. (2016). TNF and ROS Crosstalk in Inflammation. Trends Cell Biol. 26, 249-261. https://doi.org/10.1016/j.tcb. 2015.12.002.
47. Dalal, S., Foster, C. R., Das, B. C., Singh, M., and Singh, K. (2012). Beta-adrenergic receptor stimulation induces endoplasmic reticulum stress in adult cardiac myocytes: role in apoptosis. Mol. Cell. Biochem. 364, 59-70. https://doi.org/10.1007/s11010-011-1205-7.
48. Deng, J., Jiang, Y, Chen, Z. B., Rhee, J. W., Deng, Y, and Wang, Z. V. (2023). Mitochondrial Dysfunction in Cardiac Arrhythmias. Cells 12, 679. https://doi.org/10.3390/cells12050679.
49. Yang, K. C., Bonini, M. G., and Dudley, S. C., Jr. (2014). Mitochondria and arrhythmias. Free Radic. Biol. Med. 71, 351-361. https://doi.org/10.1016/j.freeradbiomed.2014. 03.033.
50. Saadeh, K., and Fazmin, I. T. (2021). Mitochondrial Dysfunction Increases Arrhythmic Triggers and Substrates, Potential Anti-arrhythmic Pharmacological Targets. Front. Cardiovasc. Med. 8, 646932. https://doi.org/10.3389/fcvm.2021.646932.
51. Vinhas, M., Arau'jo, A. C., Ribeiro, S., Rosa'rio, L. B., and Belo, J. A. (2013). Transthoracic echocardiography reference values in juvenile and adult 129/Sv mice. Cardiovasc. Ultrasound 11, 12. https://doi.org/10.1186/1476-7120-11-12.
52. Sorensen, L. L., Bedja, D., Sysa-Shah, P., Liu, H., Maxwell, A., Yi, X., Pozios, I., Olsen, N. T., Abraham, T. P., Abraham, R., and Gabrielson, K. (2016). Echocardiographic Characterization of a Murine Model of Hypertrophic Obstructive Cardiomyopathy Induced by Cardiac-specific Overexpression of Epidermal Growth Factor Receptor 2. Comp. Med. 66, 268-277.
53. AHA (2023). Ejection Fraction Heart Failure Measurement. https://www.heart.org/en/health-topics/heart-failure/diagnosing-heart-failure/ejection-fraction-heart-failure-measurement.
54. Xu, Y, Zhang, Y, and Ye, J. (2018). IL-6: A Potential Role in Cardiac Metabolic Homeostasis. Int. J. Mol. Sci. 19, 2474. https://doi.org/10.3390/ijms19092474.
55. van Hall, G., Steensberg, A., Sacchetti, M., Fischer, C., Keller, C., Schjerling, P., Hiscock, N., Møller, K., Saltin, B., Febbraio, M. A., and Pedersen, B. K. (2003). Interleukin-6 stimulates lipolysis and fat oxidation in humans. J. Clin. Endocrinol. Metab. 88, 3005-3010. https://doi.org/10.1210/jc.2002-021687.
56. Marcinkiewicz, A., Gauthier, D., Garcia, A., and Brasaemle, D. L. (2006). The phosphorylation of serine 492 of perilipin a directs lipid droplet fragmentation and dispersion. J. Biol. Chem. 281, 11901-11909. https://doi.org/10.1074/jbc.M600171200.
57. Bougarne, N., Weyers, B., Desmet, S. J., Deckers, J., Ray, D. W., Staels, B., and De Bosscher, K. (2018). Molecular Actions of PPARalpha in Lipid Metabolism and Inflammation. Endocr. Rev. 39, 760-802. https://doi.org/10.1210/er.2018-00064.
58. Guerra, I. M. S., Ferreira, H. B., Melo, T., Rocha, H., Moreira, S., Diogo, L., Domingues, M. R., and Moreira, A. S. P. (2022). Mitochondrial Fatty Acid beta-Oxidation Disorders: From Disease to Lipidomic Studies-A Critical Review. Int. J. Mol. Sci. 23, 13933. https://doi.org/10.3390/ijms232213933.
59. Cnop, M., Toivonen, S., Igoillo-Esteve, M., and Salpea, P. (2017). Endoplasmic reticulum stress and eIF2alpha phosphorylation: The Achilles heel of pancreatic beta cells. Mol. Metabol. 6, 1024-1039. https://doi.org/10.1016/j. molmet.2017.06.001.
60. Szegezdi, E., Logue, S. E., Gorman, A. M., and Samali, A. (2006). Mediators of endoplasmic reticulum stress-induced apoptosis. EMBO Rep. 7, 880-885. https://doi.org/10.1038/sj.embor.7400779.
61. Oslowski, C. M., and Urano, F. (2011). Measuring ER stress and the unfolded protein response using mammalian tissue culture system. Methods Enzymol. 490, 71-92. https://doi.org/10.1016/B978-O-12-385114-7.00004-0.
62. Chandra, D., Choy, G., and Tang, D. G. (2007). Cytosolic accumulation of HSP60 during apoptosis with or without apparent mitochondrial release: evidence that its pro-apoptotic or pro-survival functions involve differential interactions with caspase-3. J. Biol. Chem. 282, 31289-31301. https://doi.org/10.1074/jbc.M702777200.
63. Martinus, R. D., and Goldsbury, J. (2018). Endothelial TNF-alpha induction by Hsp60 secreted from THP-1 monocytes exposed to hyperglycaemic conditions. Cell Stress Chaperones 23, 519-525. https://doi.org/10.1007/s12192-017-0858-x.
64. Labovsky, V., Smulski, C. R., Go'mez, K., Levy, G., and Levin, M. J. (2007). Anti-beta1-adrenergic receptor autoantibodies in patients with chronic Chagas heart disease. Clin. Exp. Immunol. 148, 440-449. https://doi.org/10.1111/j.1365-2249.2007.03381.x.
65. Higuchi, M. d. L., Benvenuti, L. A., Martins Reis, M., and Metzger, M. (2003). Pathophysiology of the heart in Chagas' disease: current status and new developments. Cardiovasc. Res. 60, 96-107. https://doi.org/10.1016/s0008-6363(03) 00361-4.
66. Yoo, B., Lemaire, A., Mangmool, S., Wolf, M. J., Curcio, A., Mao, L., and Rockman, H. A. (2009). Betal-adrenergic receptors stimulate cardiac contractility and CaMKII activation in vivo and enhance cardiac dysfunction following myocardial infarction. Am. J. Physiol. Heart Circ. Physiol. 297, H1377-H1386. https://doi.org/10.1152/ajpheart.00504.2009.
67. Zhang, Y, Hou, M. C., Li, J. J., Qi, Y, Zhang, Y, She, G., Ren, Y. J., Wu, W., Pang, Z. D., Xie, W., et al. (2020). Cardiac beta-adrenergic receptor activation mediates distinct and cell type-dependent changes in the expression and distribution of connexin 43. J. Cell Mol. Med. 24, 8505-8517. https://doi.org/10.1111/jcmm.15469.
68. Nagajyothi, F., Weiss, L. M., Zhao, D., Koba, W., Jelicks, L. A., Cui, M. H., Factor, S. M., Scherer, P. E., and Tanowitz, H. B. (2014). High fat diet modulates Trypanosoma cruzi infection associated myocarditis. PLoS Neglected Trop. Dis. 8, e3118. https://doi. org/10.1371/journal. pntd.0003118.
69. Trindade, S., Rijo-Ferreira, F., Carvalho, T., Pinto-Neves, D., Guegan, F., Aresta-Branco, F., Bento, F., Young, S. A., Pinto, A., Van Den Abbeele, J., et al. (2016). Trypanosoma brucei Parasites Occupy and Functionally Adapt to the Adipose Tissue in Mice. Cell Host Microbe 19, 837-848. https://doi.org/10.1016/j.chom.2016.05.002.
70. Ayyappan, J. P., and Nagajyothi, J. F. (2017). Diet Modulates Adipose Tissue Oxidative Stress in a Murine Acute Chagas Model. JSM Atheroscler. 2, 1030.
71. Lizardo, K., Almonte, V., Law, C., Aiyyappan, J. P., Cui, M. H., and Nagajyothi, J. F. (2017). Diet regulates liver autophagy differentially in murine acute Trypanosoma cruzi infection. Parasitol. Res. 116, 711-723. https://doi.org/10.1007/s00436-016-5337-2.
72. Beigier-Bompadre, M., Montagna, G. N., Ku″hl, A. A., Lozza, L., Weiner, J., 3rd, Kupz, A., Vogelzang, A., Mollenkopf, H. J., Lo″we, D., Bandermann, S., et al. (2017). Mycobacterium tuberculosis infection modulates adipose tissue biology. PLoS Pathog. 13, e1006676. https://doi.org/10. 1371/journal.ppat.1006676.
73. Ayyappan, J. P., Vinnard, C., Subbian, S., and Nagajyothi, J. F. (2018). Effect of Mycobacterium tuberculosis infection on adipocyte physiology. Microb. Infect. 20, 81-88. https://doi.org/10.1016/j. micinf. 2017.10.008.
74. Gonzalez, F. B., Villar, S. R., Toneatto, J., Pacini, M. F., Marquez, J., D'Attilio, L., Bottasso, O. A., Piwien-Pilipuk, G., and Perez, A. R. (2019). Immune response triggered by Trypanosoma cruzi infection strikes adipose tissue homeostasis altering lipid storage, enzyme profile and adipokine expression. Med. Microbiol. Immunol. 208, 651-666. https://doi.org/10.1007/s00430-β18-0572-z.
75. Ayyappan, J. P., Ganapathi, U., Lizardo, K., Vinnard, C., Subbian, S., Perlin, D. S., and Nagajyothi, J. F. (2019). Adipose Tissue Regulates Pulmonary Pathology during TB Infection. mBio 10, eβ2771-18. https://doi.org/10.1128/mBio.β2771-18.
76. Dhanyalayam, D., Thangavel, H., Lizardo, K., Oswal, N., Dolgov, E., Perlin, D. S., and Nagajyothi, J. F. (2022). Sex Differences in Cardiac Pathology of SARS-CoV2 Infected and Trypanosoma cruzi Co-infected Mice. Front. Cardiovasc. Med. 9, 783974. https://doi.org/10.3389/fcvm.2022.783974.
77. Martinez-Colon, G. J., Ratnasiri, K., Chen, H., Jiang, S., Zanley, E., Rustagi, A., Verma, R., Chen, H., Andrews, J. R., Mertz, K. D., et al. (2022). SARS-CoV-2 infection drives an inflammatory response in human adipose tissue through infection of adipocytes and macrophages. Sci. Transl. Med. 14, eabm9151. https://doi.org/10.1126/scitranslmed.abm9151.
78. Thangavel, H., Dhanyalayam, D., Lizardo, K., Oswal, N., Dolgov, E., Perlin, D. S., and Nagajyothi, J. F. (2023). Susceptibility of Fat Tissue to SARS-CoV-2 Infection in Female hACE2 Mouse Model. Int. J. Mol. Sci. 24, 1314. https://doi.org/10.3390/ijms24021314.
79. Sykes, M. L., Kennedy, E. K., and Avery, V. M. (2023). Impact of Laboratory-Adapted Intracellular Trypanosoma cruzi Strains on the Activity Profiles of Compounds with Anti-T. cruzi Activity. Microorganisms 11, 476. https://doi.org/10.3390/microorganisms11020476.
80. Jung, T. W., Park, T., Park, J., Kim, U., Je, H. D., Kim, H. D., Cho, S. W., Abd E1-Aty, A. M., Song, J. H., Kim, H. C., et al. (2019). Phosphatidylcholine causes adipocyte-specific lipolysis and apoptosis in adipose and muscle tissues. PLoS One 14, eβ214760. https://doi.org/10.1371/journal.pone. β214760.
81. Roszer, T. (2021). Adipose Tissue Immunometabolism and Apoptotic Cell Clearance. Cells 10, 2288. https://doi.org/10.3390/ce11s10092288.
82. Barros, F. M., Carneiro, F., Machado, J. C., and Melo, S. A. (2018). Exosomes and Immune Response in Cancer: Friends or Foes? Front. Immunol. 9, 730. https://doi. org/10.3389/fimmu.2018.00730.
83. Durcin, M., Fleury, A., Taillebois, E., Hilairet, G., Krupova, Z., Henry, C., Truchet, S., Tro″tzmu″ller, M., Ko″feler, H., Mabilleau, G., et al. (2017). Characterisation of adipocyte-derived extracellular vesicle subtypes identifies distinct protein and lipid signatures for large and small extracellular vesicles. J. Extracell. Vesicles 6, 1305677. https://doi.org/10.1080/20013078.2017. 1305677.
84. Rome, S., Blandin, A., and Le Lay, S. (2021). Adipocyte-Derived Extracellular Vesicles: State of the Art. Int. J. Mol. Sci. 22, 1788. https://doi.org/10.3390/ijms22041788.
85. Kovilakath, A., and Cowart, L. A. (2020). Sphingolipid Mediators of Myocardial Pathology. J. Lipid Atheroscler. 9, 23-49. https://doi.org/10.12997/jla.2020.9.1.23.
86. Park, T. S., Hu, Y, Noh, H. L., Drosatos, K., Okajima, K., Buchanan, J., Tuinei, J., Homma, S., Jiang, X. C., Abel, E. D., and Goldberg, I. J. (2008). Ceramide is a cardiotoxin in lipotoxic cardiomyopathy. J. Lipid Res. 49, 2101-2112. https://doi.org/10.1194/jlr. M800147-JLR200.
87. Bekhite, M., Gonza'lez-Delgado, A., Hu″bner, S., Haxhikadrija, P., Kretzschmar, T., Muller, T., Wu, J. M. F., Bekfani, T., Franz, M., Wartenberg, M., et al. (2021). The role of ceramide accumulation in human induced pluripotent stem cell-derived cardiomyocytes on mitochondrial oxidative stress and mitophagy. Free Radic. Biol. Med. 167, 66-80. https://doi.org/10.1016/j. freeradbiomed.2021.02.016.
88. Shu, H., Peng, Y, Hang, W., Li, N., Zhou, N., and Wang, D. W. (2022). Emerging Roles of Ceramide in Cardiovascular Diseases. Aging Dis. 13, 232-245. https://doi.org/10. 14336/AD.2021.0710.
89. Aflaki, E., Doddapattar, P., Radovi, c, B., Povoden, S., Kolb, D., Vuji, c, N., Wegscheider, M., Koefeler, H., Hornemann, T., Graier, W. F., et al. (2012). C16 ceramide is crucial for triacylglycerol-induced apoptosis in macrophages. Cell Death Dis. 3, e280. https://doi.org/10.1038/cddis.2012.17.
90. Jennings, M. J., Hathazi, D., Nguyen, C. D. L., Munro, B., Mu″nchberg, U., Ahrends, R., Schenck, A., Eidhof, I., Freier, E., Synofzik, M., et al. (2021). Intracellular Lipid Accumulation and Mitochondrial Dysfunction Accompanies Endoplasmic Reticulum Stress Caused by Loss of the Co-chaperone DNAJC3. Front. Cell Dev. Biol. 9, 710247. https://doi.org/10.3389/fcell.2021. 710247.
91. Kim, J. A., Wei, Y, and Sowers, J. R. (2008). Role of mitochondrial dysfunction in insulin resistance. Circ. Res. 102, 401-414. https://doi.org/10.1161/CIRCRESAHA.107.165472.
92. Kratky, D., Obrowsky, S., Kolb, D., and Radovic, B. (2014). Pleiotropic regulation of mitochondrial function by adipose triglyceride lipase-mediated lipolysis. Biochimie 96, 106-112. https://doi.org/10. 1016/j.biochi.2013.06.023.
93. Viola, A., Munari, F., Sanchez-Rodri'guez, R., Scolaro, T., and Castegna, A. (2019). The Metabolic Signature of Macrophage Responses. Front. Immunol. 10, 1462. https://doi.org/10.3389/fimmu.2019.01462.
94. Ferre, P., Phan, F., and Foufelle, F. (2021). SREBP-1c and lipogenesis in the liver: an updatel. Biochem. J. 478, 3723-3739. https://doi.org/10.1042/BCJ20210071.
95. Chen, Y, Pat, B., Zheng, J., Cain, L., Powell, P., Shi, K., Sabri, A., Husain, A., and Dell'italia, L. J. (2010). Tumor necrosis factor-alpha produced in cardiomyocytes mediates a predominant myocardial inflammatory response to stretch in early volume overload. J. Mol. Cell. Cardiol. 49, 70-78. https://doi.org/10.1016/j.yjmcc. 2009.12.013.
96. Li, Z., Zhou, J., Zhu, D., Zhang, Q., Huang, M., Han, Y, and Zhou, S. (2015). Role of endogenous TNF-α in cardiomyocyte apoptosis induced by bacteria lipoprotein and the protective effect of IL-10. Eur. J. Inflamm. 13, 117-125. https://doi.org/10. 1177/1721727×15597363.
97. Gandoy-Fieiras, N., Gonzalez-Juanatey, J. R., and Eiras, S. (2020). Myocardium Metabolism in Physiological and Pathophysiological States: Implications of Epicardial Adipose Tissue and Potential Therapeutic Targets. Int. J. Mol. Sci. 21, 2641. https://doi.org/10.3390/ijms21072641.
98. Wu, Y, Zhang, A., Hamilton, D. J., and Deng, T. (2017). Epicardial Fat in the Maintenance of Cardiovascular Health. Methodist Debakey Cardiovasc. J. 13, 20-24. https://doi.org/10.14797/mdcj-13-1-20.
99. Hopkins, T. A., Ouchi, N., Shibata, R., and Walsh, K. (2007). Adiponectin actions in the cardiovascular system. Cardiovasc. Res. 74, 11-18. https://doi.org/10.1016/j.cardiores. 2006.10.009.
100. Ouchi, N., Shibata, R., and Walsh, K. (2006). Cardioprotection by adiponectin. Trends Cardiovasc. Med. 16, 141-146. https://doi.org/10.1016/j.tcm.2006.03.001.
101. Bennett, M. R. (2002). Apoptosis in the cardiovascular system. Heart 87, 480-487. https://doi.org/10.1136/heart.87.5.480.
102. Ni, L., Zhou, C., Duan, Q., Lv, J., Fu, X., Xia, Y, and Wang, D. W. (2011). beta-AR blockers suppresses ER stress in cardiac hypertrophy and heart failure. PLoS One 6, e27294. https://doi.org/10.1371/journal.pone. 0027294.
103. Hosoi, T., Honda, M., Oba, T., and Ozawa, K. (2013). ER stress upregulated PGE(2)/IFNgamma-induced IL-6 expression and down-regulated iNOS expression in glial cells. Sci. Rep. 3, 3388. https://doi.org/10. 1038/srep03388.
104. Sanchez, C. L., Sims, S. G., Nowery, J. D., and Meares, G. P. (2019). Endoplasmic reticulum stress differentially modulates the IL-6 family of cytokines in murine astrocytes and macrophages. Sci. Rep. 9, 14931. https://doi.org/10.1038/s41598-019-51481-6.
105. Nash, C. A., Wei, W., Irannejad, R., and Smrcka, A. V. (2019). Golgi localized beta1-adrenergic receptors stimulate Golgi PI4P hydrolysis by PLCepsilon to regulate cardiac hypertrophy. Elife 8, e48167. https://doi.org/10.7554/eLife.48167.
106. Zimmer, H. G., Kolbeck-Ruhmkorff, C., and Zierhut, W. (1995). Cardiac hypertrophy induced by alpha- and beta-adrenergic receptor stimulation. Cardioscience 6, 47-57.
107. Osadchii, O. E. (2007). Cardiac hypertrophy induced by sustained beta-adrenoreceptor activation: pathophysiological aspects. Heart Fail. Rev. 12, 66-86. https://doi.org/10.1007/s10741-007-9007-4.
108. Cardoso, C. S., Sabino, E. C., Oliveira, C. D. L., de Oliveira, L. C., Ferreira, A. M., Cunha-Neto, E., Bierrenbach, A. L., Ferreira, J. E., Haikal, D. S., Reingold, A. L., and Ribeiro, A. L. P. (2016). Longitudinal study of patients with chronic Chagas cardiomyopathy in Brazil (SaMi-Trop project): a cohort profile. BMJ Open 6, eβ11181. https://doi.org/10. 1136/bmjopen-2016-011181.
109. Pastore, C. A., Samesima, N., Pereira Filho, H. G., Varoni, L. P. C., Rochitte, C. E., Vieira, M. L., Avila, L. F. R.d., Melo, R.d. J. L., Pereira, A. d. C., Daheer, J., and Carlo, C. H. d. (2015). Rare Association: Chagas' Disease and Hypertrophic Cardiomyopathy. Ann. Noninvasive Electrocardiol. 20, 498-501. https://doi.org/10.1111/anec.12229.
110. Liu, Y, Wang, M., Yu, Y, Li, C., and Zhang, C. (2023). Advances in the study of exosomes derived from mesenchymal stem cells and cardiac cells for the treatment of myocardial infarction. Cell Commun. Signal. 21, 202. https://doi.org/10.1186/si2964-023-01227-9.
111. Liang, B., He, X., Zhao, Y. X., Zhang, X. X., and Gu, N. (2020). Advances in Exosomes Derived from Different Cell Sources and Cardiovascular Diseases. BioMed Res. Int. 2020, 7298687. https://doi.org/10.1155/2020/7298687.
112. Cortes-Serra, N., Gualdron-Lopez, M., Pinazo, M. J., Torrecilhas, A. C., and Fernandez-Becerra, C. (2022). Extracellular Vesicles in Trypanosoma cruzi Infection: Immunomodulatory Effects and Future Perspectives as Potential Control Tools against Chagas Disease. J. Immunol. Res. 2022, 5230603. https://doi.org/10.1155/2022/5230603.
113. D'Avila, H., de Souza, N. P., Albertoni, A. L. D. S., Campos, L. C., Rampinelli, P. G., Correa, J. R., and de Almeida, P. E. (2021). Impact of the Extracellular Vesicles Derived From Trypanosoma cruzi: A Paradox in Host Response and Lipid Metabolism Modulation. Front. Cell. Infect. Microbiol. 11, 768124. https://doi.org/10.3389/fcimb. 2021.768124.
114. Kulaj, K., Harger, A., Bauer, M., Caliskan, O. S., Gupta, T. K., Chiang, D. M., Milbank, E., Reber, J., Karlas, A., Kotzbeck, P., et al. (2023). Adipocyte-derived extracellular vesicles increase insulin secretion through transport of insulinotropic protein cargo. Nat. Commun. 14, 709. https://doi.org/10. 1038/s41467-023-36148-1.
115. Gesmundo, I., Pardini, B., Gargantini, E., Gamba, G., Birolo, G., Fanciulli, A., Banfi, D., Congiusta, N., Favaro, E., Deregibus, M. C., et al. (2021). Adipocyte-derived extracellular vesicles regulate survival and function of pancreatic beta cells. JCI Insight 6, e141962. https://doi.org/10.1172/jci.insight.141962.
116. Schneider, C. A., Rasband, W. S., and Eliceiri, K. W. (2012). NIH Image to ImageJ: 25 years of image analysis. Nat. Methods 9, 671-675. https://doi.org/i0.1038/nmeth.2089.
117. Pang, Z., Chong, J., Zhou, G., de Lima Morais, D. A., Chang, L., Barrette, M., Gauthier, C., Jacques, P. E′., Li, S., and Xia, J. (2021). MetaboAnalyst 5.0: narrowing the gap between raw spectra and functional insights. Nucleic Acids Res. 49, W388-W396. https://doi.org/10.1093/nar/gkab382.
118. Mott, A., Lenormand, G., Costales, J., Fredberg, J. J., and Burleigh, B. A. (2009). Modulation of host cell mechanics by Trypanosoma cruzi. J. Cell. Physiol. 218, 315-322. https://doi.org/10.1002/jcp.21606.
119. Zhao, D., Lizardo, K., Cui, M. H., Ambadipudi, K., Lora, J., Jelicks, L. A., and Nagajyothi, J. F. (2016). Antagonistic effect of atorvastatin on high fat diet induced survival during acute Chagas disease. Microb. Infect. 18, 675-686. https://doi.org/10.1016/j. micinf.2016.06.006.
120. Thangavel, H., Lizardo, K., Dhanyalayam, D., De Assis, S., and Nagajyothi, J. F. (2023). Diets Differently Regulate Tumorigenesis in Young E0771 Syngeneic Breast Cancer Mouse Model. J. Clin. Med. 12, 413. https://doi.org/10.3390/jcm12020413.
121. Gupta, S., and Knowlton, A. A. (2007). HSP60 trafficking in adult cardiac myocytes: role of the exosomal pathway. Am. J. Physiol. Heart Circ. Physiol. 292, H3052-H3056. https://doi. org/10.1152/ajpheart.β1355.2006.
122. Malonis, R. J., Georgiev, G. I., Haslwanter, D., VanBlargan, L. A., Fallon, G., Vergnolle, O., Cahill, S. M., Harris, R., Cowburn, D., Chandran, K., et al. (2022). A Powassan virus domain III nanoparticle immunogen elicits neutralizing and protective antibodies in mice. PLoS Pathog. 18, e1010573. https://doi.org/10.1371/joumal. ppat.1010573.
123. Mitchell, M. I., Ben-Dov, I. Z., Liu, C., Ye, K., Chow, K., Kramer, Y, Gangadharan, A., Park, S., Fitzgerald, S., Ramnauth, A., et al. (2021). Extracellular Vesicle Capture by AnTibody of CHoice and Enzymatic Release (EV-CATCHER): A customizable purification assay designed for small-RNA biomarker identification and evaluation of circulating small-EVs. J. Extracell. Vesicles 10, e12110. https://doi.org/10.1002/jev2.12110.
124. Toivonen, R., Koskenvuo, J., Merentie, M., So″derstro m, M., Yla″-Herttuala, S., and Savontaus, M. (2012). Intracardiac injection of a capsid-modified Ad5/35 results in decreased heart toxicity when compared to standard Ad5. Virol. J. 9, 296. https://doi.org/10.1186/1743-422X-9-296.

### Example 2. Progress in Adipome Isolation and Characterization from Clinical Samples and Murine Breast Cancer Models

A pilot study was conducted to isolate large and small adipomes from the circulation using de-identified plasma samples obtained from breast cancer patients (both triple-negative breast cancer (TNBC) and hormone receptor positive (HR+) subtypes) at early (Stages I-II) and late (Stages III-IV) disease stages.

Method: Plasma samples obtained from healthy and breast cancer subjects were treated with thrombin for de-fibrination and pre-cleared by centrifugation prior to adipome isolation. Plasma adipomes were isolated and enriched using a combination of ExoQuick Ultra EV isolation kit for serum/plasma and Basic Exo-Flow Capture kit as described previously. These plasma-derived large adipomes were used for TEM characterization and lipidomic analyses to derive disease state-specific (healthy vs cancer) and disease stage-specific (early vs metastatic) lipid signatures.

Lipidomic profiling of large adipomes revealed stage-specific differences in lipid composition between early- and late-stage patient groups (FIG. 18A, FIG. 18B, FIG. 18C, FIG. 18D). Adipomes isolated from the plasma of women with breast cancer and their lipid cargoes differ in women with localized, early-stage cancer vs metastatic disease FIG. 18A Transmission Electronic Microscopy (negative) image of adipomes from plasma from a breast cancer (BC) patient. Mean adipome size is 500 nm (bar-100 nm). FIG. 18B is a PCA score plot of lipids with samples colored according to their respective groups (pink dots-early breast tumor; green dots-metastases; blue dots-healthy). The principal components showed that the PC1 accounted for 21.7% of the total variance and the PC2 accounted for 11.4%. The heatmap in FIG. 18C shows lipidomic profiles of the top 10 lipids of adipomes from plasma of early tumor subjects (red bar) differs from that of healthy subjects (green bar). The heatmap in FIG. 18D shows lipidomic profiles of the top 10 lipids of adipomes from plasma of early tumor subjects (red bar) differs from those patients with metastatic BC (green bar). (n=4-5/group).

Large and small adipomes were also isolated from tumor-associated mammary fat (TAMF) of E0771 tumor-bearing C57BL/6 mice and from normal mammary fat (MF) of wild-type mice.

Method: Adipomes from murine normal mammary fat (MF) and tumor-associated mammary fat (TAMF) were isolated and enriched using a combination of ExoQuick-TC Ultra EV isolation kit and Basic Exo-Flow Capture kit as follows. The harvested tissues (MF or TAMF) were placed in a 12-well plate and gently dissociated in presence of 500 μL cold 1×PBS (sterile) using sterile forceps and scissor/scalpel to liberate EVs present in the interstitial space. Upon dissociation, the biofluid was passed through 70 m cell strainer to remove large chunks of tissue and flushed with additional 500 μL of cold PBS. The biofluid was then centrifuged at 500×g for 10 mins to remove cell debris at 10° C. The supernatant was transferred to a new tube and re-centrifuged at 21,000×g for an hour at 10° C. After completion of the spin, the supernatant was carefully transferred again to a new tube without disturbing the pellet and/or the white precipitates smeared on the inner sides of the tube, if using fixed-angle rotor. The pellet/precipitate was resuspended in 250 μL PBS. Then, the ExoQuick-TC exosome precipitation solution was added to both the resuspended pellet and supernatant fraction at 1:5, mixed thoroughly by inverting the tubes and incubated overnight at 4° C. On the following day, the samples were centrifuged at 3,000×g for 10 minutes to pellet the precipitated EVs and further purified using the SEC columns. The EVs from resuspended pellets were referred as large-EVs (L-EV) while the supernatant derived EVs were referred as small-EVs (S-EV). Next, L-adipomes and S-adipomes were enriched from L-EV and S-EV fraction, respectively using biotinylated mouse adiponectin+ FABP4 antibody cocktail and Exo-Flow streptavidin magnetic bead system following manufacturer's instructions with slight modification to improve recovery. Washed bead-bound L-adipomes were sent for lipidomics analysis. The eluted L-adipomes were re-precipitated with ExoQuick-TC precipitation buffer for overnight at 4° C. to remove the elution buffer, centrifuged at 3,000×g for 20 minutes at 4° C., resuspended in 1×PBS and used for adipome characterization (size distribution, concentration, and TEM) and functional analyses in E0771 murine breast tumor cells.

Results. We utilized a syngeneic E0771/C57BL/6 murine breast cancer model (8 weeks old, n=16), which demonstrated metastatic progression by 3 weeks post-tumor implantation (WPI). Histological analysis (FIG. 19A) revealed that the mammary fat pad (left panel), initially composed of mature adipocytes, progressively transitioned into fibrotic tissue by 5 WPI (right panel, red arrow), concurrent with a marked loss of adipocytes. At 3 WPI (middle panel), H&E staining of tumor-associated mammary fat (TAMF) showed signs of adipocyte degradation (adipolysis) at the invasive tumor front (black arrow). The inset highlights a magnified view of adipose tissue adjacent to the tumor, illustrating adipocyte breakdown. Western blot analysis (FIG. 19B) demonstrated increased expression of apoptotic markers-cleaved caspase-3, annexin V, and Bnip3 (a Bcl2-interacting protein), in TAMF at 3 WPI relative to pre-tumoral mammary fat (MF). Protein levels were normalized to β-actin (n=3; p<0.01). Transmission electron microscopy (TEM) confirmed the successful isolation of large adipomes (L-adipomes, FIG. 19C) and small adipomes (S-adipomes, FIG. 19D) from TAMF at 3 WPI. L-adipomes measured approximately 350 nm in diameter, whereas S-adipomes were ˜40 nm. Scale bar=80 nm.

Lipidomic analysis revealed distinct lipid signatures between TAMF- and MF-derived L-adipomes. FIG. 20 is a Volcano plot showing lipids of L-adipomes isolated from TAMF vs. ME. Y-axis represents log 10β-value and x-axis log 2FC. Among the enriched lipids were palmitic acid containing PA and PS (encircled) in TAMF-adipomes.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for early diagnosis of a subject at risk for a pathology comprising alterations in metabolic and inflammatory signaling in phagocytic and nonphagocytic target cells, the method comprising:
selectively purifying from a population of adipocytes, a subpopulation of apoptotic adipocytes or dying adipocytes expressing adipocyte markers including adiponectin, FABP4 or both;
isolating from the subpopulation of apoptotic adipocytes a population of adipocyte-specific extracellular vesicles (adipomes) derived from the population of total WAT-derived extracellular vesicles;
enriching intact large adipomes (L-adipomes) from large EVs and small adipomes (S-adipomes) from small EVs that express the adipocyte markers adiponectin and FABP4 by positive selection employing adiponectin and FABP4 antibodies;
extracting cargo of the L-adipomes and the S-adipomes and determining their lipid profile;
comparing mRNA expression levels of the L-adipomes and S-adipomes from the subject to mRNA expression levels of a healthy control including adiponectin receptors AdipoR1 and AdipoR2; and
determining a level of expression of one or more genes encoding a panel of proteins that function in lipolytic signaling, lipogenesis, mitochondrial signaling, inflammation or a combination thereof in target cells;
wherein early diagnosis of the pathology can lead to improved outcome for the subject.

2. The method according to claim 1, wherein a source of the adipocyte population is plasma or white adipose tissue (WAT) comprising an adipocyte population including the subpopulation of apoptotic adipocytes or dying adipocytes and a non-adipocyte population comprising adipocyte precursor cells.

3. The method according to claim 1 wherein the L-adipomes are derived from large EVs comprising apoptotic bodies and macrovesicles and the S-adipomes are derived from small EVs comprising microvesicles and exosomes.

4. The method according to claim 1, wherein the pathology affects lipid metabolism, mitochondrial oxidative phosphorylation, inflammation, or a combination thereof.

5. The method according to claim 1, wherein the L-adipomes and S-adipomes derived from the adipocytes express adiponectin, FABP4, annexin V and perilipin.

6. The method according to claim 1, wherein size of the L-adipomes ranges from 200-400 nm and size of the S-adipomes ranges from 30-80 nm.

7. The method according to claim 1, wherein the panel of genes includes:
a gene encoding a protein of lipolytic signaling comprising beta-2-adrenergic receptor (ADRB2) protein;
a gene encoding a protein of lipogenesis comprising one or more of lipogenic SREBP1α and SREBP1c proteins
a genes encoding a protein of mitochondrial signaling comprising one or more of NADHD, ND1, ND2, SDHC, CYTB, COX1A, COX5A, APT6, ANT1 or PGC-1α protein; and
a gene encoding a protein of inflammatory signaling comprising TNFα, IFNg, or both proteins.

8. The method according to claim 1, wherein the pathology comprises polarization of a macrophage population derived from the subject at risk compared to a healthy subject.

9. The method according to claim 1, wherein the pathology places at risk cardiomyocytes, cardiac fibroblasts or both.

10. The method according to claim 1, wherein the pathology in an untreated subject progresses to hypertrophied cardiomyopathy, dilated cardiomyopathy and heart failure.

11. The method according to claim 10, wherein the hypertrophied cardiomyopathy comprises cardiac remodeling and cardiomyocyte dysfunction.

12. The method according to claim 1, wherein a source of the pathology is an infection.

13. The method according to claim 12, wherein the infection is a parasitic infection.

14. The method according to claim 13, wherein the parasitic infection is an infection with Trypanosoma cruzi, a causative agent of Chagas disease.

15. A method for modulating adipogenic signaling in a target cell population comprising:
selectively purifying from a population of adipocytes a subpopulation of apototic adipocytes or dying adipocytes expressing adipocyte markers including adiponectin, FABP4 or both;
isolating from the subpopulation of apoptotic and dying adipocytes a population of adipocyte-specific extracellular vesicles (adipomes) derived from the population of total WAT-derived extracellular vesicles, wherein size of the adipomes ranges from 200 nm to 1100 nm;
enriching intact large adipomes (L-adipomes) from large EVs and small adipomes (S-adipomes) from small EVs that express the adipocyte markers adiponectin and FABP4 by positive selection employing adiponectin and FABP4 antibodies;
extracting cargo of the L-adipomes and the S-adipomes and determining their cargo profile, wherein the cargo of the adipomes includes adipogenic mRNA;
treating the population of target cells with the adipomes comprising the adipogenic mRNA cargo; and
modulating gene expression of adiponectin, and downstream genes regulated by adiponectin.

16. The method according to claim 15, wherein a source of the adipocyte population is plasma or white adipose tissue (WAT) comprising an adipocyte population including a subpopulation of apoptotic adipocytes or dying adipocytes and a non-adipocyte population comprising adipocyte precursor cells.

17. The method according to claim 15, wherein the L-adipomes are derived from large EVs comprising apoptotic bodies and macrovesicles and the S-adipomes are derived from small EVs comprising microvesicles and exosomes.

18. The method according to claim 15, wherein the target cell population comprises macrophages, fibroblasts or both.

19. The method according to claim 15, wherein the adipogenic genes comprise mRNA for adipoq, Fabp4, and Pparg.

20. The method according to claim 15, wherein the downstream genes regulated by adiponectin include Ppara and adiponectin receptor R2 (AdipoR2).
